(19) United States
(12) Patent Application Publication
(10) Pub. No.: US 2023/0212323 A1
(43) Pub. Date: Jul. 6, 2023

(54) COMPOSITIONS AND METHODS FOR EPIGENOME EDITING

(71) Applicant: The Regents of the University of California, Oakland, CA (US)

(72) Inventors: Luke Gilbert, San Francisco, CA (US); Jonathan Weissman, San Francisco, CA (US); James Nunez, San Francisco, CA (US); Greg Pommier, San Francisco, CA (US)

(21) Appl. No.: 17/999,762

(22) PCT Filed: Jun. 4, 2021
(86) PCT No.: PCT/US2021/035937

§ 371 Date: Nov. 23, 2022

Related U.S. Application Data
(60) Provisional application No. 63/118,832, filed on Nov. 27, 2020.
(60) Provisional application No. 63/035,431, filed on Jun. 5, 2020.

(51) Int. Cl. C07K 19/00; C12N 15/63; C12N 9/22

(52) U.S. Cl. CPC C07K 19/00; C12N 15/63; C12N 9/22; C07K 2319/09; C07K 2319/80; C07K 2319/85

(57) ABSTRACT

Provided herein are, inter alia, compositions and methods for modulating gene expression.

### CROSS-REFERENCE TO RELATED APPLICATIONS

This application claims priority to U.S. Application No. 63/118,832 filed Nov. 27, 2020, and U.S. Application No. 63/035,431 filed Jun. 5, 2020, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under grant DARPA-BAA-16-59 awarded by The Defense Advanced Research Projects Agency. The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file 048536-690001WO_SequenceListing_ST25.txt, created 2021, x bytes, machine format IBM-PC, MS Windows operating system, is hereby incorporated by reference.

### BACKGROUND

Although gene editing using CRISPR-based technologies is a promising approach for treatment of diseases, particularly genetically-defined diseases, CRISPR-based gene editing relies on DNA breaks or base editing, which can result in off-target modifications, cell toxicity, or unpredictable DNA repair outcomes. Further, most CRISPR-based technologies are restricted to genome-editing, and may generate irreversible, deleterious changes. In contrast, modifications made through epigenetic editing may be long-term and are reversible, thus providing a safer approach to modulating gene expression. Epigenetic editing also provides opportunities for transforming both the DNA epigenetic code and the histone code, allowing for editing using different modalities and within various cellular and genetic contexts. Provided herein, inter alia, are solutions to these and other problems in the art.

### BRIEF SUMMARY

In an aspect is provided a fusion protein comprising from N-terminus to C-terminus, a demethylation domain, an XTEN linker, and a nuclease-deficient RNA-guided DNA endonuclease enzyme or a nuclease-deficient endonuclease enzyme. In aspects, the fusion protein further comprises a transcriptional activator. In aspects, the transcriptional activator is VP64, p65, Rta, or a combination of two or more thereof. In aspects, the fusion protein further comprises a nuclear localization sequence. In embodiments, the fusion protein comprises the nuclease-deficient RNA-guided DNA endonuclease enzyme. In embodiments, the fusion protein comprises the nuclease-deficient DNA endonuclease enzyme.

In an aspect is provided a fusion protein comprising from N-terminus to C-terminus, an RNA-binding sequence, an XTEN linker, and a transcriptional activator. In aspects, the transcriptional activator is VP64, p65, Rta, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In an aspect is provided a fusion protein comprising from N-terminus to C-terminus, a demethylation domain, an XTEN linker, a nuclease-deficient RNA-guided DNA endonuclease enzyme or a nuclease-deficient endonuclease enzyme, and a transcriptional activator. In aspects, the transcriptional activator is VP64, p65, Rta, or a combination of two or more thereof. In aspects, the fusion protein further comprises a nuclear localization sequence. In embodiments, the fusion protein comprises the nuclease-deficient RNA-guided DNA endonuclease enzyme. In embodiments, the fusion protein comprises the nuclease-deficient DNA endonuclease enzyme.

In an aspect is provided a fusion protein comprising from N-terminus to C-terminus, a demethylation domain, an XTEN linker, a nuclease-deficient RNA-guided DNA endonuclease enzyme or a nuclease-deficient endonuclease enzyme, and a nuclear localization sequence. In aspects, the fusion protein further comprises a transcriptional activator. In aspects, the transcriptional activator is VP64, p65, Rta, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In an aspect is provided a method of activating a target nucleic acid sequence in a cell, the method comprising: (i) delivering a first polynucleotide encoding a fusion protein described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA. In aspects, the target nucleic acid sequence comprises a CpG island. In aspects, the target nucleic acid sequence comprises a non-CpG island. In embodiments, the fusion protein comprises the nuclease-deficient RNA-guided DNA endonuclease enzyme. In embodiments, the method does not comprise step (ii) when the fusion protein comprises the nuclease-deficient DNA endonuclease enzyme.

In an aspect is provided a method of activating a target nucleic acid sequence or reactivating a silenced target nucleic acid sequence in a cell, the method comprising delivering a polynucleotide encoding a fusion protein described herein including embodiments thereof to a cell containing the silenced target nucleic acid; thereby reactivating the silenced target nucleic acid sequence in the cell. In embodiments, the fusion protein comprises a demethylation domain, an XTEN linker, a nuclease-deficient RNA-guided DNA endonuclease enzyme, an sgRNA, and a transcriptional activator. In aspects, the target nucleic acid sequence comprises a CpG island. In aspects, the target nucleic acid sequence comprises a non-CpG island.

These and other embodiments and aspects of the disclosure are described in detail herein.

### BRIEF DESCRIPTION OF DRAWINGS

FIG. 1 is a bar plot of HEK293T cells reactivating CRISPRoff-silenced H2B, Snrpn-GFP, or CLTA 9 days after Cas9-mediated knockout of DNMT1. The error bars are SD from three independent experiments.

FIG. 2 provides time course measurements of CLTA reactivation after increasing doses of 5-aza-dC in HEK293T cells with CLTA silenced by CRISPRoff. Percent cells with CLTA reactivated are shown. This plot shows that cells can reactivate the expression of CLTA via DNA demethylation.

FIG. 3 provides median CLTA-GFP fluorescence of CLTA reactivation after increasing doses of 5-aza-dC in HEK293T cells with CLTA silenced by CRISPRoff.

FIG. 4 is a schematic of gene reactivation experiment. Cells encoding CRISPRoff-silenced CLTA-GFP were transfected with plasmids encoding dCas9-TET1 and sgRNAs.

FIG. 5 is a schematic of four TET1 fusions to dCas9 (v1-v4) that were tested for CRISPRon gene reactivation.

FIG. 6 is a graph showing time course of CLTA reactivation after transfection of the four TET fusions in shown in FIG. 5 with a pool of sgRNAs targeting CLTA. The CLTA gene has a CpG island.

FIG. 7 is a bar graph showing a comparison of CLTA reactivation using the four TET fusions in FIG. 5 co-transfected with one sgRNA sequence or a pool of three sgRNAs. Error bars represent the range of two technical replicates.

FIG. 8 is a representative FACS plot of CLTA reactivation measured at 28 days post-transfection of TETv4 and targeting sgRNAs.

FIG. 9A is a bisulfite-PCR analysis of the CLTA CGI after TET1 reactivation show high levels of cytosine demethylation (white circles) compared to CRISPRoff silenced CLTA (black circles). Each row represents one sequencing read. The percent methylation of the locus is represented in the horizontal bar graph.

FIG. 9B provides a schematic of the CLTA CGI (green) with sgRNA binding sites annotated (a, b, c). The lollipop plot shading represents the percent of each CpG dinucleotide with methylated cytosine, as measured by bisulfite-PCR. The promoter, splicing, and CGI annotations were obtained from UCSC Genome Browser.

FIG. 10 is a schematic of the TETv4 and transactivator ribonucleoprotein complex mediated by a sgRNA encoding two MS2 RNA aptamers. Transactivator domains include monopartite, bipartite, and tripartite architectures of the VP16 tetramer VP64, the RELA activation domain (p65), and the viral transcriptional activator Rta.

FIG. 11 is a schematic of vectors that express a CLTA-targeting sgRNA and MS2 coat protein (MCP) fusion to various transcriptional activators.

FIG. 12 is a Violin plot that represents median CLTA-GFP fluorescence 2 days post-transfection of sgRNAs targeting CLTA and either dCas9 or dCas9 and MCP-fused transactivators into cells with endogenously expressed CLTA-GFP.

FIG. 13 is a bar graph showing a comparison of fold change in the fraction of CLTA-GFP reactivated cells measured two days post-transfection of TETv4 and MCP-fused transactivators. The data are displayed as the fold change compared to TETv4 alone, calculated from the mean of two technical replicates.

FIG. 14 shows bar graphs illustrating TET1 in combination with transactivators reactivates gene expression. Gene and plasmid expression levels were measured at multiple time points post-transfection.

FIGS. 15A-15B are violin plots illustrating that transient expression of Rta, p65-Rta and VP64-p65 transactivators resulted in significantly increased single cell gene expression within reactivated cells. FIG. 15B provides a comparison of median fluorescence of single cells that have reactivated CLTA-GFP, measured 28 days post-transfection. The data are representative of two technical replicates. * p value <0.05, ** p value <0.0005, *** p value, le-15 relative to the GFP positive population in the TETv4 condition by the Wilcoxon rank-sum test.

FIG. 16 is a bar graph showing gene reactivation by a TET1 fusion protein in cells with previously silenced genes. DYNC2LI1 and LAMP2 do not have a canonical CpG island.

FIG. 17 provides a time course of HEK293T cells with CLTA-GFP reactivation after transfection of sgRNAs targeting CLTA and either TETv4 only, or TETv4 along with various MCP-fused transactivator domains into cells with CRISPRoff-silenced CLTA. Untreated cells are represented in white circles. The error bars are SD from three independent experiments.

FIG. 18 provides a time course of HEK293T cells with CLTA-GFP reactivation after transfection of sgRNAs targeting CLTA and either dCas9-VPR or dCas9 along with various MCP-fused transactivator domains, or untransfected cells. The transfections were performed in the absence of TETv4 to measure persistent gene activation in the absence of DNA demethylation. The error bars are SD from three independent experiments.

FIGS. 19A-19D show fusion proteins and their gene reactivation. FIG. 19D is a graphic showing fusion proteins described herein, including GCP21 (SEQ ID NO:102), JKNp146 (SEQ ID NO:99), and JKNp147 (SEQ ID NO:101). FIGS. 19B-19D show gene reactivation of the CLTA gene, the DYNC2LI1 gene, and the histone H2B gene (respectively) after transfection of the fusion proteins, measured 13 days post-transfection.

### DETAILED DESCRIPTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acids, e.g. polynucleotides, contemplated herein include, but are not limited to, any type of RNA, e.g., mRNA, siRNA, miRNA, sgRNA, and guide RNA and any type of DNA, genomic DNA, plasmid DNA, and minicircle DNA, and any fragments thereof. In aspects, the nucleic acid is messenger RNA. In aspects, the messenger RNA is messenger ribonucleoprotein (RNP). The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In aspects, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complementary” or “complementarity” refers to the ability of a nucleic acid to form hydrogen bond(s) with another nucleic acid sequence by either traditional Watson-Crick or other non-traditional types. For example, the sequence A-G-T is complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions (i.e., stringent hybridization conditions).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. One of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al., supra.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “transcriptional regulatory sequence” as provided herein refers to a segment of DNA that is capable of increasing or decreasing transcription (e.g., expression) of a specific gene within an organism. Non-limiting examples of transcriptional regulatory sequences include promoters, enhancers, and silencers.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOM5 database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

In embodiments, the polynucleotide (e.g., gRNA) is a single-stranded ribonucleic acid. In aspects, the polynucleotide (e.g., gRNA) is from about 10 to about 200 nucleic acid residues in length. In aspects, the polynucleotide (e.g., gRNA) is from about 50 to about 150 nucleic acid residues in length. In aspects, the polynucleotide (e.g., gRNA) is from about 80 to about 140 nucleic acid residues in length. In aspects, the polynucleotide (e.g., gRNA) is from about 90 to about 130 nucleic acid residues in length. In aspects, the polynucleotide (e.g., gRNA) is from about 100 to about 120 nucleic acid residues in length. In aspects, the length of the polynucleotide (e.g., gRNA) is about 113 nucleic acid residues in length.

In general, a guide sequence (i.e., a DNA-targeting sequence) is any polynucleotide sequence having sufficient complementarity with a target polynucleotide sequence to hybridize with the target sequence (e.g., a genomic or mitochondrial DNA target sequence) and direct sequence-specific binding of a complex (e.g., CRISPR complex) to the target sequence. In aspects, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In aspects, the degree of complementarity between a guide sequence and its corresponding target sequence, when optimally aligned using a suitable alignment algorithm, is at least about 80%, 85%, 90%, 95%, or 100%. In aspects, the degree of complementarity is at least 90%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In aspects, a guide sequence is about or more than about 10, 20, 30, 35, 40, 45, 50, 75, or more nucleotides in length. In aspects, a guide sequence is about 10 to about 150, about 15 to about 100 nucleotides in length. In aspects, a guide sequence is less than about 75, 50, 45, 40, 35, 30, 25, 20, 15, 12, or fewer nucleotides in length. In aspects, the guide sequence is about or more than about 20 nucleotides in length. The ability of a guide sequence to direct sequence-specific binding of a complex (e.g., CRISPR complex) to a target sequence may be assessed by any suitable assay. For example, the components of a CRISPR system sufficient to form a complex (e.g., CRISPR complex), including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay known in the art. Similarly, cleavage of a target polynucleotide sequence may be evaluated in a test tube by providing the target sequence, components of a complex (e.g., CRISPR complex), including the guide sequence to be tested and a control guide sequence different from the test guide sequence, and comparing binding or rate of cleavage at the target sequence between the test and control guide sequence reactions. Other assays are possible, and will occur to those skilled in the art.

The terms “sgRNA,” “single guide RNA,” and “single guide RNA sequence” are used interchangeably and refer to the polynucleotide sequence including the crRNA sequence and optionally the tracrRNA sequence. The crRNA sequence includes a guide sequence (i.e., “guide” or “spacer”) and a tracr mate sequence (i.e., direct repeat(s)”). The term “guide sequence” refers to the sequence that specifies the target site. In aspects, the two RNA can be encoded separately by a crRNA and tracrRNA as 2 RNA molecules which then form an RNA/RNA complex due to complementary base pairing between the crRNA and tracrRNA (i.e., before being competent to bind to nuclease-deficient RNA-guided DNA endonuclease enzyme). In aspects, a first nucleic acid includes a tracrRNA sequence, and a separate second nucleic acid includes a gRNA sequence lacking a tracrRNA sequence. In aspects, the first nucleic acid including the tracrRNA sequence and the second nucleic acid including the gRNA sequence interact with one another, and optionally are included in a complex (e.g., CRISPR complex). Exemplary sgRNA, and their targeted sequences, are shown in Tables 2, 3, and 4.

TABLE 2

| Name | Targeted sequence (5′ to 3′) | sgRNA sequence (5′ to 3′) |
|---|---|---|
| A (JKNg156) | ACTGCGGAAATTTGAGCGT | ACGCUCAAAUUUCCGCAGU |
| | (SEQ ID NO: 37) | (SEQ ID NO: 38) |
| B (JKNg158) | AGGCAATGGCTGCACATGC | GCAUGUGCAGCCAUUGCCU |
| | (SEQ ID NO: 39) | (SEQ ID NO: 40) |
| C (JKNg160) | GACGCTTGGTTCTGAGGAG | CUCCUCAGAACCAAGCGUC |
| | (SEQ ID NO: 41) | (SEQ ID NO: 42) |

TABLE 3

| Name | Targeted sequence (5′ to 3′) | sgRNA sequence (5′ to 3′) |
|---|---|---|
| CD29, sgRNA-A | TCCGGAAACGCATTCCTCT | AGAGGAAUGCGUUUCCGGA |
| | (SEQ ID NO: 43) | (SEQ ID NO: 44) |
| CD29, sgRNA-B | CCGCGTCAGCCCGGCCCGG | CCGGGCCGGGCUGACGCGG |
| | (SEQ ID NO: 45) | (SEQ ID NO: 46) |
| CD29, sgRNA-C | CGACTCCCGCTGGGCCTCT | AGAGGCCCAGCGGGAGUCG |
| | (SEQ ID NO: 47) | (SEQ ID NO: 48) |
| CD81, sgRNA-A | ccgttgcgcgctcgctctc | gagagcgagcgcgcaacgg |
| | (SEQ ID NO: 49) | (SEQ ID NO: 50) |
| CD81, sgRNA-B | CCGCGCATCCTGCCAGGCC | GGCCUGGCAGGAUGCGCGG |
| | (SEQ ID NO: 51) | (SEQ ID NO: 52) |
| CD81, sgRNA-C | CCAACTTGGCGCGTTTCGG | CCGAAACGCGCCAAGUUGG |
| | (SEQ ID NO: 53) | (SEQ ID NO: 54) |
| CD151, sgRNA-A | ACCACGCGTCCGAGTCCGG | CCGGACUCGGACGCGUGGU |
| | (SEQ ID NO: 55) | (SEQ ID NO: 56) |
| CD151, sgRNA-B | TGCTCATTGTCCCTGGACA | UGUCCAGGGACAAUGAGCA |
| | (SEQ ID NO: 57) | (SEQ ID NO: 58) |
| CD151, sgRNA-C | GGACACCCTGCTCATTGTC | GACAAUGAGCAGGGUGUCC |
| | (SEQ ID NO: 59) | (SEQ ID NO: 60) |

TABLE 4

| Name | Targeted sequence (5′ to 3′) | sgRNA sequence (5′ to 3′) |
|---|---|---|
| Pcsk9 sgRNA-1 | TCCGGAAACGCATTCCTCT | AGAGGAAUGCGUUUCCGGA |
| | (SEQ ID NO: 43) | (SEQ ID NO: 44) |
| Pcsk9 sgRNA-2 | ACCGGCAGCCTGCGCGTCC | GGACGCGCAGGCUGCCGGU |
| | (SEQ ID NO: 61) | (SEQ ID NO: 62) |
| Pcsk9 sgRNA-3 | CGATGGGCACCCACTGCTC | GAGCAGUGGGUGCCCAUCG |
| | (SEQ ID NO: 63) | (SEQ ID NO: 64) |
| Pcsk9 sgRNA-4 | CCTTCACGTGGACGCGCAG | CUGCGCGUCCACGUGAAGG |
| | (SEQ ID NO: 65) | (SEQ ID NO: 66) |
| Pcsk9 sgRNA-5 | CGTGAAGGTGGAAGCCTTC | GAAGGCUUCCACCUUCACG |
| | (SEQ ID NO: 67) | (SEQ ID NO: 68) |
| Npc1 sgRNA-1 | CTCCTTGGTCAGGCGCCGG | CCGGCGCCUGACCAAGGAG |
| | (SEQ ID NO: 69) | (SEQ ID NO: 70) |
| Npc1 sgRNA-2 | TGGTCAGGCGCCGGTTCCG | CGGAACCGGCGCCUGACCA |
| | (SEQ ID NO: 71) | (SEQ ID NO: 72) |
| Npc1 sgRNA-3 | TAGAGGTCGCCTTCTCCTC | GAGGAGAAGGCGACCUCUA |
| | (SEQ ID NO: 73) | (SEQ ID NO: 74) |
| Npc1 sgRNA-4 | CGACGCTCGGGTCGCGGTG | CACCGCGACCCGAGCGUCG |
| | (SEQ ID NO: 75) | (SEQ ID NO: 76) |
| Npc1 sgRNA-5 | ATGCTGTCGCCGCGCGGGG | CCCCGCGCGGCGACAGCAU |
| | (SEQ ID NO: 77) | (SEQ ID NO: 78) |
| Spcs1 sgRNA-1 | CTCACCCTCACCGGAGCCA | UGGCUCCGGUGAGGGUGAG |
| | (SEQ ID NO: 79) | (SEQ ID NO: 80) |
| Spcs1 sgRNA-2 | CCGCAAACTTTACTCCTTA | UAAGGAGUAAAGUUUGCGG |
| | (SEQ ID NO: 81) | (SEQ ID NO: 82) |
| Spcs1 sgRNA-3 | CTCGGAGACATCCGCTTCC | GGAAGCGGAUGUCUCCGAG |
| | (SEQ ID NO: 60) | (SEQ ID NO: 60) |
| Spcs1 sgRNA-4 | CTCCTAAGATTGGCTTCAC | GUGAAGCCAAUCUUAGGAG |
| | (SEQ ID NO: 83) | (SEQ ID NO: 84) |
| Spcs1 sgRNA-5 | CCGGAGCCACTCCTAAGAT | AUCUUAGGAGUGGCUCCGG |
| | (SEQ ID NO: 85) | (SEQ ID NO: 86) |
| Cd81 sgRNA-1 | TTCTCTACCCTACGTCTCA | UGAGACGUAGGGUAGAGAA |
| | (SEQ ID NO: 87) | (SEQ ID NO: 88) |
| Cd81 sgRNA-2 | TACGTCTCATTCTCCGCAA | UUGCGGAGAAUGAGACGUA |
| | (SEQ ID NO: 89) | (SEQ ID NO: 90) |
| Cd81 sgRNA-3 | GCTAGGCCTCCAGCCCTTC | GAAGGGCUGGAGGCCUAGC |
| | (SEQ ID NO: 91) | (SEQ ID NO: 92) |
| Cd81 sgRNA-4 | ACAGGTGGCGCCGCAACTT | AAGUUGCGGCGCCACCUGU |
| | (SEQ ID NO: 93) | (SEQ ID NO: 94) |
| Cd81 sgRNA-5 | AGCCGGAGGCGCGAGAGTC | GACUCUCGCGCCUCCGGCU |
| | (SEQ ID NO: 95) | (SEQ ID NO: 96) |

The sequences in Tables 2, 3, and 4 are the targeting crRNA sequences. As an example, the full single guide RNA (sgRNA) for SEQ ID NO:38 is: GACGCUCAAAUUUCCGCAGUGUUUAAGAGCUAAGCUGGAAACAGCAUAGCAAGUUUAAAUAAGGCUAGUCCGUUAUCAACUUGAAAAAGUGGCACCGAGUCGGUGCUU UUUUU (SEQ ID NO:114). A common tracr sequence of each single guide for Sp Cas9 is GUUUAAGAGCUAAGCUGGAAACAGCAUAGCAAGUUUAAAUAAGGCUAGUCCGUU AUCAACUUGAAAAAGUGGCACCGAGUCGGUGCUUUUUUU (SEQ ID NO:115). The skilled artisan will appreciate that the sgRNA sequences in Tables 2, 3, and 4 are 19 base pairs and do not reflect that each sgRNA starts with a G which is required if expressed from a pol-III promoter for initiation of transcription. Thus, for SEQ ID NO:38, the sequence would be GACGCUCAAAUUUCCGCAGU (SEQ ID NO:116) rather than ACGCUCAAAUUUCCGCAGU (SEQ ID NO:38). In embodiments, SEQ ID NOS:38, 40, 42, 44, 46, 48, 50, 52, 54, 56, 58, 60, 62, 64, 66, 68, 70, 72, 74, 76, 78, 80, 82, 84, 86, 88, 90, 92, 94, and 96 each contain a G as the first nucleotide.

In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) comprises the tracr mate sequence hybridized to the tracr sequence. In general, degree of complementarity is with reference to the optimal alignment of the tracr mate sequence and tracrRNA sequence, along the length of the shorter of the two sequences. Optimal alignment may be determined by any suitable alignment algorithm, and may further account for secondary structures, such as self-complementarity within either the tracrRNA sequence or tracr mate sequence. In aspects, the degree of complementarity between the tracrRNA sequence and tracr mate sequence along the length of the shorter of the two when optimally aligned is about or more than about 25%, 30%, 40%, 50%, 60%, 70%, 80%, 90%, 95%, 97.5%, 99%, or higher. In aspects, the degree of complementarity is about or at least about 80%, 90%, 95%, or 100%. In aspects, the tracrRNA sequence is about or more than about 5, 10, 15, 20, 30, 40, 50, or more nucleotides in length. In aspects, the tracrRNA sequence and tracr mate sequence are contained within a single transcript, such that hybridization between the two produces a transcript having a secondary structure, such as a hairpin.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in aspects, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein (e.g., TET1, dCas9), the named protein includes any of the protein's naturally occurring forms, or variants or homologs that maintain the protein activity (e.g., within at least 50%, 80%, 90%, 95%, 96%, 97%, 98%, 99% or 100% activity compared to the native protein). In aspects, variants or homologs have at least 50%, 55%, 60%, 65%, 70%, 75%, 80%, 85%, 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In aspects, the protein is the protein as identified by its NCBI sequence reference. In aspects, the protein is the protein as identified by its NCBI sequence reference or functional fragment or homolog thereof.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class H of the CRISPR-Cas system.

A “nuclear localization sequence” or “nuclear localization signal” or “NLS” is a peptide that directs proteins to the nucleus. In aspects, the NLS includes five basic, positively charged amino acids. The NLS may be located anywhere on the peptide chain. In aspects, the NLS is an NLS derived from SV40. In aspects, the NLS includes the sequence set forth by SEQ ID NO:4. In aspects, the NLS is the sequence set forth by SEQ ID NO:4. In aspects, NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:4. In aspects, NLS has an amino acid sequence that has at least 75% sequence identity to SEQ ID NO:4. In aspects, NLS has an amino acid sequence that has at least 80% sequence identity to SEQ ID NO:4. In aspects, NLS has an amino acid sequence that has at least 85% sequence identity to SEQ ID NO:4. In aspects, NLS has an amino acid sequence that has at least 90% sequence identity to SEQ ID NO:4. In aspects, NLS has an amino acid sequence that has at least 95% sequence identity to SEQ ID NO:4. In aspects, NLS has an amino acid sequence of SEQ ID NO:4.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include nanoparticle encapsulation of the nucleic acids that encode the fusion protein (e.g., lipid nanoparticles, gold nanoparticles, and the like), calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “peptide linker” as provided herein is a linker including a peptide moiety. In embodiments, the peptide linker is a divalent peptide, such as an amino acid sequence attached at the N-terminus and the C-terminus to the remainder of the compound (e.g., fusion protein provided herein. The peptide linker may be a peptide moiety (a divalent peptide moiety) capable of being cleaved (e.g., a P2A cleavable polypeptide). A peptide linker as provided herein may also be referred to interchangeably as an amino acid linker. In aspects, the peptide linker includes 1 to about 80 amino acid residues. In aspects, the peptide linker includes 1 to about 70 amino acid residues. In aspects, the peptide linker includes 1 to about 60 amino acid residues. In aspects, the peptide linker includes 1 to about 50 amino acid residues. In aspects, the peptide linker includes 1 to about 40 amino acid residues. In aspects, the peptide linker includes 1 to about 30 amino acid residues. In aspects, the peptide linker includes 1 to about 25 amino acid residues. In aspects, the peptide linker includes 1 to about 20 amino acid residues. In aspects, the peptide linker includes about 2 to about 20 amino acid residues. In aspects, the peptide linker includes about 2 to about 19 amino acid residues. In aspects, the peptide linker includes about 2 to about 18 amino acid residues. In aspects, the peptide linker includes about 2 to about 17 amino acid residues. In aspects, the peptide linker includes about 2 to about 16 amino acid residues. In aspects, the peptide linker includes about 2 to about 15 amino acid residues. In aspects, the peptide linker includes about 2 to about 14 amino acid residues. In aspects, the peptide linker includes about 2 to about 13 amino acid residues. In aspects, the peptide linker includes about 2 to about 12 amino acid residues. In aspects, the peptide linker includes about 2 to about 11 amino acid residues. In aspects, the peptide linker includes about 2 to about 10 amino acid residues. In aspects, the peptide linker includes about 2 to about 9 amino acid residues. In aspects, the peptide linker includes about 2 to about 8 amino acid residues. In aspects, the peptide linker includes about 2 to about 7 amino acid residues. In aspects, the peptide linker includes about 2 to about 6 amino acid residues. In aspects, the peptide linker includes about 2 to about 5 amino acid residues. In aspects, the peptide linker includes about 2 to about 4 amino acid residues. In aspects, the peptide linker includes about 2 to about 3 amino acid residues. In aspects, the peptide linker includes about 3 to about 19 amino acid residues. In aspects, the peptide linker includes about 3 to about 18 amino acid residues. In aspects, the peptide linker includes about 3 to about 17 amino acid residues. In aspects, the peptide linker includes about 3 to about 16 amino acid residues. In aspects, the peptide linker includes about 3 to about 15 amino acid residues. In aspects, the peptide linker includes about 3 to about 14 amino acid residues. In aspects, the peptide linker includes about 3 to about 13 amino acid residues. In aspects, the peptide linker includes about 3 to about 12 amino acid residues. In aspects, the peptide linker includes about 3 to about 11 amino acid residues. In aspects, the peptide linker includes about 3 to about 10 amino acid residues. In aspects, the peptide linker includes about 3 to about 9 amino acid residues. In aspects, the peptide linker includes about 3 to about 8 amino acid residues. In aspects, the peptide linker includes about 3 to about 7 amino acid residues. In aspects, the peptide linker includes about 3 to about 6 amino acid residues. In aspects, the peptide linker includes about 3 to about 5 amino acid residues. In aspects, the peptide linker includes about 3 to about 4 amino acid residues. In aspects, the peptide linker includes about 10 to about 20 amino acid residues. In aspects, the peptide linker includes about 15 to about 20 amino acid residues. In aspects, the peptide linker includes about 2 amino acid residues. In aspects, the peptide linker includes about 3 amino acid residues. In aspects, the peptide linker includes about 4 amino acid residues. In aspects, the peptide linker includes about 5 amino acid residues. In aspects, the peptide linker includes about 6 amino acid residues. In aspects, the peptide linker includes about 7 amino acid residues. In aspects, the peptide linker includes about 8 amino acid residues. In aspects, the peptide linker includes about 9 amino acid residues. In aspects, the peptide linker includes about 10 amino acid residues. In aspects, the peptide linker includes about 11 amino acid residues. In aspects, the peptide linker includes about 12 amino acid residues. In aspects, the peptide linker includes about 13 amino acid residues. In aspects, the peptide linker includes about 14 amino acid residues. In aspects, the peptide linker includes about 15 amino acid residues. In aspects, the peptide linker includes about 16 amino acid residues. In aspects, the peptide linker includes about 17 amino acid residues. In aspects, the peptide linker includes about 18 amino acid residues. In aspects, the peptide linker includes about 19 amino acid residues. In aspects, the peptide linker includes about 20 amino acid residues. In aspects, the peptide linker includes about 21 amino acid residues. In aspects, the peptide linker includes about 22 amino acid residues. In aspects, the peptide linker includes about 23 amino acid residues. In aspects, the peptide linker includes about 24 amino acid residues. In aspects, the peptide linker includes about 25 amino acid residues.

The terms “XTEN,” “XTEN linker,” or “XTEN polypeptide” as used herein refer to an recombinant polypeptide (e.g. unstructured recombinant peptide) lacking hydrophobic amino acid residues. The development and use of XTEN can be found in, for example, Schellenberger et al., Nature Biotechnology 27, 1186-1190 (2009). In aspects, the XTEN linker includes the sequence set forth by SEQ ID NO:5, 6, or 98.

“Epitope tag” refers to a biological moiety, such as a peptide, that is genetically engineered into a recombinant protein and that functions as a universal epitope that is easily detected by commercially available assays or antibodies and that generally does not compromise the native structure or function of the protein.

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷AS, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g., iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

A detectable moiety is a monovalent detectable agent or a detectable agent capable of forming a bond with another composition. In aspects, the detectable agent is an epitope tag. In aspects, the epitope tag is an HA tag. In aspects, the HA tag includes the sequence set forth by SEQ ID NO:7. In aspects, the HA tag is the sequence set forth by SEQ ID NO:7. In aspects, the HA tag has an amino acid sequence that has at least 80% sequence identity to SEQ ID NO:7. In aspects, the HA tag has an amino acid sequence that has at least 85% sequence identity to SEQ ID NO:7. In aspects, the HA tag has an amino acid sequence that has at least 90% sequence identity to SEQ ID NO:7. In aspects, the HA tag has an amino acid sequence that has at least 95% sequence identity to SEQ ID NO:7.

In aspects, the detectable agent is a fluorescent protein. In aspects, the fluorescent protein is blue fluorescent protein (BFP). In aspects, the BFP includes the sequence set forth by SEQ ID NO:8. In aspects, the BFP is the sequence set forth by SEQ ID NO:8. In aspects, the BFP has an amino acid sequence that has at least 80% sequence identity to SEQ ID NO:8. In aspects, the BFP has an amino acid sequence that has at least 85% sequence identity to SEQ ID NO:8. In aspects, the BFP has an amino acid sequence that has at least 90% sequence identity to SEQ ID NO:8. In aspects, the BFP has an amino acid sequence that has at least 95% sequence identity to SEQ ID NO:8.

Radioactive substances (e.g., radioisotopes) that may be used as imaging and/or labeling agents in accordance with the aspects of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸ Ga, ⁷⁷AS, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Fr, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the aspects of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

“Contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a fusion protein as provided herein and a nucleic acid sequence (e.g., target DNA sequence).

As defined herein, the term “activation”, “activate”, “activating,” “enhance,” “reactivation,” “reactivate,” “reactivating” and the like when used in reference to a composition as provided herein (e.g., fusion protein, complex, nucleic acid, vector) refer to positively affecting (e.g., increasing) the activity (e.g., transcription) of a nucleic acid sequence (e.g., increasing transcription of a gene) relative to the activity of the nucleic acid sequence (e.g., transcription of a gene) in the absence of the composition (e.g., fusion protein, complex, nucleic acid, vector). Thus, activation or reactivation includes, at least in part increasing or upregulating (e.g., transcription) the expression, or preventing or reversing the decrease or delay of the expression (e.g., transcription) of the nucleic acid sequence. The activated or reactivated activity (e.g., transcription) may be 90%, 80%, 70%, 60%, 50%, 40%, 30%, 20%, 10%, or more than that in a control. In aspects, the activation or reactivation is 1.5-fold, 2-fold, 3-fold, 4-fold, 5-fold, 10-fold, or more in comparison to a control. In embodiments, activation may be activation of a gene that was previously silenced. In embodiments, reactivation may be reactivation of a gene that was previously silenced.

The term “enhancer” or “activator” as used herein refers to a region of DNA that may be bound by proteins (e.g., transcriptional activators) and/or polynucleotides to increase the likelihood that transcription of a gene will occur. Enhancers may be about 50 to about 35,000 base pairs in length. In embodiments, enhancers may be about 50 to about 1500 base pairs in length. Enhancers may be located downstream or upstream of the transcription initiation site that it regulates and may be hundreds to at least a million base pairs away from the transcription initiation site. In embodiments, the enhancer may be several hundreds of base pairs away from the transcription initiation site. In embodiments, the enhancer may be bound by at least one transcriptional activator (e.g., VP64, p65, Rta). In embodiments, the enhancer may be a target polynucleotide sequence suitable for epigenome editing. In embodiments, the enhancer may be targeted by one or more proteins and/or polynucleotides which may activate or reactivate transcription of a gene.

As defined herein, the term “inhibition”, “inhibit”, “inhibiting,” “repression,” repressing,” “silencing,” “silence” and the like when used in reference to a composition as provided herein (e.g., fusion protein, complex, nucleic acid, vector) refer to negatively affecting (e.g., decreasing) the activity (e.g., transcription) of a nucleic acid sequence (e.g., decreasing transcription of a gene) relative to the activity of the nuclei acid sequence (e.g., transcription of a gene) in the absence of the composition (e.g., fusion protein, complex, nucleic acid, vector). In aspects, inhibition refers to reduction of a disease or symptoms of disease (e.g., cancer). Thus, inhibition includes, at least in part, partially or totally blocking activation (e.g., transcription), or decreasing, preventing, or delaying activation (e.g., transcription) of the nucleic acid sequence. The inhibited activity (e.g., transcription) may be 90%, 80%, 70%, 60%, 50%, 40%, 30%, 20%, 10%, or less than that in a control. In aspects, the inhibition is 1.5-fold, 2-fold, 3-fold, 4-fold, 5-fold, 10-fold, or more in comparison to a control.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “demethylation domain” refers to a part of a protein sequence or structure that is capable of DNA demethylation. For example, a demethylation domain may remove a methyl group from a nucleobase (i.e. conversion of 5-methylcytosine to cytosine). In embodiments, demethylation domains include Ten-eleven translocation (TET) enzymes or functional domains of TET enzymes. In embodiments, the demethylation domain is a bacterial DNA demethylase.

The term “Ten-eleven translocation” or “TET” refers to the family of enzymes including TET1, TET2 and TET3. Without intending to be bound by any theory, TET enzymes may remove repressive 5mC marks and/or catalyze the oxidization of the methyl group of 5-methylcytosine (5mC) to yield 5-hydroxymethylcytosine (5hmC) and other oxidized methylcytosines, facilitating demethylation.

The term “TET1” or “TET1 protein” as provided herein includes any of the recombinant or naturally-occurring forms of Ten-eleven translocation methylcytosine dioxygenase 1 (TET1), also known as Methylcytosine dioxygenase TET1, COX-type zinc finger protein 6, Leukemia-associated protein with a CXXC domain, or variants or homologs thereof that maintain TET1 protein activity (e.g. within at least 50%, 80%, 90%, 95%, 96%, 97%, 98%, 99% or 100% activity compared to TET1 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TET1 protein polypeptide. In embodiments, TET1 protein is the protein as identified by the UniProt reference number Q8NFU7, or a variant, homolog or functional fragment thereof. In aspects, TET1 includes the amino acid sequence of SEQ ID NO:1. In aspects, TET1 has the amino acid sequence of SEQ ID NO:1. In aspects, TET1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:1. In aspects, TET1 has an amino acid sequence that has at least 75% sequence identity to SEQ ID NO:1. In aspects, TET1 has an amino acid sequence that has at least 80% sequence identity to SEQ ID NO:1. In aspects, TET1 has an amino acid sequence that has at least 85% sequence identity to SEQ ID NO:1. In aspects, TET1 has an amino acid sequence that has at least 90% sequence identity to SEQ ID NO:1. In aspects, TET1 has an amino acid sequence that has at least 95% sequence identity to SEQ ID NO:1. In aspects, TET1 includes the amino acid sequence of SEQ ID NO:86. In aspects, TET1 has the amino acid sequence of SEQ ID NO:86. In aspects, TET1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:86. In aspects, TET1 has an amino acid sequence that has at least 75% sequence identity to SEQ ID NO:86. In aspects, TET1 has an amino acid sequence that has at least 80% sequence identity to SEQ ID NO:86. In aspects, TET1 has an amino acid sequence that has at least 85% sequence identity to SEQ ID NO:86. In aspects, TET1 has an amino acid sequence that has at least 90% sequence identity to SEQ ID NO:86. In aspects, TET1 has an amino acid sequence that has at least 95% sequence identity to SEQ ID NO:86. In aspects, TET1 includes the amino acid sequence of SEQ ID NO:97. In aspects, TET1 has the amino acid sequence of SEQ ID NO:97. In aspects, TET1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:97. In aspects, TET1 has an amino acid sequence that has at least 75% sequence identity to SEQ ID NO:97. In aspects, TET1 has an amino acid sequence that has at least 80% sequence identity to SEQ ID NO:97. In aspects, TET1 has an amino acid sequence that has at least 85% sequence identity to SEQ ID NO:97. In aspects, TET1 has an amino acid sequence that has at least 90% sequence identity to SEQ ID NO:97. In aspects, TET1 has an amino acid sequence that has at least 95% sequence identity to SEQ ID NO:97.

The term “TET2” or “TET2 protein” as provided herein includes any of the recombinant or naturally-occurring forms of Ten-eleven translocation methylcytosine dioxygenase 2 (TET2), also known as Methylcytosine dioxygenase TET2, or variants or homologs thereof that maintain TET2 protein activity (e.g. within at least 50%, 80%, 90%, 95%, 96%, 97%, 98%, 99% or 100% activity compared to TET2 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TET2 protein polypeptide. In embodiments, TET2 protein is the protein as identified by the UniProt reference number Q6N021, or a variant, homolog or functional fragment thereof. In aspects, TET2 includes the amino acid sequence of SEQ ID NO:2. In aspects, TET2 has the amino acid sequence of SEQ ID NO:2. In aspects, TET2 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:2. In aspects, TET2 has an amino acid sequence that has at least 75% sequence identity to SEQ ID NO:2. In aspects, TET2 has an amino acid sequence that has at least 80% sequence identity to SEQ ID NO:2. In aspects, TET2 has an amino acid sequence that has at least 85% sequence identity to SEQ ID NO:2. In aspects, TET2 has an amino acid sequence that has at least 90% sequence identity to SEQ ID NO:2. In aspects, TET2 has an amino acid sequence that has at least 95% sequence identity to SEQ ID NO:2.

The term “TET3” or “TET3 protein” as provided herein includes any of the recombinant or naturally-occurring forms of Ten-eleven translocation methylcytosine dioxygenase 3 (TET3), also known as Methylcytosine dioxygenase TET3, or variants or homologs thereof that maintain TET3 protein activity (e.g. within at least 50%, 80%, 90%, 95%, 96%, 97%, 98%, 99% or 100% activity compared to TET3 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring TET3 protein polypeptide. In embodiments, TET3 protein is the protein as identified by the UniProt reference number 043151, or a variant, homolog or functional fragment thereof. In aspects, TET3 includes the amino acid sequence of SEQ ID NO:3. In aspects, TET3 has the amino acid sequence of SEQ ID NO:3. In aspects, TET3 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:3. In aspects, TET3 has an amino acid sequence that has at least 75% sequence identity to SEQ ID NO:3. In aspects, TET3 has an amino acid sequence that has at least 80% sequence identity to SEQ ID NO:3. In aspects, TET3 has an amino acid sequence that has at least 85% sequence identity to SEQ ID NO:3. In aspects, TET3 has an amino acid sequence that has at least 90% sequence identity to SEQ ID NO:3. In aspects, TET3 has an amino acid sequence that has at least 95% sequence identity to SEQ ID NO:3.

The term “transcriptional activator,” “activator” and the like refer, in the usual and customary sense, to a protein (i.e. a transcription factor) that increases gene transcription of a gene or set of genes. For example, transcriptional activators may be DNA-binding proteins that bind to enhancers or promoter-proximal elements. In embodiments, the transcriptional activator is VP64, p65, or Rta. In embodiments, the transcriptional activator may increase gene transcription of a gene or a set of genes that was/were previously silenced. Transcriptional activators and uses thereof may be found, for example, in Tanenbaum et al., A Protein-Tagging System for Signal Amplification in Gene Expression and Fluorescence Imaging. Cell. 2014 Oct. 23; 159(3):635-46 and Zalatan et al., Engineering Complex Synthetic Transcriptional Programs With CRISPR RNA Scaffolds. Cell. 2015 Jan. 15; 160(1-2)339-50, which are incorporated herein by reference in their entirety and for all purposes.

The term “p65” or “p65 protein” as provided herein includes any of the recombinant or naturally-occurring forms of Transcription factor p65 (p65), also known as Nuclear factor NF-kappa-B p65 subunit, or variants or homologs thereof that maintain p65 protein activity (e.g. within at least 50%, 80%, 90%, 95%, 96%, 97%, 98%, 99% or 100% activity compared to p65 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring p65 protein polypeptide. In embodiments, p65 protein is the protein as identified by the UniProt reference number Q04206, or a variant, homolog or functional fragment thereof. In aspects, p65 includes the amino acid sequence of SEQ ID NO:13. In aspects, p65 has the amino acid sequence of SEQ ID NO:13. In aspects, p65 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:13. In aspects, p65 has an amino acid sequence that has at least 75% sequence identity to SEQ ID NO:13. In aspects, p65 has an amino acid sequence that has at least 80% sequence identity to SEQ ID NO:13. In aspects, p65 has an amino acid sequence that has at least 85% sequence identity to SEQ ID NO:13. In aspects, p65 has an amino acid sequence that has at least 90% sequence identity to SEQ ID NO:13. In aspects, p65 has an amino acid sequence that has at least 95% sequence identity to SEQ ID NO:13. In aspects, p65 includes the amino acid sequence of SEQ ID NO:14. In aspects, p65 has the amino acid sequence of SEQ ID NO:14. In aspects, p65 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:14. In aspects, p65 has an amino acid sequence that has at least 75% sequence identity to SEQ ID NO:14. In aspects, p65 has an amino acid sequence that has at least 80% sequence identity to SEQ ID NO:14. In aspects, p65 has an amino acid sequence that has at least 85% sequence identity to SEQ ID NO:14. In aspects, p65 has an amino acid sequence that has at least 90% sequence identity to SEQ ID NO:14. In aspects, p65 has an amino acid sequence that has at least 95% sequence identity to SEQ ID NO:14. In aspects, p65 includes the amino acid sequence of SEQ ID NO:100. In aspects, p65 has the amino acid sequence of SEQ ID NO:100. In aspects, p65 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:100. In aspects, p65 has an amino acid sequence that has at least 75% sequence identity to SEQ ID NO:100. In aspects, p65 has an amino acid sequence that has at least 80% sequence identity to SEQ ID NO:100. In aspects, p65 has an amino acid sequence that has at least 85% sequence identity to SEQ ID NO:100. In aspects, p65 has an amino acid sequence that has at least 90% sequence identity to SEQ ID NO:100. In aspects, p65 has an amino acid sequence that has at least 95% sequence identity to SEQ ID NO:100.

The term “Rta” or “Rta protein” as provided herein includes any of the recombinant or naturally-occurring forms of Replication and transcription activator (Rta), also known as R transactivator, Immediate-early protein Rta, or variants or homologs thereof that maintain Rta protein activity (e.g. within at least 50%, 80%, 90%, 95%, 96%, 97%, 98%, 99% or 100% activity compared to Rta protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Rta protein polypeptide. In embodiments, Rta protein is the protein as identified by the UniProt reference number P03209, or a variant, homolog or functional fragment thereof. In aspects, Rta includes the amino acid sequence of SEQ ID NO:15. In aspects, Rta has the amino acid sequence of SEQ ID NO:15. In aspects, Rta has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:15. In aspects, Rta has an amino acid sequence that has at least 75% sequence identity to SEQ ID NO:15. In aspects, Rta has an amino acid sequence that has at least 80% sequence identity to SEQ ID NO:15. In aspects, Rta has an amino acid sequence that has at least 85% sequence identity to SEQ ID NO:15. In aspects, Rta has an amino acid sequence that has at least 90% sequence identity to SEQ ID NO:15. In aspects, Rta has an amino acid sequence that has at least 95% sequence identity to SEQ ID NO:15. In aspects, Rta includes the amino acid sequence of SEQ ID NO:16. In aspects, Rta has the amino acid sequence of SEQ ID NO:16. In aspects, Rta has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:16. In aspects, Rta has an amino acid sequence that has at least 75% sequence identity to SEQ ID NO:16. In aspects, Rta has an amino acid sequence that has at least 80% sequence identity to SEQ ID NO:16. In aspects, Rta has an amino acid sequence that has at least 85% sequence identity to SEQ ID NO:16. In aspects, Rta has an amino acid sequence that has at least 90% sequence identity to SEQ ID NO:16. In aspects, Rta has an amino acid sequence that has at least 95% sequence identity to SEQ ID NO:16.

The term “VP64” or “VP64 protein” as provided herein includes any of the recombinant or naturally-occurring forms of Tegument protein VP16 (VP64), also known as Alpha trans-inducing protein, Alpha-TIF, or variants or homologs thereof that maintain VP64 protein activity (e.g. within at least 50%, 80%, 90%, 95%, 96%, 97%, 98%, 99% or 100% activity compared to VP64 protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring VP64 protein polypeptide. In embodiments, VP64 protein is the protein as identified by the UniProt reference number P06492, or a variant, homolog or functional fragment thereof. In aspects, VP64 includes the amino acid sequence of SEQ ID NO:17. In aspects, VP64 has the amino acid sequence of SEQ ID NO:17. In aspects, VP64 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:17. In aspects, VP64 has an amino acid sequence that has at least 75% sequence identity to SEQ ID NO:17. In aspects, VP64 has an amino acid sequence that has at least 80% sequence identity to SEQ ID NO:17. In aspects, VP64 has an amino acid sequence that has at least 85% sequence identity to SEQ ID NO:17. In aspects, VP64 has an amino acid sequence that has at least 90% sequence identity to SEQ ID NO:17. In aspects, VP64 has an amino acid sequence that has at least 95% sequence identity to SEQ ID NO:17. In aspects, VP64 includes the amino acid sequence of SEQ ID NO:18. In aspects, VP64 has the amino acid sequence of SEQ ID NO:18. In aspects, VP64 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:18. In aspects, VP64 has an amino acid sequence that has at least 75% sequence identity to SEQ ID NO:18. In aspects, VP64 has an amino acid sequence that has at least 80% sequence identity to SEQ ID NO:18. In aspects, VP64 has an amino acid sequence that has at least 85% sequence identity to SEQ ID NO:18. In aspects, VP64 has an amino acid sequence that has at least 90% sequence identity to SEQ ID NO:18. In aspects, VP64 has an amino acid sequence that has at least 95% sequence identity to SEQ ID NO:18.

The term “MCP” or “MCP protein” as provided herein includes any of the recombinant or naturally-occurring forms of Capsid protein (MCP), also known as CP, coat protein, or variants or homologs thereof that maintain MCP protein activity (e.g. within at least 50%, 80%, 90%, 95%, 96%, 97%, 98%, 99% or 100% activity compared to MCP protein). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MCP protein polypeptide. In embodiments, MCP protein is the protein as identified by the UniProt reference number P03612, or a variant, homolog or functional fragment thereof. In aspects, MCP includes the amino acid sequence of SEQ ID NO:21. In aspects, MCP has the amino acid sequence of SEQ ID NO:21. In aspects, MCP has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:21. In aspects, MCP has an amino acid sequence that has at least 75% sequence identity to SEQ ID NO:21. In aspects, MCP has an amino acid sequence that has at least 80% sequence identity to SEQ ID NO:21. In aspects, MCP has an amino acid sequence that has at least 85% sequence identity to SEQ ID NO:21. In aspects, MCP has an amino acid sequence that has at least 90% sequence identity to SEQ ID NO:21. In aspects, MCP has an amino acid sequence that has at least 95% sequence identity to SEQ ID NO:21

The term “nuclease-deficient RNA-guided DNA endonuclease enzyme” and the like refer, in the usual and customary sense, to an RNA-guided DNA endonuclease (e.g. a mutated form of a naturally occurring RNA-guided DNA endonuclease) that targets a specific phosphodiester bond within a DNA polynucleotide, wherein the recognition of the phosphodiester bond is facilitated by a separate polynucleotide sequence (for example, a RNA sequence (e.g., single guide RNA (sgRNA)), but is incapable of cleaving the target phosphodiester bond to a significant degree (e.g. there is no measurable cleavage of the phosphodiester bond under physiological conditions). A nuclease-deficient RNA-guided DNA endonuclease thus retains DNA-binding ability (e.g. specific binding to a target sequence) when complexed with a polynucleotide (e.g., sgRNA), but lacks significant endonuclease activity (e.g. any amount of detectable endonuclease activity). In aspects, the nuclease-deficient RNA-guided DNA endonuclease enzyme is a CRISPR-associated protein. In aspects, the nuclease-deficient RNA-guided DNA endonuclease enzyme is dCas9, dCas12a, dCpf1, ddCpf1, Cas-phi, a nuclease-deficient Cas9 variant, a nuclease-deficient Class II CRISPR endonuclease, a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain, a winged helix domain, a helix-turn-helix motif, a helix-loop-helix domain, an HMB-box domain, a Wor3 domain, an OB-fold domain, an immunoglobulin domain, or a B3 domain. In aspects, the nuclease-deficient RNA-guided DNA endonuclease enzyme is a leucine zipper domain. In aspects, the nuclease-deficient RNA-guided DNA endonuclease enzyme is a winged helix domain. In aspects, the nuclease-deficient RNA-guided DNA endonuclease enzyme is a helix-turn-helix motif. In aspects, the nuclease-deficient RNA-guided DNA endonuclease enzyme is a helix-loop-helix domain. In aspects, the nuclease-deficient RNA-guided DNA endonuclease enzyme is an HMB-box domain. In aspects, the nuclease-deficient RNA-guided DNA endonuclease enzyme is a Wor3 domain. In aspects, the nuclease-deficient RNA-guided DNA endonuclease enzyme is an OB-fold domain. In aspects, the nuclease-deficient RNA-guided DNA endonuclease enzyme is an immunoglobulin domain. In aspects, the nuclease-deficient RNA-guided DNA endonuclease enzyme is a B3 domain. In aspects, the nuclease-deficient RNA-guided DNA endonuclease enzyme is dCas9, dCas12a, ddCpf1, Cas-phi, a nuclease-deficient Cas9 variant, or a nuclease-deficient Class II CRISPR endonuclease. In aspects, the nuclease-deficient RNA-guided DNA endonuclease enzyme is dCas9. In aspects, the nuclease-deficient RNA-guided DNA endonuclease enzyme is dCas9 from S. pyogenes. In aspects, the nuclease-deficient RNA-guided DNA endonuclease enzyme is dCas9 from S. aureus. In aspects, the nuclease-deficient RNA-guided DNA endonuclease enzyme is dCas12a. In aspects, the nuclease-deficient RNA-guided DNA endonuclease enzyme is dCas12a from Lachnospiraceae bacterium. In aspects, the nuclease-deficient RNA-guided DNA endonuclease enzyme is dCas12. In aspects, the nuclease-deficient RNA-guided DNA endonuclease enzyme is ddCas12a. In aspects, the nuclease-deficient RNA-guided DNA endonuclease enzyme is Cas-phi.

The term “CRISPR-associated protein” or “CRISPR protein” refers to any CRISPR protein that functions as a nuclease-deficient RNA-guided DNA endonuclease enzyme, i.e., a CRISPR protein in which catalytic sites for endonuclease activity are defective or lack activity. Exemplary CRISPR proteins include dCas9, dCpf1, ddCpf1, dCas12, ddCas12, dCas12a Cas-phi, a nuclease-deficient Cas9 variant, a nuclease-deficient Class II CRISPR endonuclease, and the like.

The term “nuclease-deficient DNA endonuclease enzyme” refers to a DNA endonuclease (e.g. a mutated form of a naturally occurring DNA endonuclease) that targets a specific phosphodiester bond within a DNA polynucleotide, but that does not require an RNA guide. In embodiments, the “nuclease-deficient DNA endonuclease enzyme” is a zinc finger domain or a transcription activator-like effector (TALE).

In embodiments, the nuclease-deficient DNA endonuclease enzyme is a “zinc finger domain.” The term “zinc finger domain” or “zinc finger binding domain” or “zinc finger DNA binding domain” are used interchangeably and refer to a protein, or a domain within a larger protein, that binds DNA in a sequence-specific manner through one or more zinc fingers, which are regions of amino acid sequence within the binding domain whose structure is stabilized through coordination of a zinc ion. In embodiments, the zinc finger domain is non-naturally occurring in that it is engineered to bind to a target site of choice. In aspects, the zinc finger binding domain refers to a protein, a domain within a larger protein, or a nuclease-deficient RNA-guided DNA endonuclease enzyme that is capable of binding to any zinc finger known in the art, such as the C2H2 type, the CCHC type, the PHD type, or the RING type of zinc fingers.

As used herein, a “zinc finger” is a polypeptide structural motif folded around a bound zinc cation. In embodiments, the polypeptide of a zinc finger has a sequence of the form X3-Cys-X2-4-Cys-X¹²-His-X3_5-His-X4, wherein X is any amino acid (e.g., X2-4 indicates an oligopeptide 2-4 amino acids in length). There is generally a wide range of sequence variation in the 28-31 amino acids of the known zinc finger polypeptides. Only the two consensus histidine residues and two consensus cysteine residues bound to the central zinc atom are invariant. Of the remaining residues, three to five are highly conserved, while there may be significant variation among the other residues. Despite the wide range of sequence variation in the polypeptide, zinc fingers of this type have a similar three dimensional structure. However, there is a wide range of binding specificities among the different zinc fingers, i.e. different zinc fingers bind double stranded polynucleotides having a wide range of nucleotides sequences. In aspects, the zinc finger is the C2H2 type. In aspects, the zinc finger is the CCHC type. In aspects, the zinc finger is the PHD type. In aspects, the zinc finger is the RING type.

In embodiments, the nuclease-deficient DNA endonuclease enzyme is a TALE. “TALE” or “transcription activator-like effector” refer to artificial restriction enzymes generated by fusing the TAL effector DNA binding domain to a DNA cleavage domain. TALCS enable efficient, programmable, and specific DNA cleavage and represent powerful tools for genome editing in situ. Transcription activator-like effectors (TALEs) can be quickly engineered to bind practically any DNA sequence. The term TALE, as used herein, is broad and includes a monomeric TALE that can cleave double stranded. DNA without assistance from another TALE. The term TALE is also used to refer to one or both members of a pair of TALES that are engineered to work together to cleave DNA at the same site. TALES that work together may be referred to as a left-TALE and a right-TALE, which references the handedness of DNA. TALE are proteins secreted by Xanthomonas bacteria. The DNA binding domain contains a highly conserved 33-34 amino acid sequence with the exception of the 12th and 13th amino acids. These two locations are highly variable (repeat variable diresidue (MT N and show a strong correlation with specific nucleotide recognition. This simple relationship between amino acid sequence and DNA recognition has allowed for the engineering of specific DNA binding domains by selecting a combination of repeat segments containing the appropriate RVDs.

In embodiments, the nuclease-deficient RNA-guided DNA endonuclease enzyme is dCas9. The terms “dCas9” or “dCas9 protein” as referred to herein is a Cas9 protein in which both catalytic sites for endonuclease activity are defective or lack activity. In aspects, the dCas9 protein has mutations at positions corresponding to D10A and H840A of S. pyogenes Cas9. In aspects, the dCas9 protein lacks endonuclease activity due to point mutations at both endonuclease catalytic sites (RuvC and HNH) of wild type Cas9. The point mutations can be D10A and H840A. In aspects, the dCas9 has substantially no detectable endonuclease (e.g., endodeoxyribonuclease) activity. In aspects, dCas9 includes the amino acid sequence of SEQ ID NO:9. In aspects, dCas9 has the amino acid sequence of SEQ ID NO:9. In aspects, dCas9 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:9. In aspects, dCas9 has an amino acid sequence that has at least 75% sequence identity to SEQ ID NO:9. In aspects, dCas9 has an amino acid sequence that has at least 80% sequence identity to SEQ ID NO:9. In aspects, dCas9 has an amino acid sequence that has at least 85% sequence identity to SEQ ID NO:9. In aspects, dCas9 has an amino acid sequence that has at least 90% sequence identity to SEQ ID NO:9. In aspects, dCas9 has an amino acid sequence that has at least 95% sequence identity to SEQ ID NO:9.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

In embodiments, the nuclease-deficient RNA-guided DNA endonuclease enzyme is “ddCpf1” or “ddCas12a”. The terms “DNAse-dead Cpf1” or “ddCpf1” refer to mutated Acidaminococcus sp. Cpf1 (AsCpf1) resulting in the inactivation of Cpf1 DNAse activity. In aspects, ddCpf1 includes an E993A mutation in the RuvC domain of AsCpf1. In aspects, the ddCpf1 has substantially no detectable endonuclease (e.g., endodeoxyribonuclease) activity. In aspects, ddCpf1 includes the amino acid sequence of SEQ ID NO:10. In aspects, ddCpf1 has the amino acid sequence of SEQ ID NO:10. In aspects, ddCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:10. In aspects, ddCpf1 has an amino acid sequence that has at least 75% sequence identity to SEQ ID NO:10. In aspects, ddCpf1 has an amino acid sequence that has at least 80% sequence identity to SEQ ID NO:10. In aspects, ddCpf1 has an amino acid sequence that has at least 85% sequence identity to SEQ ID NO:10. In aspects, ddCpf1 has an amino acid sequence that has at least 90% sequence identity to SEQ ID NO:10. In aspects, ddCpf1 has an amino acid sequence that has at least 95% sequence identity to SEQ ID NO:10.

In embodiments, the nuclease-deficient RNA-guided DNA endonuclease enzyme is dLbCpf1. The term “dLbCpf1: refers to mutated Cpf1 from Lachnospiraceae bacterium ND2006 (LbCpf1) that lacks DNAse activity. In aspects, dLbCpf1 includes a D832A mutation. In aspects, the dLbCpf1 has substantially no detectable endonuclease (e.g., endodeoxyribo-nuclease) activity. In aspects, dLbCpf1 includes the amino acid sequence of SEQ ID NO:11. In aspects, dLbCpf1 has the amino acid sequence of SEQ ID NO:11. In aspects, dLbCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:11. In aspects, dLbCpf1 has an amino acid sequence that has at least 75% sequence identity to SEQ ID NO:11. In aspects, dLbCpf1 has an amino acid sequence that has at least 80% sequence identity to SEQ ID NO:11. In aspects, dLbCpf1 has an amino acid sequence that has at least 85% sequence identity to SEQ ID NO:11. In aspects, dLbCpf1 has an amino acid sequence that has at least 90% sequence identity to SEQ ID NO:11. In aspects, dLbCpf1 has an amino acid sequence that has at least 95% sequence identity to SEQ ID NO:11.

In embodiments, the nuclease-deficient RNA-guided DNA endonuclease enzyme is dFnCpf1. The term “dFnCpf1” refers to mutated Cpf1 from Francisella novicida U112 (FnCpf1) that lacks DNAse activity. In aspects, dFnCpf1 includes a D917A mutation. In aspects, the dFnCpf1 has substantially no detectable endonuclease (e.g., endodeoxyribo-nuclease) activity. In aspects, dFnCpf1 includes the amino acid sequence of SEQ ID NO: 12. In aspects, dFnCpf1 has the amino acid sequence of SEQ ID NO:12. In aspects, dFnCpf1 has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:12. In aspects, dFnCpf1 has an amino acid sequence that has at least 75% sequence identity to SEQ ID NO:12. In aspects, dFnCpf1 has an amino acid sequence that has at least 80% sequence identity to SEQ ID NO:12. In aspects, dFnCpf1 has an amino acid sequence that has at least 85% sequence identity to SEQ ID NO:12. In aspects, dFnCpf1 has an amino acid sequence that has at least 90% sequence identity to SEQ ID NO:12. In aspects, dFnCpf1 has an amino acid sequence that has at least 95% sequence identity to SEQ ID NO:12.

A “Cpf1” or “Cpf1 protein” as referred to herein includes any of the recombinant or naturally-occurring forms of the Cpf1 (CRISPR from Prevotella and Francisella 1) endonuclease or variants or homologs thereof that maintain Cpf1 endonuclease enzyme activity (e.g. within at least 50%, 80%, 90%, 95%, 96%, 97%, 98%, 99% or 100% activity compared to Cpf1). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cpf1 protein. In aspects, the Cpf1 protein is substantially identical to the protein identified by the UniProt reference number U2UMQ6 or a variant or homolog having substantial identity thereto. In aspects, the Cpf1 protein is identical to the protein identified by the UniProt reference number U2UMQ6. In aspects, the Cpf1 protein has at least 75% sequence identity to the amino acid sequence of the protein identified by the UniProt reference number U2UMQ6. In aspects, the Cpf1 protein has at least 80% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 85% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 90% sequence identity to the amino acid sequence of the protein identified by the UniProt reference number U2UMQ6. In aspects, the Cpf1 protein is identical to the protein identified by the UniProt reference number U2UMQ6. In aspects, the Cpf1 protein has at least 95% sequence identity to the amino acid sequence of the protein identified by the UniProt reference number U2UMQ6.

In embodiments, the nuclease-deficient RNA-guided DNA endonuclease enzyme is a nuclease-deficient Cas9 variant. The term “nuclease-deficient Cas9 variant” refers to a Cas9 protein having one or more mutations that increase its binding specificity to PAM compared to wild type Cas9 and further include mutations that render the protein incapable of or having severely impaired endonuclease activity. Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). The binding specificity of nuclease-deficient Cas9 variants to PAM can be determined by any method known in the art. Descriptions and uses of known Cas9 variants may be found, for example, in Shmakov et al., Diversity and evolution of class 2 CRISPR-Cas systems. Nat. Rev. Microbiol. 15, 2017 and Cebrian-Serrano et al, CRISPR-Cas orthologues and variants: optimizing the repertoire, specificity and delivery of genome engineering tools. Mamm. Genome 7-8, 2017, which are incorporated herein by reference in their entirety and for all purposes. Exemplary Cas9 variants are listed in the Table 1 below.

TABLE 1

| Cas9 Variants | PAM domains | References |
|---|---|---|
| Strep pyogenes (Sp) Cas9 | NGG | Hsu et al. 2014 Cell |
| Staph aureus | NNGRRT or NNGRR | Ran et al. |
| (Sa) Cas9 | NNGGGT, NNGAAT, | 2015 Nature |
| | NNGAGT (Zetsche) | |
| SpCas9 VQR mutant | NGAG > NGAT = | Kleinstiver |
| (D1135V, R1335Q, | NGAA > NGAC | et al. 2015 |
| T1337R) | NGCG | Nature |
| SpCas9 VRER mutant | NGCG | Kleinstiver |
| (D1135V/G1218R/ | | et al. 2015 |
| R1335E/T1337R) | | Nature |
| SpCas9 D1135E | NGG, greater fidelity, | Kleinstiver |
| | less cutting at | et al. 2015 |
| | NAG and NGA sites | Nature |
| eSpCas9 1.1 mutant | NGG | Slaymaker |
| (K848A/K1003A/ | | et al. Science |
| R1060A) | | 2015 |
| SpCas9 HF1 | NGG | Kleinstiver |
| (Q695A, Q926A, | | et al. 2016 |
| N497A, R661A) | | Nature |
| AsCpf1 | TTTN (5' of sgRNA) | Zetsche et al. 2015 Cell |
| HypaCas9 | | Chen et al., Nature |
| (N692A, M694A, | | volume 550, pages 407- |
| Q695A, H698A) | | 410 (19 Oct. 2017) |

In embodiments, the nuclease-deficient RNA-guided DNA endonuclease enzyme is a nuclease-deficient Class II CRISPR endonuclease. The term “nuclease-deficient Class II CRISPR endonuclease” as used herein refers to any Class II CRISPR endonuclease having mutations resulting in reduced, impaired, or inactive endonuclease activity.

In embodiments, the peptide linker is a XTEN linker. In aspects, the XTEN linker includes about 16 to about 864 amino acid residues. In aspects, the XTEN linker includes about 16 to about 80 amino acid residues. In aspects, the XTEN linker includes about 17 to about 80 amino acid residues. In aspects, the XTEN linker includes about 18 to about 80 amino acid residues. In aspects, the XTEN linker includes about 19 to about 80 amino acid residues. In aspects, the XTEN linker includes about 20 to about 80 amino acid residues. In aspects, the XTEN linker includes about 30 to about 80 amino acid residues. In aspects, the XTEN linker includes about 40 to about 80 amino acid residues. In aspects, the XTEN linker includes about 50 to about 80 amino acid residues. In aspects, the XTEN linker includes about 60 to about 80 amino acid residues. In aspects, the XTEN linker includes about 70 to about 80 amino acid residues. In aspects, the XTEN linker includes about 16 to about 70 amino acid residues. In aspects, the XTEN linker includes about 16 to about 60 amino acid residues. In aspects, the XTEN linker includes about 16 to about 50 amino acid residues. In aspects, the XTEN linker includes about 16 to about 40 amino acid residues. In aspects, the XTEN linker includes about 16 to about 35 amino acid residues. In aspects, the XTEN linker includes about 16 to about 30 amino acid residues. In aspects, the XTEN linker includes about 16 to about 25 amino acid residues. In aspects, the XTEN linker includes about 16 to about 20 amino acid residues. In aspects, the XTEN linker includes about 16 amino acid residues. In aspects, the XTEN linker includes about 17 amino acid residues. In aspects, the XTEN linker includes about 18 amino acid residues. In aspects, the XTEN linker includes about 19 amino acid residues. In aspects, the XTEN linker includes about 20 amino acid residues.

In aspects, the fusion protein comprises at least two XTEN linkers that are the same or different. In aspects, the fusion protein comprises a first XTEN linker having more amino acid residues than a second XTEN linker. In aspects, the fusion protein comprises a first XTEN linker having 10 to 150 amino acid residues than a second XTEN linker. In aspects, the fusion protein comprises a first XTEN linker having 20 to 120 amino acid residues than a second XTEN linker. In aspects, the fusion protein comprises a first XTEN linker having 30 to 110 amino acid residues than a second XTEN linker. In aspects, the fusion protein comprises a first XTEN linker having 40 to 110 amino acid residues than a second XTEN linker. In aspects, the fusion protein comprises a first XTEN linker having 50 to 100 amino acid residues than a second XTEN linker. In aspects, the fusion protein comprises a first XTEN linker having 60 to 100 amino acid residues than a second XTEN linker.

In embodiments, the XTEN linker comprises from about 50 to about 864 amino acid residues. In aspects, the XTEN linker comprises from about 50 to about 200 amino acid residues. In aspects, the XTEN linker comprises from about 55 to about 180 amino acid residues. In aspects, the XTEN linker comprises from about 60 to about 150 amino acid residues. In aspects, the XTEN linker comprises from about 60 to about 120 amino acid residues. In aspects, the XTEN linker comprises from about 60 to about 110 amino acid residues. In aspects, the XTEN linker comprises from about 60 to about 100 amino acid residues. In aspects, the XTEN linker comprises from about 70 to about 90 amino acid residues. In aspects, the XTEN linker comprises from about 75 to about 85 amino acid residues. In aspects, the XTEN linker comprises about 80 amino acid residues. In aspects, when a fusion protein comprises at least two XTEN peptide linkers, then the XTEN linker that comprise from about 50 to about 200 amino acid residues is referred to as a first XTEN peptide linker.

In embodiments, the XTEN linker comprises from about 5 to about 55 amino acid residues. In aspects, the XTEN linker comprises from about 5 to about 50 amino acid residues. In aspects, the XTEN linker comprises from about 5 to about 40 amino acid residues. In aspects, the XTEN linker comprises from about 10 to about 30 amino acid residues. In aspects, the XTEN linker comprises from about 10 to about 25 amino acid residues. In aspects, the XTEN linker comprises from about 10 to about 20 amino acid residues. In aspects, the XTEN linker comprises from about 14 to about 18 amino acid residues. In aspects, the XTEN linker comprises about 16 amino acid residues. In aspects, when a fusion protein comprises at least two XTEN peptide linkers, then the XTEN linker that comprise from about 5 to about 55 amino acid residues is referred to as a second XTEN peptide linker.

In embodiments, the XTEN linker includes the sequence set forth by SEQ ID NO:5. In aspects, the XTEN linker is the sequence set forth by SEQ ID NO:5. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:5. In aspects, the XTEN linker has an amino acid sequence that has at least 75% sequence identity to SEQ ID NO:5. In aspects, the XTEN linker has an amino acid sequence that has at least 80% sequence identity to SEQ ID NO:5. In aspects, the XTEN linker has an amino acid sequence that has at least 85% sequence identity to SEQ ID NO:5. In aspects, the XTEN linker has an amino acid sequence that has at least 90% sequence identity to SEQ ID NO:5. In aspects, the XTEN linker has an amino acid sequence that has at least 95% sequence identity to SEQ ID NO:5.

In embodiments, the XTEN linker includes the sequence set forth by SEQ ID NO:6. In aspects, the XTEN linker is the sequence set forth by SEQ ID NO:6. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:6. In aspects, the XTEN linker has an amino acid sequence that has at least 75% sequence identity to SEQ ID NO:6. In aspects, the XTEN linker has an amino acid sequence that has at least 80% sequence identity to SEQ ID NO:6. In aspects, the XTEN linker has an amino acid sequence that has at least 85% sequence identity to SEQ ID NO:6. In aspects, the XTEN linker has an amino acid sequence that has at least 90% sequence identity to SEQ ID NO:6. In aspects, the XTEN linker has an amino acid sequence that has at least 95% sequence identity to SEQ ID NO:6.

In embodiments, the XTEN linker includes the sequence set forth by SEQ ID NO:98. In aspects, the XTEN linker is the sequence set forth by SEQ ID NO:98. In aspects, the XTEN linker has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:98. In aspects, the XTEN linker has an amino acid sequence that has at least 75% sequence identity to SEQ ID NO:98. In aspects, the XTEN linker has an amino acid sequence that has at least 80% sequence identity to SEQ ID NO:98. In aspects, the XTEN linker has an amino acid sequence that has at least 85% sequence identity to SEQ ID NO:98. In aspects, the XTEN linker has an amino acid sequence that has at least 90% sequence identity to SEQ ID NO:98. In aspects, the XTEN linker has an amino acid sequence that has at least 95% sequence identity to SEQ ID NO:98.

The fusion protein may include amino acid sequences useful for targeting the fusion protein to specific regions of a cell (e.g., cytoplasm, nucleus). Thus, in aspects, the fusion protein further includes a nuclear localization signal (NLS) peptide. In aspects, the NLS includes the sequence set forth by SEQ ID NO:4. In aspects, the NLS is the sequence set forth by SEQ ID NO:4. In aspects, the NLS has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:4. In aspects, the NLS has an amino acid sequence that has at least 75% sequence identity to SEQ ID NO:4. In aspects, the NLS has an amino acid sequence that has at least 80% sequence identity to SEQ ID NO:4. In aspects, the NLS has an amino acid sequence that has at least 85% sequence identity to SEQ ID NO:4. In aspects, the NLS has an amino acid sequence that has at least 90% sequence identity to SEQ ID NO:4. In aspects, the NLS has an amino acid sequence that has at least 95% sequence identity to SEQ ID NO:4.

Fusion Proteins

Provided herein are, inter alia, fusion proteins that can be targeted to any locus in the human genome to activate the expression of human genes long term (i.e. inherited through multiple cell divisions), and which can be transiently delivered as mRNA, DNA or RNP. The fusion proteins have multiplex epigenetic editing capabilities for activating transcription, and control transcription by removing epigenetic marks, including methyl groups on nucleobases and repressive histone modifications. The fusion proteins provided herein further comprise multiple domains acting in concert to robustly activate transcription.

In embodiments, the disclosure provides a fusion protein comprising from N-terminus to C-terminus, a demethylation domain, and a nuclease-deficient RNA-guided DNA endonuclease enzyme. In embodiments, the fusion protein comprises from N-terminus to C-terminus, a demethylation domain, an XTEN linker, and a nuclease-deficient RNA-guided DNA endonuclease enzyme. In embodiments, the nuclease-deficient RNA-guided endonuclease enzyme is a CRISPR-associated protein. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In aspects, the fusion protein further comprises a nuclear localization sequence. In aspects, the fusion protein further comprises two or three nuclear localization sequences. In embodiments, the fusion protein has at least 85% sequence identity to the compound of Formula (I): R¹-L¹-R²; wherein R¹ comprises SEQ ID NO:1, SEQ ID NO:2, SEQ ID NO:3, SEQ ID NO:86, or SEQ ID NO:97; L¹ is absent, SEQ ID NO:5, SEQ ID NO:6, or SEQ ID NO:98; and R² comprises SEQ ID NO:9. In embodiments, the fusion protein has at least 90% sequence identity to the compound of Formula (I). In embodiments, the fusion protein has at least 92% sequence identity to the compound of Formula (I). In embodiments, the fusion protein has at least 94% sequence identity to the compound of Formula (I). In embodiments, the fusion protein has at least 95% sequence identity to the compound of Formula (I). In embodiments, the fusion protein has at least 96% sequence identity to the compound of Formula (I). In embodiments, the fusion protein has at least 98% sequence identity to the compound of Formula (I).

In embodiments, the disclosure provides a fusion protein comprising from N-terminus to C-terminus, an RNA-binding sequence, and at least one transcriptional activator. In embodiments, the fusion protein comprises from N-terminus to C-terminus, an RNA-binding sequence, an XTEN linker, and at least one transcriptional activator. In embodiments, the fusion protein comprises from N-terminus to C-terminus, an RNA-binding sequence, an XTEN linker, and at least one transcriptional activator selected from the group consisting of VP64, p65, Rta, or a combination of two or more thereof. In embodiments, the transcriptional activator is VP64, p65, Rta, or a combination of two or more thereof. In embodiments, the transcriptional activator is VP64. In embodiments, the transcriptional activator is p65. In embodiments, the transcriptional activator is Rta. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta. In embodiments, the fusion protein has at least 85% sequence identity to the compound of Formula (II): R⁴-L¹-R³; wherein R⁴ comprises SEQ ID NO:21; L¹ is absent, SEQ ID NO:5, SEQ ID NO:6, or SEQ ID NO:98; and R³ comprises SEQ ID NO:13, SEQ ID NO:14, SEQ ID NO:15, SEQ ID NO:16, SEQ ID NO:17, SEQ ID NO:18, SEQ ID NO:100, or a combination of two or more thereof. In embodiments, R³ comprises SEQ ID NO:14, SEQ ID NO:15, SEQ ID NO:17, SEQ ID NO:100, or a combination of two or more thereof. In embodiments, the fusion protein has at least 90% sequence identity to the compound of Formula (II). In embodiments, the fusion protein has at least 92% sequence identity to the compound of Formula (II). In embodiments, the fusion protein has at least 94% sequence identity to the compound of Formula (II). In embodiments, the fusion protein has at least 95% sequence identity to the compound of Formula (II). In embodiments, the fusion protein has at least 96% sequence identity to the compound of Formula (II). In embodiments, the fusion protein has at least 98% sequence identity to the compound of Formula (III).

In embodiments, the fusion protein having from N-terminus to C-terminus, an RNA-binding sequence, an XTEN linker, and at least one transcriptional activator comprises SEQ ID NO:104, SEQ ID NO:105, SEQ ID NO:106, SEQ ID NO:107, SEQ ID NO:108, SEQ ID NO:109, or SEQ ID NO:110. In aspects, the fusion protein comprises an amino acid sequence having at least 80% sequence identity to SEQ ID NO:104, SEQ ID NO:105, SEQ ID NO:106, SEQ ID NO:107, SEQ ID NO:108, SEQ ID NO:109, or SEQ ID NO:110. In aspects, the fusion protein comprises an amino acid sequence having at least 85% sequence identity to SEQ ID NO:104, SEQ ID NO:105, SEQ ID NO:106, SEQ ID NO:107, SEQ ID NO:108, SEQ ID NO:109, or SEQ ID NO:110. In aspects, the fusion protein comprises an amino acid sequence having at least 90% sequence identity to SEQ ID NO:104, SEQ ID NO:105, SEQ ID NO:106, SEQ ID NO:107, SEQ ID NO:108, SEQ ID NO:109, or SEQ ID NO:110. In aspects, the fusion protein comprises an amino acid sequence having at least 95% sequence identity to SEQ ID NO:104, SEQ ID NO:105, SEQ ID NO:106, SEQ ID NO:107, SEQ ID NO:108, SEQ ID NO:109, or SEQ ID NO:110.

In embodiments, the disclosure provides a fusion protein comprising from N-terminus to C-terminus, a demethylation domain, a nuclease-deficient RNA-guided DNA endonuclease enzyme, and a transcriptional activator. In embodiments, the fusion protein comprises from N-terminus to C-terminus, a demethylation domain, an XTEN linker, a nuclease-deficient RNA-guided DNA endonuclease enzyme, and a transcriptional activator. In embodiments, the nuclease-deficient RNA-guided endonuclease enzyme is a CRISPR-associated protein. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In embodiments, the transcriptional activator comprises VP64, p65, Rta, or a combination of two or more thereof. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta. In aspects, the fusion protein further comprises a nuclear localization sequence. In aspects, the fusion protein further comprises two or three nuclear localization sequences. In embodiments, the fusion protein has at least 85% sequence identity to the compound of Formula (III): R¹-L¹-R²-R³; wherein R¹ comprises SEQ ID NO:1, SEQ ID NO:2, SEQ ID NO:3, SEQ ID NO:86, SEQ ID NO:97; L¹ is absent, SEQ ID NO:5, SEQ ID NO:6, or SEQ ID NO:98; R² comprises SEQ ID NO:9; and R³ comprises SEQ ID NO:13, SEQ ID NO:14, SEQ ID NO:15, SEQ ID NO:16, SEQ ID NO:17, SEQ ID NO:18, SEQ ID NO:100, or a combination of two or more thereof. In embodiments, R³ comprises SEQ ID NO:14, SEQ ID NO:15, SEQ ID NO:17, SEQ ID NO:100, or a combination of two or more thereof. In embodiments, the fusion protein has at least 90% sequence identity to the compound of Formula (III). In embodiments, the fusion protein has at least 92% sequence identity to the compound of Formula (III). In embodiments, the fusion protein has at least 94% sequence identity to the compound of Formula (III). In embodiments, the fusion protein has at least 95% sequence identity to the compound of Formula (III). In embodiments, the fusion protein has at least 96% sequence identity to the compound of Formula (III). In embodiments, the fusion protein has at least 98% sequence identity to the compound of Formula (III).

In embodiments, the fusion protein comprising from N-terminus to C-terminus, a demethylation domain, an XTEN linker, and a nuclease-deficient RNA-guided DNA endonuclease enzyme. In embodiments, the nuclease-deficient RNA-guided endonuclease enzyme is a CRISPR-associated protein. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In embodiments, the fusion protein further comprises a transcriptional activator. In embodiments, the transcriptional activator comprises VP64, p65, Rta, or a combination of two or more thereof. In aspects, the fusion protein further comprises a nuclear localization sequence. In aspects, the fusion protein further comprises two or three nuclear localization sequences.

In embodiments, the nuclease-deficient RNA-guided DNA endonuclease enzyme is dCas9, dCas12a, dCpf1, a zinc finger domain, a leucine zipper domain, a winged helix domain, TALE, a helix-turn-helix motif, a helix-loop-helix domain, an HMB-box domain, a Wor3 domain, an OB-fold domain, an immunoglobulin domain, or a B3 domain. In embodiments, the nuclease-deficient RNA-guided DNA endonuclease enzyme is a CRISPR-associated protein. In embodiments, the nuclease-deficient RNA-guided DNA endonuclease enzyme is dCas9. In embodiments, the nuclease-deficient RNA-guided DNA endonuclease enzyme is dCpf1. In embodiments, the nuclease-deficient RNA-guided DNA endonuclease enzyme is Cas-phi. In embodiments, the nuclease-deficient RNA-guided DNA endonuclease enzyme is a leucine zipper domain. In embodiments, the nuclease-deficient RNA-guided DNA endonuclease enzyme is a winged helix domain. In embodiments, the nuclease-deficient RNA-guided DNA endonuclease enzyme is a helix-turn-helix motif. In embodiments, the nuclease-deficient RNA-guided DNA endonuclease enzyme is a helix-loop-helix domain. In embodiments, the nuclease-deficient RNA-guided DNA endonuclease enzyme is an HMB-box domain. In embodiments, the nuclease-deficient RNA-guided DNA endonuclease enzyme is a Wor3 domain. In embodiments, the nuclease-deficient RNA-guided DNA endonuclease enzyme is an OB-fold domain. In embodiments, the nuclease-deficient RNA-guided DNA endonuclease enzyme is an immunoglobulin domain. In embodiments, the nuclease-deficient RNA-guided DNA endonuclease enzyme is a B3 domain.

In embodiments, the fusion protein comprising from N-terminus to C-terminus, a demethylation domain, an XTEN linker, and a nuclease-deficient DNA endonuclease enzyme. In embodiments, the nuclease-deficient endonuclease enzyme is a zinc finger domain. In embodiments, the nuclease-deficient endonuclease enzyme is a TALE. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In aspects, the fusion protein further comprises a nuclear localization sequence. In aspects, the fusion protein further comprises two or three nuclear localization sequences.

In embodiments, the fusion protein comprising from N-terminus to C-terminus, a demethylation domain, an XTEN linker, a nuclease-deficient DNA endonuclease enzyme, and a transcriptional activator. In embodiments, the nuclease-deficient endonuclease enzyme is a zinc finger domain. In embodiments, the nuclease-deficient endonuclease enzyme is a TALE. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In embodiments, the transcriptional activator comprises VP64, p65, Rta, or a combination of two or more thereof. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta. In aspects, the fusion protein further comprises a nuclear localization sequence. In aspects, the fusion protein further comprises two or three nuclear localization sequences.

In embodiments, the fusion protein comprising from N-terminus to C-terminus, a demethylation domain, an XTEN linker, and a nuclease-deficient DNA endonuclease enzyme. In embodiments, the nuclease-deficient endonuclease enzyme is a zinc finger domain. In embodiments, the nuclease-deficient endonuclease enzyme is a TALE. In embodiments, the demethylation domain is a TET1 domain, a TET2 domain, a TET3 domain, or a combination of two or more thereof. In embodiments, the demethylation domain is a TET1 domain. In embodiments, the demethylation domain is a TET2 domain. In embodiments, the demethylation domain is a TET3 domain. In embodiments, the fusion protein further comprises a transcriptional activator. In embodiments, the transcriptional activator comprises VP64, p65, Rta, or a combination of two or more thereof. In aspects, the fusion protein further comprises a nuclear localization sequence. In aspects, the fusion protein further comprises two or three nuclear localization sequences.

In embodiments, the XTEN linker comprises from about 5 to about 864 amino acid residues. In aspects, the XTEN linker comprises from about 10 to about 864 amino acid residues. In aspects, the XTEN linker comprises from about 20 to about 864 amino acid residues. In aspects, the XTEN linker comprises from about 30 to about 864 amino acid residues. In aspects, the XTEN linker comprises from about 40 to about 864 amino acid residues. In aspects, the XTEN linker comprises from about 50 to about 200 amino acid residues. In aspects, the XTEN linker comprises from about 55 to about 180 amino acid residues. In aspects, the XTEN linker comprises from about 60 to about 150 amino acid residues. In aspects, the XTEN linker comprises from about 60 to about 120 amino acid residues. In aspects, the XTEN linker comprises from about 60 to about 110 amino acid residues. In aspects, the XTEN linker comprises from about 60 to about 100 amino acid residues. In aspects, the XTEN linker comprises from about 70 to about 90 amino acid residues. In aspects, the XTEN linker comprises from about 75 to about 85 amino acid residues. In aspects, the XTEN linker comprises about 80 amino acid residues. In aspects, when a fusion protein comprises at least two XTEN peptide linkers, then the XTEN linker that comprise from about 50 to about 200 amino acid residues is referred to as a first XTEN peptide linker.

In embodiments, the XTEN linker comprises from about 5 to about 55 amino acid residues. In aspects, the XTEN linker comprises from about 5 to about 50 amino acid residues. In aspects, the XTEN linker comprises from about 5 to about 40 amino acid residues. In aspects, the XTEN linker comprises from about 10 to about 30 amino acid residues. In aspects, the XTEN linker comprises from about 10 to about 25 amino acid residues. In aspects, the XTEN linker comprises from about 10 to about 20 amino acid residues. In aspects, the XTEN linker comprises from about 14 to about 18 amino acid residues. In aspects, the XTEN linker comprises about 16 amino acid residues. In aspects, when a fusion protein comprises at least two XTEN peptide linkers, then the XTEN linker that comprise from about 5 to about 55 amino acid residues is referred to as a second XTEN peptide linker.

For the fusion protein provided herein, in embodiments, the fusion protein further comprises an epitope tag, a 2A peptide, a fluorescent protein tag, a nuclear localization signal peptide, or a combination of two or more thereof. In embodiments, the fusion protein further comprises an epitope tag. In embodiments, the fusion protein further comprises a 2A peptide. In embodiments, the fusion protein further comprises a fluorescent protein tag. In embodiments, the fusion protein further comprises a nuclear localization signal peptide.

For the fusion protein provided herein, in embodiments, the fusion protein further comprises at least one transcriptional activator. In embodiments, the transcriptional activator comprises VP64, p65, Rta, or a combination of two or more thereof. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta.

In embodiments, the RNA-binding sequence is an MS2 RNA-binding sequence. In embodiments, the MS2 RNA-binding sequence comprises MCP protein.

The fusion protein may comprise an XTEN linker as described herein. In embodiments, the XTEN linker comprises from about 10 amino acid residues to about 864 amino acid residues.

In embodiments, the fusion protein comprises, from N-terminus to C-terminus, a TET1 domain, an XTEN linker, a CRISPR-associated protein, an XTEN linker, a nuclear localization sequence, a transcriptional activator, and a nuclear localization sequence. In embodiments, the fusion protein comprises, from N-terminus to C-terminus, a TET1 domain, an XTEN linker, a zinc finger domain, an XTEN linker, a nuclear localization sequence, a transcriptional activator, and a nuclear localization sequence. In embodiments, the fusion protein comprises, from N-terminus to C-terminus, a TET1 domain, an XTEN linker, a TALE, an XTEN linker, a nuclear localization sequence, Rta, and a nuclear localization sequence. In embodiments, the fusion protein comprises, from N-terminus to C-terminus, a TET1 domain, an XTEN linker, dCas9, an XTEN linker, a nuclear localization sequence, a transcriptional activator, and a nuclear localization sequence. In embodiments, the transcriptional activator comprises VP64, p65, Rta, or a combination of two or more thereof. In embodiments, the transcriptional activator comprises Rta. In embodiments, the transcriptional activator comprises VP64. In embodiments, the transcriptional activator comprises p65. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta. In embodiments, the fusion protein comprises, from N-terminus to C-terminus, SEQ ID NO:97, SEQ ID NO:98, SEQ ID NO:9, SEQ ID NO:6, SEQ ID NO:4, SEQ ID NO:15, and SEQ ID NO:4. In embodiments, the fusion protein comprises SEQ ID NO:99. In embodiments, the fusion protein is SEQ ID NO:99. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:99. In aspects, the fusion protein has an amino acid sequence that has at least 75% sequence identity to SEQ ID NO:99. In aspects, the fusion protein has an amino acid sequence that has at least 80% sequence identity to SEQ ID NO:99. In aspects, the fusion protein has an amino acid sequence that has at least 85% sequence identity to SEQ ID NO:99. In aspects, the fusion protein has an amino acid sequence that has at least 90% sequence identity to SEQ ID NO:99. In aspects, the fusion protein has an amino acid sequence that has at least 95% sequence identity to SEQ ID NO:99.

In embodiments, the fusion protein comprises, from N-terminus to C-terminus, a TET1 domain, an XTEN linker, a CRISPR-associated protein, an XTEN linker, a nuclear localization sequence, two transcriptional activators, and a nuclear localization sequence. In embodiments, the fusion protein comprises, from N-terminus to C-terminus, a TET1 domain, an XTEN linker, a zinc finger domain, an XTEN linker, a nuclear localization sequence, p65, Rta, and a nuclear localization sequence. In embodiments, the fusion protein comprises, from N-terminus to C-terminus, a TET1 domain, an XTEN linker, a TALE, an XTEN linker, a nuclear localization sequence, two transcriptional activators, and a nuclear localization sequence. In embodiments, the fusion protein comprises, from N-terminus to C-terminus, a TET1 domain, an XTEN linker, dCas9, an XTEN linker, a nuclear localization sequence, two transcriptional activators, and a nuclear localization sequence. In embodiments, the transcriptional activator comprises at least two of VP64, p65, and Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta. In embodiments, the fusion protein comprises, from N-terminus to C-terminus, SEQ ID NO:97, SEQ ID NO:98, SEQ ID NO:9, SEQ ID NO:6, SEQ ID NO:4, SEQ ID NO:100, SEQ ID NO:15, and SEQ ID NO:4. In embodiments, the fusion protein comprises SEQ ID NO:101. In embodiments, the fusion protein is SEQ ID NO:101. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:101. In aspects, the fusion protein has an amino acid sequence that has at least 75% sequence identity to SEQ ID NO:101. In aspects, the fusion protein has an amino acid sequence that has at least 80% sequence identity to SEQ ID NO:101. In aspects, the fusion protein has an amino acid sequence that has at least 85% sequence identity to SEQ ID NO:101. In aspects, the fusion protein has an amino acid sequence that has at least 90% sequence identity to SEQ ID NO:101. In aspects, the fusion protein has an amino acid sequence that has at least 95% sequence identity to SEQ ID NO:101.

In embodiments, the fusion protein comprises, from N-terminus to C-terminus, a TET1 domain, an XTEN linker, a CAS-associated protein, and from 1 to 3 nuclear localization sequences. In embodiments, the fusion protein comprises, from N-terminus to C-terminus, a TET1 domain, an XTEN linker, a zinc finger domain, and from 1 to 3 nuclear localization sequences. In embodiments, the fusion protein comprises, from N-terminus to C-terminus, a TET1 domain, an XTEN linker, a TALE, and from 1 to 3 nuclear localization sequences. In embodiments, the fusion protein comprises, from N-terminus to C-terminus, a TET1 domain, an XTEN linker, dCas9, and from 1 to 3 nuclear localization sequences. In embodiments, the fusion protein further comprises a transcriptional activator. In embodiments, the fusion protein comprises, from N-terminus to C-terminus, SEQ ID NO:97, SEQ ID NO:98, SEQ ID NO:9, and SEQ ID NO:4. In embodiments, the fusion protein comprises SEQ ID NO:102. In embodiments, the fusion protein is SEQ ID NO:102. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:102. In aspects, the fusion protein has an amino acid sequence that has at least 75% sequence identity to SEQ ID NO:102. In aspects, the fusion protein has an amino acid sequence that has at least 80% sequence identity to SEQ ID NO:102. In aspects, the fusion protein has an amino acid sequence that has at least 85% sequence identity to SEQ ID NO:102. In aspects, the fusion protein has an amino acid sequence that has at least 90% sequence identity to SEQ ID NO:102. In aspects, the fusion protein has an amino acid sequence that has at least 95% sequence identity to SEQ ID NO:102.

In embodiments, the fusion protein comprises SEQ ID NO:103. In embodiments, the fusion protein is SEQ ID NO:103. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:103. In aspects, the fusion protein has an amino acid sequence that has at least 75% sequence identity to SEQ ID NO:103. In aspects, the fusion protein has an amino acid sequence that has at least 80% sequence identity to SEQ ID NO:103. In aspects, the fusion protein has an amino acid sequence that has at least 85% sequence identity to SEQ ID NO:103. In aspects, the fusion protein has an amino acid sequence that has at least 90% sequence identity to SEQ ID NO:103. In aspects, the fusion protein has an amino acid sequence that has at least 95% sequence identity to SEQ ID NO:103.

In embodiments, the fusion protein comprises SEQ ID NO:111. In embodiments, the fusion protein is SEQ ID NO:111. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:111. In aspects, the fusion protein has an amino acid sequence that has at least 75% sequence identity to SEQ ID NO:111. In aspects, the fusion protein has an amino acid sequence that has at least 80% sequence identity to SEQ ID NO:111. In aspects, the fusion protein has an amino acid sequence that has at least 85% sequence identity to SEQ ID NO:111. In aspects, the fusion protein has an amino acid sequence that has at least 90% sequence identity to SEQ ID NO:111. In aspects, the fusion protein has an amino acid sequence that has at least 95% sequence identity to SEQ ID NO:111.

In embodiments, the fusion protein comprises SEQ ID NO:112. In embodiments, the fusion protein is SEQ ID NO:112. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:112. In aspects, the fusion protein has an amino acid sequence that has at least 75% sequence identity to SEQ ID NO:112. In aspects, the fusion protein has an amino acid sequence that has at least 80% sequence identity to SEQ ID NO:112. In aspects, the fusion protein has an amino acid sequence that has at least 85% sequence identity to SEQ ID NO:112. In aspects, the fusion protein has an amino acid sequence that has at least 90% sequence identity to SEQ ID NO:112. In aspects, the fusion protein has an amino acid sequence that has at least 95% sequence identity to SEQ ID NO:112.

In embodiments, the fusion protein comprises SEQ ID NO:113. In embodiments, the fusion protein is SEQ ID NO:113. In aspects, the fusion protein has an amino acid sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:113. In aspects, the fusion protein has an amino acid sequence that has at least 75% sequence identity to SEQ ID NO:113. In aspects, the fusion protein has an amino acid sequence that has at least 80% sequence identity to SEQ ID NO:113. In aspects, the fusion protein has an amino acid sequence that has at least 85% sequence identity to SEQ ID NO:113. In aspects, the fusion protein has an amino acid sequence that has at least 90% sequence identity to SEQ ID NO:113. In aspects, the fusion protein has an amino acid sequence that has at least 95% sequence identity to SEQ ID NO:113.

Provided herein are compounds of Formula (III) or compounds having at least 85% sequence identity to the compound of Formula (III), wherein the compound of Formula (III) is R¹⁰-L¹-R¹¹-R¹²-L²-L³-(R¹³-L⁴)ₓR¹⁴-X¹-L⁵-X²-L⁶-X³-L⁷-R¹⁵. In embodiments, the compounds have at least 90% sequence identity to the compound of Formula (III). In embodiments, the compounds have at least 92% sequence identity to the compound of Formula (III). In embodiments, the compounds have at least 94% sequence identity to the compound of Formula (III). In embodiments, the compounds have at least 95% sequence identity to the compound of Formula (III). In embodiments, the compounds have at least 96% sequence identity to the compound of Formula (III). In embodiments, the compounds have at least 98% sequence identity to the compound of Formula (III). In embodiments, the compounds are of Formula (III). R¹⁰ is a demethylation domain. In embodiments R¹⁰ comprises SEQ ID NO:1, 2, 3, 86, 97 (including embodiments thereof). In embodiments R¹⁰ comprises SEQ ID NO:97 (including embodiments thereof). L¹ is a bond or a peptide linker. In embodiments, L¹ is a bond. R¹¹ is an XTEN linker. In embodiments, R¹¹ comprises SEQ ID NO:5, 6, or 98 (including embodiments thereof. In embodiments, R¹¹ comprises SEQ ID NO:5 (including embodiments thereof. In embodiments, R¹¹ comprises SEQ ID NO:6 (including embodiments thereof. In embodiments, R¹¹ comprises SEQ ID NO:98 (including embodiments thereof). R¹² comprises a nuclease-deficient RNA-guided DNA endonuclease enzyme or a nuclease-deficient endonuclease enzyme. In embodiments, R¹² comprises a nuclease-deficient RNA-guided DNA endonuclease enzyme. In embodiments, R¹² comprises a CRISPR-associated protein. In embodiments, R¹² comprises SEQ ID NO:9 (including embodiments thereof). In embodiments, R¹² comprises a nuclease-deficient endonuclease enzyme. In embodiments, R¹² comprises a zinc finger domain or a TALE. In embodiments, R¹² comprises a zinc finger domain. In embodiments, R¹² comprises a TALE. L² is a bond or an XTEN linker. In embodiments, L² is a bond or an XTEN linker. In embodiments, L² is a bond. In embodiments, L² is an XTEN linker. In embodiments, L² comprises SEQ ID NO:5, 6, or 98 (including embodiments thereof). In embodiments, L² comprises SEQ ID NO:5 (including embodiments thereof. In embodiments, L² comprises SEQ ID NO:6 (including embodiments thereof. In embodiments, L² comprises SEQ ID NO:98 (including embodiments thereof). L³ is a bond or a peptide linker. In embodiments, L³ is a bond. In embodiments, L³ is a peptide linker. In embodiments, L³ is a peptide linker comprising from 1 amino acid to about 10 amino acids. In embodiments, L³ is a peptide linker comprising from 3 amino acids to about 5 amino acids. R¹³ comprises a nuclear localization sequence. In embodiments, R¹³ comprises SEQ ID NO:4 (including embodiments thereof). L⁴ is absent or a peptide linker. In embodiments, L⁴ is absent. In embodiments, L⁴ is a peptide linker. In embodiments, L⁴ is a peptide linker comprising from 1 amino acid to about 10 amino acids. In embodiments, L⁴ is a peptide linker comprising from 1 amino acid to about 5 amino acids. In embodiments, L⁴ is a peptide linker comprising 1 amino acid to about 4 amino acids. x is an integer from 0 to 4. In embodiments, x is 0. In embodiments, x is 1. In embodiments, x is 2. In embodiments, x is 3. R¹⁴ is absent or a nuclear localization sequence. In embodiments, R¹⁴ is absent. In embodiments, R¹⁴ is a nuclear localization sequence. In embodiments, R¹⁴ comprises SEQ ID NO:4 (including embodiments thereof). X¹, X², and X³ are independently absent or a transcriptional activator. In embodiments, X¹, X², and X³ are independently a transcriptional activator. In embodiments, X¹, X², and X³ are independently p65, Rta, or VP64. In embodiments, X¹, X², and X³ are independently p65, Rta, or VP64, wherein each of X¹, X², and X³ are different. In embodiments, X¹ and X² are independently p65, Rta, or VP64, and X³ is absent. In embodiments, X¹ and X² are independently p65, Rta, or VP64; X³ is absent; and X¹ and X² are different. In embodiments, X¹ is p65, Rta, or VP64; X² is absent; and X³ is absent. In embodiments, p65 comprises SEQ ID NO:13, 14, or 100 (including embodiments thereof). In embodiments, p65 comprises SEQ ID NO:13 (including embodiments thereof). In embodiments, p65 comprises SEQ ID NO:14 (including embodiments thereof). In embodiments, p65 comprises SEQ ID NO:100 (including embodiments thereof). In embodiments, Rta comprises SEQ ID NO:15 or 16 (including embodiments thereof). In embodiments, Rta comprises SEQ ID NO:15 (including embodiments thereof). In embodiments, Rta comprises SEQ ID NO:16 (including embodiments thereof). In embodiments, VP64 comprises SEQ ID NO:17 or 18 (including embodiments thereof). In embodiments, VP64 comprises SEQ ID NO:17 (including embodiments thereof). In embodiments, VP64 comprises SEQ ID NO:18 (including embodiments thereof). L⁵ is absent or a peptide linker. In embodiments, L⁵ is absent. In embodiments, L⁵ comprises a peptide linker. In embodiments, peptide linker comprises from 1 amino acid to about 10 amino acids. In embodiments, the peptide linker comprises from 3 amino acids to about 5 amino acids. L⁶ is absent or a peptide linker. In embodiments, L⁶ is absent. In embodiments, L⁶ comprises a peptide linker. In embodiments, peptide linker comprises from 1 amino acid to about 10 amino acids. In embodiments, the peptide linker comprises from 3 amino acids to about 5 amino acids. L⁷ is absent or a peptide linker. In embodiments, L⁷ is absent. In embodiments, L⁷ comprises a peptide linker. In embodiments, peptide linker comprises from 1 amino acid to about 10 amino acids. In embodiments, the peptide linker comprises from 3 amino acids to about 5 amino acids. In embodiments, when X¹ is absent, then L⁵ is absent. In embodiments, when X² is absent, then L⁶ is absent. In embodiments, when X³ is absent, then L⁷ is absent. In embodiments, when X² is absent, then X³ is absent, and L⁶ and L⁷ are absent. In embodiments, when X¹ is absent, then X² and X³ are absent, and L⁵, L⁶, and L⁷ are absent. R¹⁵ is absent or a nuclear localization sequence. In embodiments, R¹⁵ is absent. In embodiments, R¹⁵ is a nuclear localization sequence. In embodiments, R¹⁵ comprises SEQ ID NO:4 (including embodiments thereof).

In the sequences listed herein, the skilled artisan will appreciate that a methionine (M) can be present on the N-terminus of the protein in order to initiate translation. Thus, the sequences described herein can optionally further comprise a methionone on the N-terminus.

Complexes

In order for the fusion protein to carry out epigenome editing, the fusion protein interacts with (e.g. is non-covalently bound to) a polynucleotide (e.g., sgRNA) that is complementary to a target polynucleotide sequence (e.g., a target DNA sequence to be edited) and further includes a sequence (i.e., a binding sequence) to which the nuclease-deficient RNA-guided DNA endonuclease enzyme of the fusion protein as described herein can bind. In aspects, the polynucleotide that is complementary to a target polynucleotide sequence (e.g., a target genomic DNA sequence to be edited) and further includes a binding sequence to which the nuclease-deficient RNA-guided DNA endonuclease enzyme of the fusion protein as described herein can bind is sgRNA. In aspects, the polynucleotide that is complementary to a target polynucleotide sequence (e.g., a target DNA sequence to be edited) and further includes a binding sequence to which the nuclease-deficient RNA-guided DNA endonuclease enzyme of the fusion protein as described herein can bind is cr:tracrRNA. By forming this complex, the fusion protein is appropriately positioned to perform epigenome editing. The term “complex” refers to a composition that includes two or more components, where the components bind together to make a functional unit. In aspects, a complex described herein includes a fusion protein described herein and a polynucleotide described herein. Thus, in an aspect is provided a fusion protein as described herein, including embodiments and aspects thereof, and sgRNA or cr:tracrRNA (i.e., a polynucleotide including: (1) a DNA-targeting sequence that is complementary to a target polynucleotide sequence; and (2) a binding sequence for the nuclease-deficient RNA-guided DNA endonuclease enzyme, wherein the nuclease-deficient RNA-guided DNA endonuclease enzyme is bound to the polynucleotide via the binding sequence (e.g., an amino acid sequence capable of binding to the DNA-targeting sequence)). In aspects the polynucleotide comprises at least one MS2 loop.

In aspects, a complex described herein includes a fusion protein described herein, a polynucleotide described herein, and a second fusion protein described herein. In aspects, the second fusion protein comprises a transcriptional activator described herein.

A DNA-targeting sequence refers to a polynucleotide that includes a nucleotide sequence complementary to the target polynucleotide sequence (DNA or RNA). In aspects, a DNA-targeting sequence can be a single RNA molecule (single RNA polynucleotide), which may include a “single-guide RNA,” or “sgRNA.” In aspects, the DNA-targeting sequence includes two RNA molecules (e.g., two sgRNA), referred to as a guide RNA (gRNA) (e.g., joined together via hybridization at the binding sequence (e.g., dCas9-binding sequence). In aspects, the DNA-targeting sequence (e.g., sgRNA) is at least 50%, 55%, 60%, 65%, 70%, 75%, 80%, 85%, 90%, 95%, 96%, 97%, 98% or 99% complementary to the target polynucleotide sequence. In aspects, the DNA-targeting sequence (e.g., sgRNA) is at least 50%, 55%, 60%, 65%, 70%, 75%, 80%, 85%, 90%, 95%, 96%, 97%, 98% or 99% complementary to the sequence of a cellular gene. In aspects, the DNA-targeting sequence (e.g., sgRNA) binds a cellular gene sequence. In aspects, the DNA-targeting sequence (e.g., sgRNA) is at least 75% complementary to the sequence of a cellular gene. In aspects, the DNA-targeting sequence (e.g., sgRNA) is at least 80% complementary to the sequence of a cellular gene. In aspects, the DNA-targeting sequence (e.g., sgRNA) binds a cellular gene sequence. In aspects, the DNA-targeting sequence (e.g., sgRNA) is at least 85% complementary to the sequence of a cellular gene. In aspects, the DNA-targeting sequence (e.g., sgRNA) binds a cellular gene sequence. In aspects, the DNA-targeting sequence (e.g., sgRNA) is at least 90% complementary to the sequence of a cellular gene. In aspects, the DNA-targeting sequence (e.g., sgRNA) binds a cellular gene sequence. In aspects, the DNA-targeting sequence (e.g., sgRNA) is at least 95% complementary to the sequence of a cellular gene. In aspects, the DNA-targeting sequence (e.g., sgRNA) binds a cellular gene sequence. In aspects, the DNA-targeting sequence (e.g., sgRNA) comprises at least one MS2 stem loop. In embodiments, the MS2 stem loop comprises the sequence of SEQ ID NO:19. In embodiments, the MS2 stem loop has the sequence of SEQ ID NO:19. In aspects, the MS2 stem loop has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:19.

A “target polynucleotide sequence” as provided herein is a nucleic acid sequence present in, or expressed by, a cell, to which a guide sequence (or a DNA-targeting sequence) is designed to have complementarity, where hybridization between a target sequence and a guide sequence (or a DNA-targeting sequence) promotes the formation of a complex (e.g., CRISPR complex). Full complementarity is not necessarily required, provided there is sufficient complementarity to cause hybridization and promote formation of a complex (e.g., CRISPR complex). In aspects, the target polynucleotide sequence is an exogenous nucleic acid sequence. In aspects, the target polynucleotide sequence is an endogenous nucleic acid sequence.

The target polynucleotide sequence may be any region of the polynucleotide (e.g., DNA sequence) suitable for epigenome editing. In aspects, the target polynucleotide sequence is part of a gene. In aspects, the target polynucleotide sequence is part of a transcriptional regulatory sequence. In aspects, the target polynucleotide sequence is part of a promoter, enhancer or silencer. In aspects, the target polynucleotide sequence is part of a promoter. In aspects, the target polynucleotide sequence is part of an enhancer. In aspects, the target polynucleotide sequence is part of a silencer.

In embodiments, the target polynucleotide sequence is a hypermethylated nucleic acid sequence. A “hypermethylated nucleic acid sequence” is used herein according to the standard meaning in the art and refers to frequent methylation of cytosine to 5-methylcytosine (e.g., in CpG). The frequency or occurrence of methyl groups may be relative to a standard control. Hypermethylation may occur, for example, in cancer (e.g., in DNA repair or apoptosis pathways) relative to the non-cancer cell, respectively. Thus, the complex may be useful for reestablishing normal (e.g. non-diseased) methylation levels.

In embodiments, the target polynucleotide sequence is within or adjacent to a transcription start site. In aspects, the target polynucleotide sequence is within about 3000, 2500, 2000, 1500, 500, 100, 80, 70, 60, 50, 40, 30, 20, 10, or fewer base pairs (bp) flanking a transcription start site.

In embodiments, the target polynucleotide sequence is at, near, or within a promoter sequence. In aspects, the target polynucleotide sequence is within a CpG island. In aspects, the target polynucleotide sequence is within a non-CpG island. In aspects, the target polynucleotide sequence is known to be associated with a disease or condition characterized by DNA hypermethylation or hypomethylation.

In embodiments, the complex includes dCas9 bound to the polynucleotide through binding a binding sequence of the polynucleotide and thereby forming a ribonucleoprotein complex. In aspects, the binding sequence forms a hairpin structure. In aspects, the binding sequence is 10-200 nt, 15-150 nt, 20-140 nt, 30-100 nt in length.

In embodiments, the binding sequence (e.g., Cas9-binding sequence) interacts with or binds to a Cas9 protein (e.g., dCas9 protein), and together they bind to the target polynucleotide sequence recognized by the DNA-targeting sequence. The binding sequence (e.g., Cas9-binding sequence) includes two complementary stretches of nucleotides that hybridize to one another to form a double stranded RNA duplex (a dsRNA duplex). These two complementary stretches of nucleotides may be covalently linked by intervening nucleotides known as linkers or linker nucleotides (e.g., in the case of a single-molecule polynucleotide), and hybridize to form the double stranded RNA duplex (dsRNA duplex, or “Cas9-binding hairpin”) of the binding sequence (e.g., Cas9-binding sequence), thus resulting in a stem-loop structure. Alternatively, in some aspects, the two complementary stretches of nucleotides may not be covalently linked, but instead are held together by hybridization between complementary sequences (e.g., a two-molecule polynucleotide).

The binding sequence (e.g., Cas9-binding sequence) can have a length of from 10 nucleotides to 200 nucleotides, e.g., from 20 nucleotides (nt) to 150 nt. In aspects, the binding sequence has a length of from 80 nucleotides (nt) to 100 nt. The dsRNA duplex of the binding sequence (e.g., Cas9-binding sequence) can have a length from 6 base pairs (bp) to 200 bp. For example, the dsRNA duplex of the binding sequence (e.g., Cas9-binding sequence) can have a length from 6 bp to 200 bp, from 10 bp to 180 bp, from 10 bp to 150 bp, from 80 bp to 100 bp, and the like.

Nucleic Acids and Vectors

The fusion protein described herein, including embodiments thereof, may be delivered to the cell in a variety of methods known in the art. The fusion protein may be expressed transiently, bypassing the necessity of viral delivery methods. The fusion protein may be encoded on RNA or DNA delivered to cells as a modified or unmodified RNA or plasmid DNA. The RNA or DNA encoding the protein may be delivered by transfection, lipid nanoparticle, virus like particle (VLP) or virus. In theory, the protein may also be directly delivered via transfection or lipid nanoparticle or VLP.

The fusion protein described herein, including embodiments and aspects thereof, may be provided as a nucleic acid sequence that encodes for the fusion protein. Thus, in an aspect is provided a nucleic acid sequence encoding the fusion protein described herein, including embodiments and aspects thereof. In an aspect is provided a nucleic acid sequence encoding the fusion protein described herein (including the DNA-targeting sequence), including embodiments and aspects thereof. In aspects, the nucleic acid sequence encodes for a fusion protein described herein, including fusion proteins having amino acid sequences with certain % sequence identities described herein. In aspects, the nucleic acid is RNA. In aspects, the nucleic acid is messenger RNA. In aspects, fusion protein is delivered as DNA, mRNA, protein or an RNP. For RNP the protein would be dCas9 and the RNA would encode an sgRNA. Similarly the sgRNA could be delivered as DNA encoding a promoter and an sgRNA, RNA encoding a promoter and an sgRNA. In aspects, the nucleic acid sequence encodes for the fusion proteins described herein, including embodiments and aspects thereof.

In aspects, the fusion proteins and sgRNA or cr:tracrRNA provided herein including embodiments thereof may be provided as a single nucleic acid that encodes for the fusion protein and sgRNA or cr:tracrRNA. In aspects, the fusion proteins and sgRNA or cr:tracrRNA provided herein including embodiments thereof may be provided as multiple nucleic acids that encode for the fusion protein and sgRNA or cr:tracrRNA. In embodiments, the fusion protein and sgRNA or cr:tracrRNA are provided as separate transcripts.

In an aspect is provided a nucleic acid encoding a fusion protein comprising a demethylation domain, an XTEN linker, and a nuclease-deficient RNA-guided DNA endonuclease enzyme.

In an aspect is provided a second nucleic acid encoding an sgRNA or a cr:tracrRNA. In embodiments, the sgRNA comprises at least one MS2 sequence. In embodiments, the sgRNA comprises two MS2 sequences. In embodiments, the second nucleic acid sequence further encodes an MS2-RNA binding sequence, and at least one transcriptional activator provided herein.

In an aspect is provided a third nucleic acid encoding a transcriptional activator. In embodiments, the third nucleic acid further encodes an RNA-binding sequence and an XTEN linker. In embodiments, the RNA-binding sequence is an MS2 RNA-binding sequence.

It is further contemplated that the nucleic acid sequence encoding the fusion protein as described herein, including embodiments and aspects thereof, may be included in a vector. Therefore, in an aspect is provided a vector including a nucleic acid sequence as described herein, including embodiments and aspects thereof. In aspects, the vector comprises a nucleic acid sequence that encodes for a fusion protein described herein, including fusion proteins having amino acid sequences with certain % sequence identities described herein. In aspects, the nucleic acid is messenger RNA. In aspects, the messenger RNA is messenger RNP.

In embodiments, the vector further includes a polynucleotide, wherein the polynucleotide includes: (1) a DNA-targeting sequence that is complementary to a target polynucleotide sequence; and (2) a binding sequence for the nuclease-deficient RNA-guided DNA endonuclease enzyme. In aspects, the vector further includes a polynucleotide, wherein the polynucleotide includes sgRNA. In aspects, the vector further includes a polynucleotide, wherein the polynucleotide includes cr:tracrRNA. Thus, one or more vectors may include all necessary components for preforming epigenome editing.

Cells

The compositions described herein may be incorporated into a cell. Inside the cell, the compositions as described herein, including embodiments and aspects thereof, may perform epigenome editing. Accordingly, in an aspect is provided a cell including a fusion protein as described herein, including embodiments and aspects thereof, a nucleic acid as described herein, including embodiments and aspects thereof, a complex as described herein, including embodiments and aspects thereof, or a vector as described herein, including embodiments and aspects thereof. In aspects is provided a cell including a fusion protein as described herein, including embodiments and aspects thereof. In aspects is provided a cell including a nucleic acid as described herein, including embodiments and aspects thereof. In aspects is provided a cell including a complex as described herein, including embodiments and aspects thereof. In aspects is provided a cell including a vector as described herein, including embodiments and aspects thereof. In aspects, the cell is a eukaryotic cell.

In aspects, the cell is a mammalian cell. In embodiments, the mammalian cell is a HEK293T cell. In embodiments, the mammalian cell is a T cell. In embodiments, the mammalian cell is a hematopoietic stem cell. In embodiments, the mammalian cell is an induced pluripotent stem cell. In embodiments, the mammalian cell is an embryonic stem cell.

Methods

It is contemplated that the methods described herein may be used for epigenome editing, and more particularly epigenome editing resulting in the activation or reactivation of target nucleic acid sequences (e.g., genes). The methods provided herein include recruitment of one or more fusion proteins for multiplex editing of the DNA epigenetic code and the histone code. The methods allow for long-term but reversible activation of transcription, and may be used to activate previously silenced genes. The methods provided herein may be used for therapeutic purposes. For example, recruitment of one or more fusion proteins provided herein may activate gene expression by editing negative regulatory sequences. This method may be used for editing sequences that block expression of genes.

The fusion proteins described herein program a durable memory of gene activation over time. Gene activation (or reactivation) is achieved by transfection of mRNA encoding the fusion proteins described herein. Thus, transient expression of the fusion protein leads to effective gene activation (or reactivation). CRISPRon epigenetic memory using the fusion proteins described herein is propagated by the cell rather than by sustained transgene expression.

In embodiments, the disclosure provides methods of activating a target nucleic acid sequence in a cell, the method comprising: (i) delivering a first polynucleotide encoding a fusion protein, as described herein, including all embodiments and aspects thereof (e.g., comprising a nuclease-deficient RNA-guided DNA endonuclease enzyme), to a cell containing the target nucleic acid; and (ii) delivering to the cell a second polynucleotide comprising: (a) a sgRNA or (b) a cr:tracrRNA; thereby activating the target nucleic acid sequence in the cell. In embodiments, the second polynucleotide comprises the sgRNA. In embodiments, the sgRNA comprises at least one MS2 stem loop. In embodiments, the sgRNA comprises two MS2 stem loops. In aspects, the target nucleic acid sequence comprises a CpG island. In aspects, the target nucleic acid sequence comprises a non-CpG island.

In embodiments, the disclosure provides methods of activating a target nucleic acid sequence in a cell, the method comprising delivering a polynucleotide encoding a fusion protein, as described herein, including all embodiments and aspects thereof (e.g., comprising a nuclease-deficient DNA endonuclease enzyme), to a cell containing the target nucleic acid; thereby activating the target nucleic acid sequence in the cell. In aspects, the target nucleic acid sequence comprises a CpG island. In aspects, the target nucleic acid sequence comprises a non-CpG island.

In embodiments, the disclosure provides methods of reactivating a silenced target nucleic acid sequence in a cell, the method comprising: (i) delivering a first polynucleotide encoding a fusion protein, as described herein, including all embodiments and aspects thereof (e.g., comprising a nuclease-deficient RNA-guided DNA endonuclease enzyme), to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In embodiments, the second polynucleotide comprises the sgRNA. In embodiments, the sgRNA comprises at least one MS2 stem loop. In embodiments, the sgRNA comprises two MS2 stem loops. In aspects, the target nucleic acid sequence comprises a CpG island. In aspects, the target nucleic acid sequence comprises a non-CpG island.

In embodiments, the disclosure provides methods of reactivating a target nucleic acid sequence in a cell, the method comprising delivering a polynucleotide encoding a fusion protein, as described herein, including all embodiments and aspects thereof (e.g., comprising a nuclease-deficient DNA endonuclease enzyme), to a cell containing the target nucleic acid; thereby reactivating the target nucleic acid sequence in the cell. In aspects, the target nucleic acid sequence comprises a CpG island. In aspects, the target nucleic acid sequence comprises a non-CpG island.

In embodiments, the disclosure provides methods of activating a target nucleic acid sequence in a cell, the method comprising: (i) delivering a polynucleotide encoding a fusion protein, as described herein, including all embodiments and aspects thereof (e.g., comprising a nuclease-deficient RNA-guided DNA endonuclease enzyme), to a cell containing the target nucleic acid; wherein the polynucleotide further encodes (a) a sgRNA or (b) a cr:tracrRNA; thereby activating the target nucleic acid sequence in the cell. In embodiments, the polynucleotide comprises the sgRNA. In embodiments, the sgRNA comprises at least one MS2 stem loop. In embodiments, the sgRNA comprises two MS2 stem loops. In aspects, the target nucleic acid sequence comprises a CpG island. In aspects, the target nucleic acid sequence comprises a non-CpG island.

In embodiments, the disclosure provides methods of reactivating a silenced target nucleic acid sequence in a cell, the method comprising: delivering a polynucleotide encoding a fusion protein, as described herein, including all embodiments and aspects thereof (e.g., comprising a nuclease-deficient RNA-guided DNA endonuclease enzyme), to a cell containing the silenced target nucleic acid; wherein the polynucleotide further encodes (a) a sgRNA or (b) a cr:tracrRNA; thereby reactivating the silenced target nucleic acid sequence in the cell. In embodiments, the polynucleotide comprises the sgRNA. In embodiments, the sgRNA comprises at least one MS2 stem loop. In embodiments, the sgRNA comprises two MS2 stem loops. In aspects, the target nucleic acid sequence comprises a CpG island. In aspects, the target nucleic acid sequence comprises a non-CpG island.

In the methods of activing a target nucleic acid sequence or reactivating a silenced target nucleic acid sequence described herein, the target nucleic acid comprises a CpG island and a non-CpG island. “Comprises a CpG island” or “comprises a non-CpG island” refers to one or more CpG islands or non-CpG islands, respectively. In aspects, the target nucleic acid sequence comprises a plurality of CpG islands (e.g., 2, 3, 4, 5, or more CpG islands). In aspects, the target nucleic acid sequence comprises a plurality of non-CpG islands (e.g., 2, 3, 4, 5, or more non-CpG islands). In aspects, the target nucleic acid sequence does not comprise a CpG island and does not comprises a non-CpG island.

In embodiments, the MS2 stem loop comprises the sequence of SEQ ID NO:19. In embodiments, the MS2 stem loop has the sequence of SEQ ID NO:19. In aspects, the MS2 stem loop has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:19. In aspects, the MS2 stem loop has a sequence that has at least 85% sequence identity to SEQ ID NO:19. In aspects, the MS2 stem loop has a sequence that has at least 90% sequence identity to SEQ ID NO:19. In aspects, the MS2 stem loop has a sequence that has at least 95% sequence identity to SEQ ID NO:19. In aspects, the MS2 stem loop has a sequence that has at least 50%, 55%, 60%, 65%, 70%, 75%, 80%, 85%, 90%, 91%, 92%, 93%, 94%, 95%, 96%, 97%, 98%, 99% or 100% sequence identity to SEQ ID NO:20. In aspects, the MS2 stem loop has a sequence that has at least 85% sequence identity to SEQ ID NO:20. In aspects, the MS2 stem loop has a sequence that has at least 90% sequence identity to SEQ ID NO:20. In aspects, the MS2 stem loop has a sequence that has at least 95% sequence identity to SEQ ID NO:20.

In embodiments, the second polynucleotide further encodes a second fusion protein which comprises a transcriptional activator. In embodiments, the transcriptional activator is VP64, p65, Rta, or a combination of two or more thereof. In embodiments, the transcriptional activator is VP64. In embodiments, the transcriptional activator is p65. In embodiments, the transcriptional activator is Rta. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta.

In embodiments, the second fusion protein comprises an MS2 RNA-binding sequence. In embodiments, the MS2 RNA-binding sequence comprises MCP protein or a functional fragment thereof.

In embodiments, the method further comprises delivering to the cell a third polynucleotide encoding a second fusion protein which comprises a transcriptional activator. In embodiments, the transcriptional activator is VP64, p65, Rta, or a combination of two or more thereof. In embodiments, the transcriptional activator is VP64. In embodiments, the transcriptional activator is p65. In embodiments, the transcriptional activator is Rta. In embodiments, the transcriptional activator comprises VP64, p65, Rta, or a combination of two or more thereof. In embodiments, the transcriptional activator comprises VP64. In embodiments, the transcriptional activator comprises p65. In embodiments, the transcriptional activator comprises Rta. In embodiments, the transcriptional activator comprises VP64 and p65. In embodiments, the transcriptional activator comprises VP64 and Rta. In embodiments, the transcriptional activator comprises p65 and Rta. In embodiments, the transcriptional activator comprises VP64, p65, and Rta.

For the methods provided herein, in embodiments the second fusion protein further comprises an XTEN linker, an epitope tag, a 2A peptide, a fluorescent protein tag, a nuclear localization signal peptide, or a combination of two or more thereof. In embodiments, the second fusion protein further comprises an XTEN linker. In embodiments the second fusion protein further comprises an epitope tag. In embodiments the second fusion protein further comprises a 2A peptide. In embodiments the second fusion protein further comprises a fluorescent protein tag. In embodiments the second fusion protein further comprises a nuclear localization signal peptide.

The term “CpG island” is used in its customary sense to refer to regions in an nucleic acid that have a high frequency of the nucleotides G and C next to one another (i.e., CpG dinucleotides). In aspects, a CpG island refers to a region of a nucleic acid sequence having at least 200 base pair, and a GC content greater than 50%, with an observed-to-expected CpG ratio greater than 60%. The percentage CpG is the ratio of CpG nucleotide bases (twice the CpG count) to the length. The ratio of observed to expected CpG is calculated according to the formula:

Obs/Exp CpG=Number of CpG*N/(Number of C*Number of G),

where N=length of sequence. See Gardiner-Garden et al, Journal of Molecular Biology, 196(2):261-282 (1987)).

The phrase “target nucleic acid does not comprise a CpG island” or “target nucleic acid that does not comprise a CpG island” or “non-CpG island” refers to a target nucleic acid that does not contain a “CpG island” as that term is defined herein. This region can be any region encoded by a mammalian (e.g., human) genome. In aspects, the phrase “target nucleic does not comprise a CpG island” refers to regions in a target nucleic acid that have do not have the nucleotides G and C next to one another (i.e., CpG dinucleotides) or that have a low frequency of the nucleotides G and C next to one another. In aspects, a non-CpG island refers to regions of a target nucleic acid having a region with a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 50%, with an observed-to-expected CpG ratio less than 60%. In aspects, a non-CpG island refers to regions of a target nucleic acid having a GC dinucleotide content less than 45%, with an observed-to-expected CpG ratio less than 55%. In aspects, a non-CpG island refers to regions of a target nucleic acid having a GC dinucleotide content less than 40%, with an observed-to-expected CpG ratio less than 50%. In aspects, a non-CpG island refers to regions of a target nucleic acid having a GC dinucleotide content of 1% to 45%, with an observed-to-expected CpG ratio of less than 60%. In aspects, a non-CpG island refers to regions of a target nucleic acid having a GC dinucleotide content of 1% to 45%, with an observed-to-expected CpG ratio less than 55%. In aspects, a non-CpG island refers to regions of a target nucleic acid a GC dinucleotide content of 1% to 45%, with an observed-to-expected CpG ratio less than 50%. In aspects, a non-CpG island refers to regions of a target nucleic acid having a GC dinucleotide content of 5% to 40%, with an observed-to-expected CpG ratio less than 60%. In aspects, a non-CpG island refers to regions of a target nucleic acid having a GC dinucleotide content of 5% to 40%, with an observed-to-expected CpG ratio less than 55%. In aspects, a non-CpG island refers to regions of a target nucleic acid having a GC dinucleotide content of 5% to 40%, with an observed-to-expected CpG ratio less than 50%. In aspects, a non-CpG island refers to regions of a target nucleic acid having a GC dinucleotide content of 10% to 40%, with an observed-to-expected CpG ratio less than 60%. In aspects, a non-CpG island refers to regions of a target nucleic acid having a GC dinucleotide content of 10% to 40%, with an observed-to-expected CpG ratio less than 55%. In aspects, a non-CpG island refers to regions of a target nucleic acid having a GC dinucleotide content of 10% to 40%, with an observed-to-expected CpG ratio less than 50%. In aspects, the target nucleic acid that does not comprise a CpG island has less than 200 base pairs.

Embodiments 1-69.

Embodiment 1. A fusion protein comprising from N-terminus to C-terminus, a demethylation domain, an XTEN linker, and a nuclease-deficient RNA-guided DNA endonuclease enzyme.

Embodiment 2. The fusion protein of Embodiment 1, wherein the demethylation domain is a TET1 domain, a TET2 domain, a TET3 domain, or a combination of two or more thereof.

Embodiment 3. The fusion protein of Embodiment 2, wherein the demethylation domain is a TET1 domain.

Embodiment 4. The fusion protein of Embodiment 2, wherein the TET1 domain comprises an amino acid sequence having at least 90% sequence identity to SEQ ID NO:1, SEQ ID NO:86, or SEQ ID NO:97.

Embodiment 5. The fusion protein of any one of Embodiments 1 to 4, wherein the nuclease-deficient RNA-guided DNA endonuclease enzyme is dCas9, dCas12a, dCpf1, Cas-phi, a helix-turn-helix motif, a helix-loop-helix domain, an HMB-box domain, a Wor3 domain, an OB-fold domain, an immunoglobulin domain, or a B3 domain.

Embodiment 6. The fusion protein of Embodiment 5, wherein the nuclease-deficient RNA-guided DNA endonuclease enzyme is dCas9.

Embodiment 7. The fusion protein of any one of Embodiments 1 to 6, wherein the XTEN linker comprises from about 10 amino acid residues to about 864 amino acid residues.

Embodiment 8. The fusion protein of Embodiment 7, wherein the XTEN linker comprises an amino acid sequence having at least 90% sequence identity to SEQ ID NO:5, SEQ ID NO:6 or SEQ ID NO:98.

Embodiment 9. The fusion protein of any one of Embodiments 1 to 8, wherein the fusion protein further comprises an epitope tag, a 2A peptide, a fluorescent protein tag, a nuclear localization signal peptide, or a combination of two or more thereof.

Embodiment 10. A fusion protein comprising from N-terminus to C-terminus, an RNA-binding sequence, an XTEN linker, and at least one transcriptional activator.

Embodiment 11. The fusion protein of Embodiment 10, wherein the transcriptional activator is VP64, p65, Rta, or a combination of two or more thereof.

Embodiment 12. The fusion protein of Embodiment 11, wherein p65 comprises an amino acid sequence having at least 90% sequence identity to SEQ ID NO:13, SEQ ID NO:14, or SEQ ID NO:100.

Embodiment 13. The fusion protein of Embodiment 11 or 12, wherein Rta comprises an amino acid sequence having at least 90% sequence identity to SEQ ID NO:15 or SEQ ID NO:16.

Embodiment 14. The fusion protein of any one of Embodiments 11 to 13, wherein VP64 comprises an amino acid sequence having at least 90% sequence identity to SEQ ID NO:17 or SEQ ID NO:18.

Embodiment 15. The fusion protein of any one of Embodiments 10 to 14, wherein the RNA-binding sequence is an MS2 RNA-binding sequence.

Embodiment 16. The fusion protein of Embodiment 15, wherein the MS2 RNA-binding sequence comprises the amino acid sequence of SEQ ID NO:21.

Embodiment 17. The fusion protein of any one of Embodiments 10 to 16, wherein the XTEN linker comprises from about 10 amino acid residues to about 864 amino acid residues.

Embodiment 18. The fusion protein of Embodiment 10 having an amino acid sequence with at least 90% sequence identity to SEQ ID NO:104, SEQ ID NO:105, SEQ ID NO:106, SEQ ID NO:107, SEQ ID NO:108, SEQ ID NO:109, or SEQ ID NO:110.

Embodiment 19. The fusion protein of any one of Embodiments 10 to 18, wherein the fusion protein further comprises an epitope tag, a 2A peptide, a fluorescent protein tag, a nuclear localization signal peptide, or a combination of two or more thereof.

Embodiment 20. A fusion protein comprising from N-terminus to C-terminus, a demethylation domain, a first XTEN linker, a nuclease-deficient RNA-guided DNA endonuclease enzyme, a second XTEN linker, and a transcriptional activator.

Embodiment 21. The fusion protein of Embodiment 20, wherein the transcriptional activator is VP64, p65, Rta, or a combination of two or more thereof.

Embodiment 22. A fusion protein comprising from N-terminus to C-terminus, a demethylation domain, an XTEN linker, and a nuclease-deficient RNA-guided DNA endonuclease enzyme.

Embodiment 23. The fusion protein of any one of Embodiments 20 to 22, further comprising a nuclear localization sequence.

Embodiment 24. The fusion protein of any one of Embodiments 20 to 23, wherein the demethylation domain is a TET1 domain, a TET2 domain, a TET3 domain, or a combination of two or more thereof.

Embodiment 25. The fusion protein of Embodiment 24, wherein the demethylation domain is a TET1 domain.

Embodiment 26. The fusion protein of any one of Embodiments 20 to 25, wherein the nuclease-deficient RNA-guided DNA endonuclease enzyme is dCas9, dCas12a, dCpf1, Cas-phi, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

Embodiment 27. The fusion protein of Embodiment 26, wherein the nuclease-deficient RNA-guided DNA endonuclease enzyme is dCas9.

Embodiment 28. The fusion protein of any one of Embodiments 20 to 27, wherein the first XTEN linker and the second XTEN linker each independently comprise from about 10 amino acid residues to about 864 amino acid residues.

Embodiment 29. The fusion protein of any one of Embodiments 20 to 28, wherein the fusion protein further comprising an epitope tag, a 2A peptide, a fluorescent protein tag, or a combination of two or more thereof.

Embodiment 30. A fusion protein comprising an amino acid sequence having at least 90% sequence identity to SEQ ID NO:99, SEQ ID NO:101, SEQ ID NO:102, SEQ ID NO:111, SEQ ID NO:112, or SEQ ID NO:113.

Embodiment 31. The fusion protein of Embodiment 30, comprising an amino acid sequence having at least 95% sequence identity to SEQ ID NO:99, SEQ ID NO:101, SEQ ID NO:102, SEQ ID NO:111, SEQ ID NO:112, or SEQ ID NO:113.

Embodiment 32. The fusion protein of Embodiment 31 comprising SEQ ID NO:99, SEQ ID NO:101, SEQ ID NO:102, SEQ ID NO:111, SEQ ID NO:112, or SEQ ID NO:113.

Embodiment 33. A method of activating or reactivating a target nucleic acid sequence in a cell, the method comprising: (i) delivering a first polynucleotide encoding a fusion protein of any one of Embodiments 1 to 32 to a cell containing the target nucleic acid; and (ii) delivering to the cell a second polynucleotide comprising: (a) a sgRNA or (b) a cr:tracrRNA; thereby activating or reactivating the target nucleic acid sequence in the cell.

Embodiment 34. The method of Embodiment 32, wherein the target nucleic acid sequence comprises a CpG island.

Embodiment 35. The method of Embodiment 32, wherein the target nucleic acid sequence comprises a non-CpG island.

Embodiment 36. The method of any one of Embodiments 32 to 35, wherein the second polynucleotide comprises the sgRNA.

Embodiment 37. The method of any one of Embodiments 32 to 36, wherein the sgRNA comprises at least one MS2 stem loop.

Embodiment 38. The method of Embodiment 37, wherein the sgRNA comprises two MS2 stem loops.

Embodiment 39. The method of any one of Embodiments 32 to 38, wherein the second polynucleotide encodes a transcriptional activator.

Embodiment 40. The method of Embodiment 39, wherein the transcriptional activator is VP64, p65, Rta, or a combination of two or more thereof.

Embodiment 41. The method of any one of Embodiments 32 to 40, wherein the second polynucleotide further encodes an MS2 RNA-binding sequence.

Embodiment 42. The method of Embodiment 41, wherein the MS2 RNA-binding sequence comprises the amino acid sequence of SEQ ID NO:21.

Embodiment 43. The method of any one of Embodiments 32 to 42, wherein the second polynucleotide further encodes for an XTEN linker, an epitope tag, a 2A peptide, a fluorescent protein tag, a nuclear localization signal peptide, or a combination of two or more thereof.

Embodiment 44. The method of any one of Embodiments 32 to 43, further comprising delivering to the cell a third polynucleotide encoding a second fusion protein which comprises a transcriptional activator.

Embodiment 45. The method of Embodiment 44, wherein the transcriptional activator is VP64, p65, Rta, or a combination of two or more thereof.

Embodiment 46. The method of Embodiment 44 or 45, wherein the second fusion protein further comprises an MS2 RNA-binding sequence.

Embodiment 47. The method of Embodiment 46, wherein the MS2 RNA-binding sequence comprises the amino acid sequence of SEQ ID NO:21.

Embodiment 48. The method of any one of Embodiments 44 to 47, wherein the second fusion protein further comprises an XTEN linker, an epitope tag, a 2A peptide, a fluorescent protein tag, a nuclear localization signal peptide, or a combination of two or more thereof.

Embodiment 49. A fusion protein comprising from N-terminus to C-terminus, a demethylation domain, an XTEN linker, and a nuclease-deficient DNA endonuclease enzyme.

Embodiment 50. The fusion protein of Embodiment 49, wherein the demethylation domain is a TET1 domain, a TET2 domain, a TET3 domain, or a combination of two or more thereof.

Embodiment 51. The fusion protein of Embodiment 49, wherein the demethylation domain is a TET1 domain.

Embodiment 52. The fusion protein of Embodiment 51, wherein the TET1 domain comprises an amino acid sequence having at least 90% sequence identity to SEQ ID NO:1, SEQ ID NO:86, or SEQ ID NO:97.

Embodiment 53. The fusion protein of any one of Embodiments 49 to 52, wherein the nuclease-deficient DNA endonuclease enzyme is a zinc finger domain.

Embodiment 54. The fusion protein of any one of Embodiments 49 to 52, wherein the nuclease-deficient DNA endonuclease enzyme is a TALE.

Embodiment 55. The fusion protein of any one of Embodiments 49 to 54, wherein the XTEN linker comprises from about 10 amino acid residues to about 864 amino acid residues.

Embodiment 56. The fusion protein of Embodiment 55, wherein the XTEN linker comprises an amino acid sequence having at least 90% sequence identity to SEQ ID NO:5, SEQ ID NO:6 or SEQ ID NO:98.

Embodiment 57. The fusion protein of any one of Embodiments 49 to 56 wherein the fusion protein further comprises an epitope tag, a 2A peptide, a fluorescent protein tag, a nuclear localization signal peptide, or a combination of two or more thereof.

Embodiment 58. A fusion protein comprising from N-terminus to C-terminus, a demethylation domain, a first XTEN linker, a nuclease-deficient DNA endonuclease enzyme, a second XTEN linker, and a transcriptional activator.

Embodiment 59. The fusion protein of Embodiment 58, wherein the transcriptional activator is VP64, p65, Rta, or a combination of two or more thereof.

Embodiment 60. A fusion protein comprising from N-terminus to C-terminus, a demethylation domain, an XTEN linker, and a nuclease-deficient DNA endonuclease enzyme.

Embodiment 61. The fusion protein of any one of Embodiments 58 to 60, further comprising a nuclear localization sequence.

Embodiment 62. The fusion protein of any one of Embodiments 58 to 61, wherein the demethylation domain is a TET1 domain, a TET2 domain, a TET3 domain, or a combination of two or more thereof.

Embodiment 63. The fusion protein of Embodiment 62, wherein the demethylation domain is a TET1 domain.

Embodiment 64. The fusion protein of any one of Embodiments 58 to 63, wherein the nuclease-deficient DNA endonuclease enzyme is a zinc finger domain.

Embodiment 65. The fusion protein of any one of Embodiments 58 to 63, wherein the nuclease-deficient DNA endonuclease enzyme is a TALE.

Embodiment 66. The fusion protein of any one of Embodiments 58 to 65, wherein the first XTEN linker and the second XTEN linker each independently comprise from about 10 amino acid residues to about 864 amino acid residues.

Embodiment 67. The fusion protein of any one of Embodiments 58 to 66, wherein the fusion protein further comprising an epitope tag, a 2A peptide, a fluorescent protein tag, or a combination of two or more thereof.

Embodiment 68. A method of activating or reactivating a target nucleic acid sequence in a cell, the method comprising delivering a polynucleotide encoding a fusion protein of any one of Embodiments 58 to 67 to a cell containing the target nucleic acid; thereby activating or reactivating the target nucleic acid sequence in the cell.

Embodiment 69. The method of Embodiment 68, wherein the transcriptional activator is VP64, p65, Rta, or a combination of two or more thereof.

### EXAMPLES

Embodiments and aspects herein are further illustrated by the following examples. The examples are merely intended to illustrate embodiments and aspects, and are not to be construed to limit the scope herein.

### Example 1

Gene Silencing is Reversible by Targeted DNA Methylation

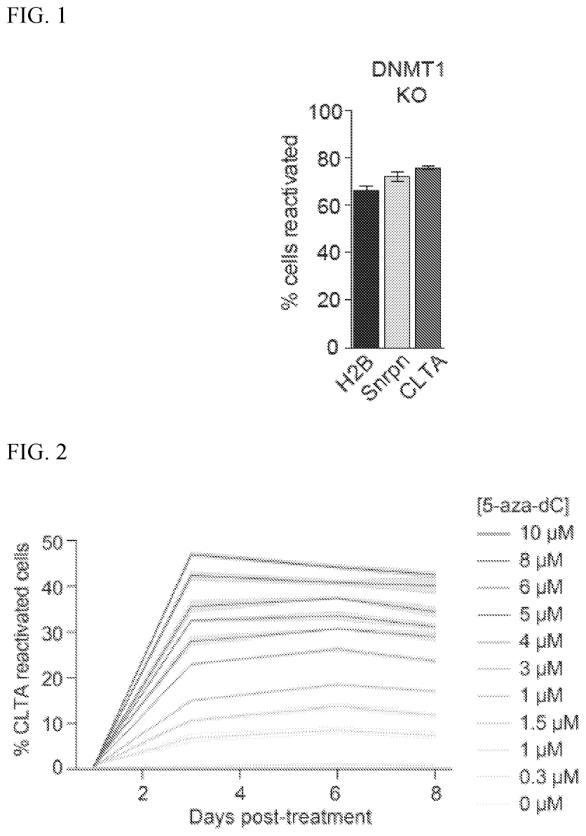

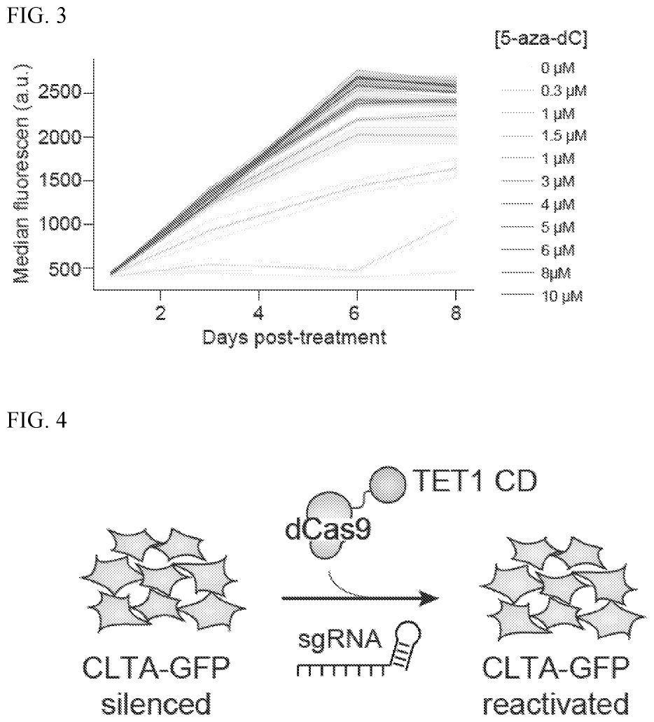

An attractive property of epigenome editing is the ability to reverse the epigenetic changes induced by artificial editors. To test the reversibility of CRISPRoff-mediated gene silencing, we first utilized global methods to block DNA methylation maintenance during cell division. We used Cas9 gene editing to inactivate DNMT1—the main DNA methylation maintenance enzyme in mammalian cells—in HEK293T cells with previously silenced H2B, CLTA, or Snrpn-GFP. At 9 days post knock-out of DNMT1, 60-80% of cells reactivate gene expression. Loss of DNMT1, which is an essential gene, has a noticeable cytotoxic effect and precludes DNMT1 knockout as a feasible method of reactivating CRISPRoff-silenced genes (FIG. 1). Similarly, treatment of cells with a small molecule inhibitor of DNMT1, 5-aza-2′-deoxycytidine (5-aza-dC), reactivated CLTA gene expression albeit at lower efficiency compared to a DNMT1 knockout (FIGS. 2-3). These results demonstrate depletion of DNA methylation is sufficient to reverse CRISPRoff gene silencing. Therefore, we sought to engineer gene-specific and programmable tools for reactivation of CRISPRoff-silenced genes.

### Example 2

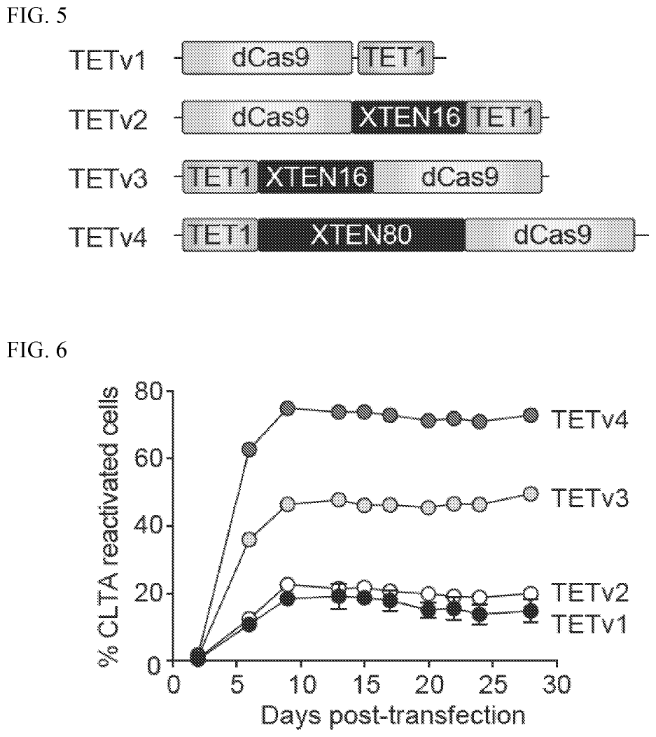

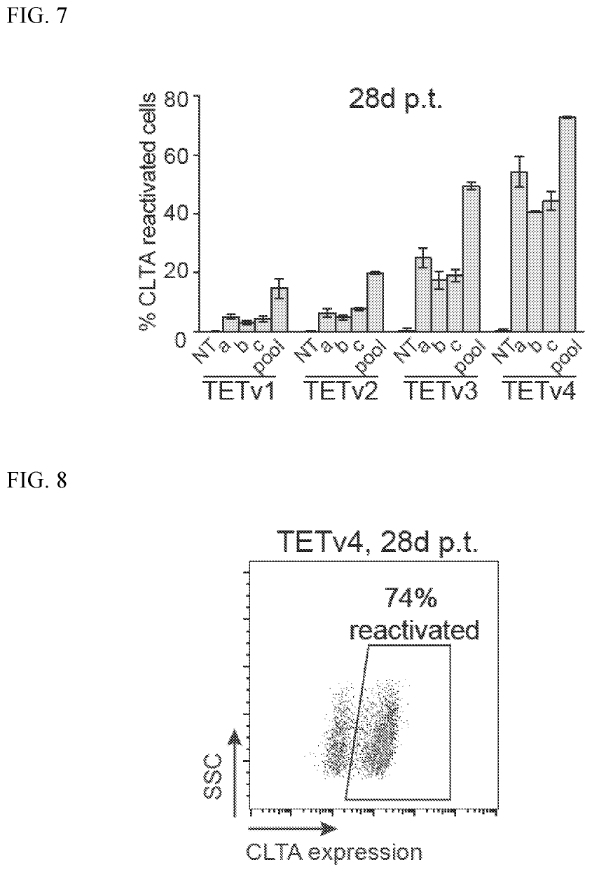

DNA methylation of cytosines within a cytosine-guanine dyad can be actively removed by the TET (ten-eleven translocation) family enzymes, which have been repurposed for programmable demethylation of human gene promoters for gene activation. We tested whether we could reactivate CRISPRoff-silenced genes by targeted DNA demethylation of CLTA, a gene that we silenced for more than 1 year. Initially, we used a previously reported dCas9 fusion to the TET1 DNA demethylase catalytic domain (TETv1) (Liu et al, Cell, 167-233-247 (2016)). We co-transfected plasmids expressing TETv1 and sgRNAs targeting the CLTA promoter, and measured CLTA protein levels (GFP) over time. (FIGS. 4-5). Our results demonstrate targeted DNA demethylation by TETv1 reactivated gene expression, however at 28 days post-transfection, only about 20% of the transfected cells maintain CLTA expression consistent with the variable reactivation typical in previous studies. (FIG. 6) To improve reactivation, we optimized the fusion proteins by encoding XTEN linkers between dCas9 and TET1, and repositioned TET1 at the N-terminus of dCas9. Placing TET1 at the N-terminus with a 16 amino acid XTEN16 linker (TETv3) improved CLTA reactivation to about 50% of cells. Moreover, separating TET1 and dCas9 with an 80 amino acid XTEN80 linker (TETv4) resulted in stable CLTA reactivation in more than 70% of cells. CLTA reactivation was stable for at least 28 days post-transfection (FIGS. 6-8). Gene reactivation was achieved in up to 60% of TETv4-transfected cells with one sgRNA sequence, but was improved by pooling three sgRNAs across a gene promoter (FIG. 7).

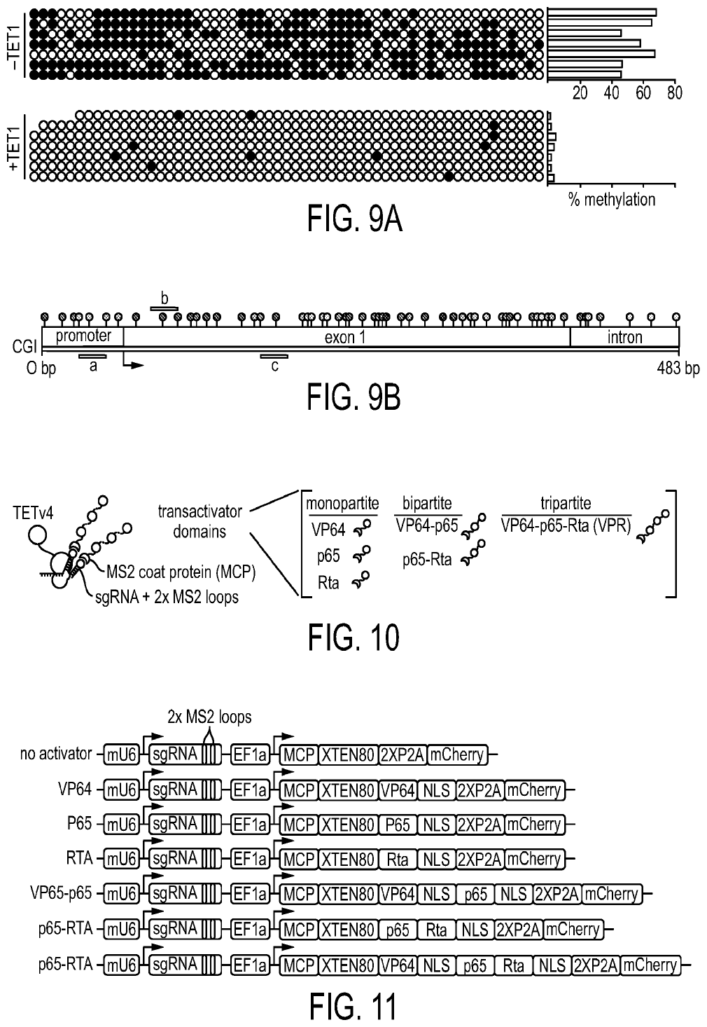

To assess the extent of DNA demethylation across the silenced gene, we performed bisulfite sequencing of the CLTA locus before and after dCas9-TET-mediated reactivation. We observed high levels of DNA methylation along the entire CLTA CGI after CRISPRoff-mediated silencing, including >400 bp downstream of the sgRNA binding sites. (FIGS. 9A-9B) After TET1-mediated gene reactivation, the CGI is demethylated to near-completion, correlating with full reactivation of CLTA expression (FIG. 9A).

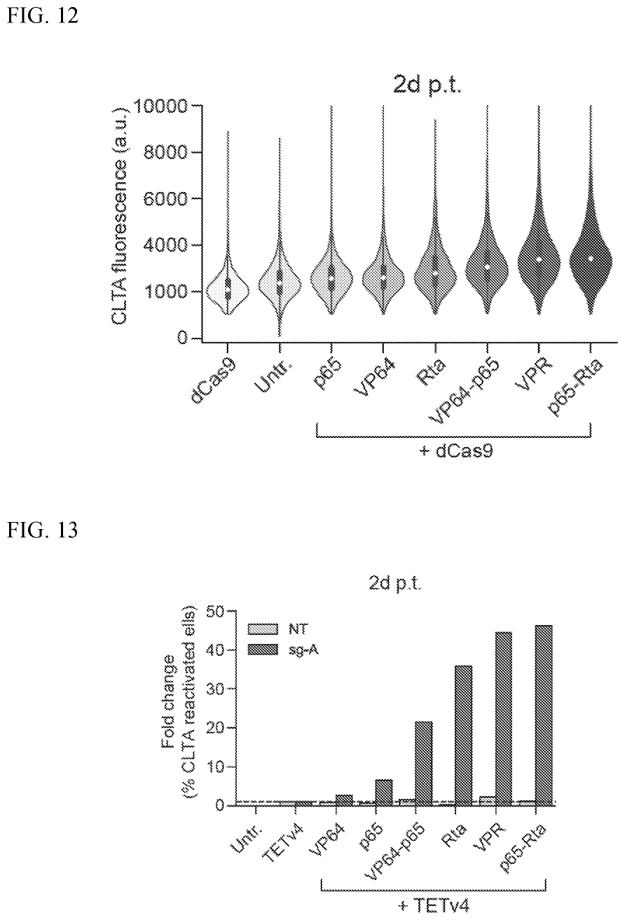

We observed that CLTA reactivation consistently peaks and stabilizes starting at 9 days post-TET1 treatment. (FIG. 6). We hypothesized that gene expression could be reactivated at earlier time points by recruiting transcriptional activator domains to TET1v4. In an effort to modulate the kinetics of gene reactivation, we designed a system called CRISPRon, composed of TETv4, a previously reported modified sgRNA that encodes two MS2 stem sequences, and MS2 coat protein (MCP) fused to various combinations of transcriptional transactivator domains VP64, p65 (p65-AD), and Rta (Konermann et al., 2015a) (FIGS. 10-11). We first confirmed that co-expression of dCas9 and MCP-transactivator fusion proteins could increase gene expression in the absence of TET1, we fused the domains to the MS2 coat protein (MCP) and recruited the fusions to dCas9 targeted to the promoter of endogenously expressed CLTA with sgRNAs encoding MS2 loops. Two days post transfection of dCas9, the MCP fusions, and sgRNAs, we detect increased endogenous expression of the CLTA gene with each transactivator combination with highest reactivation using VPR and p65-Rta (FIG. 12), indicating that these proteins are functional for recruiting the transcriptional machinery.

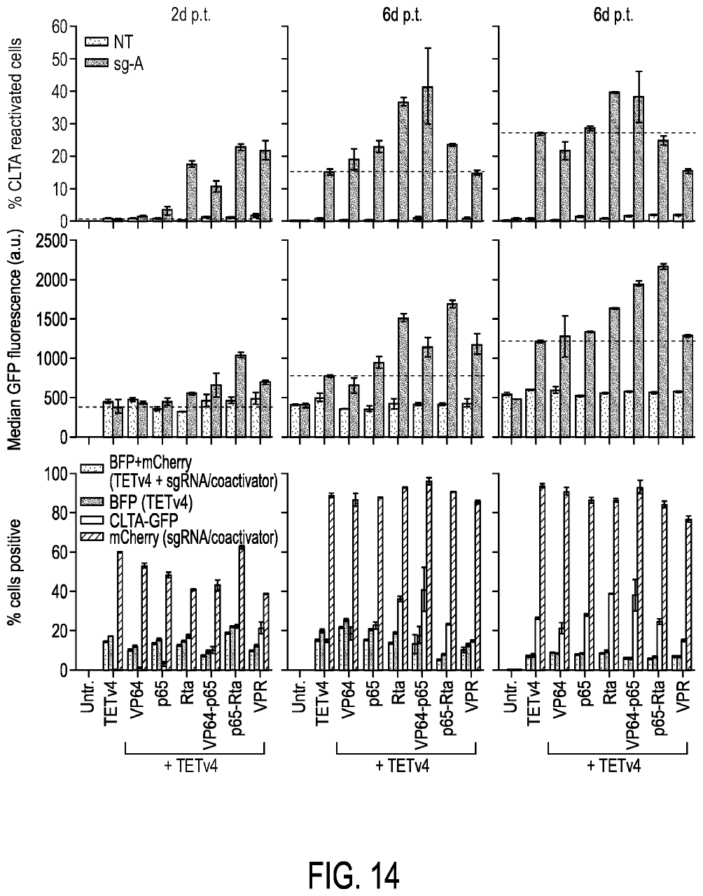

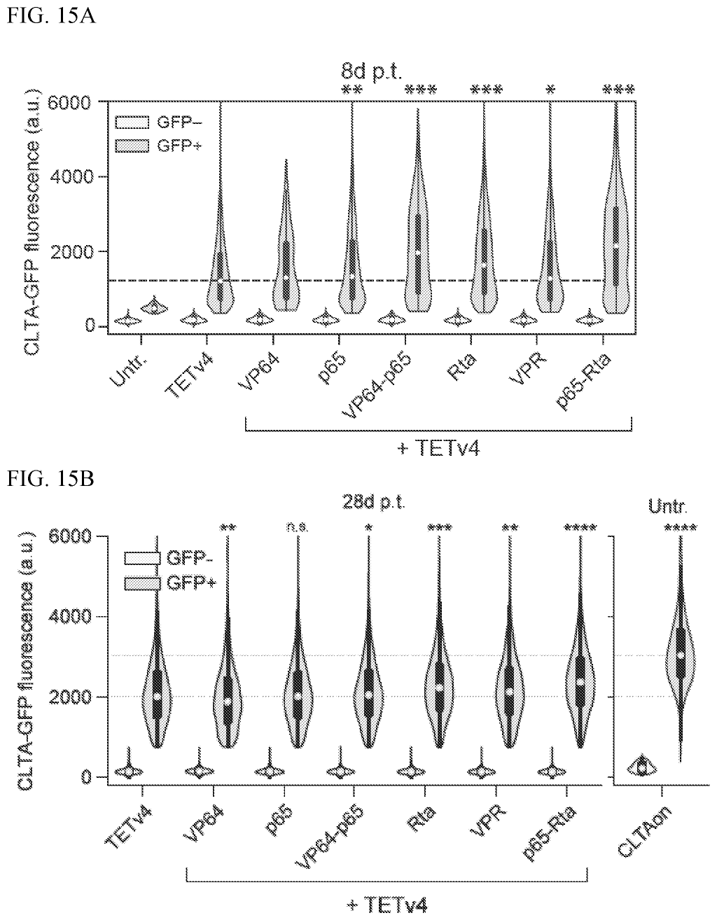

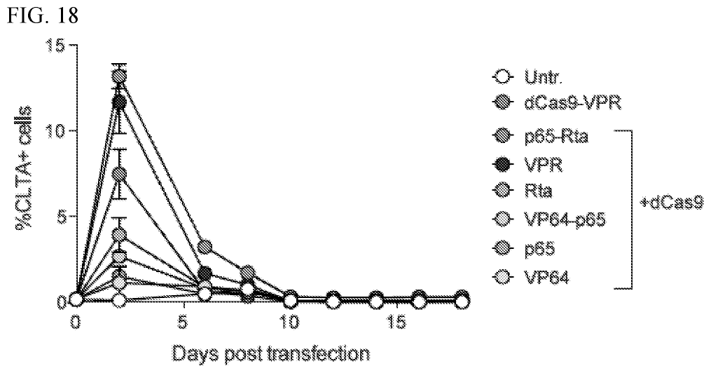

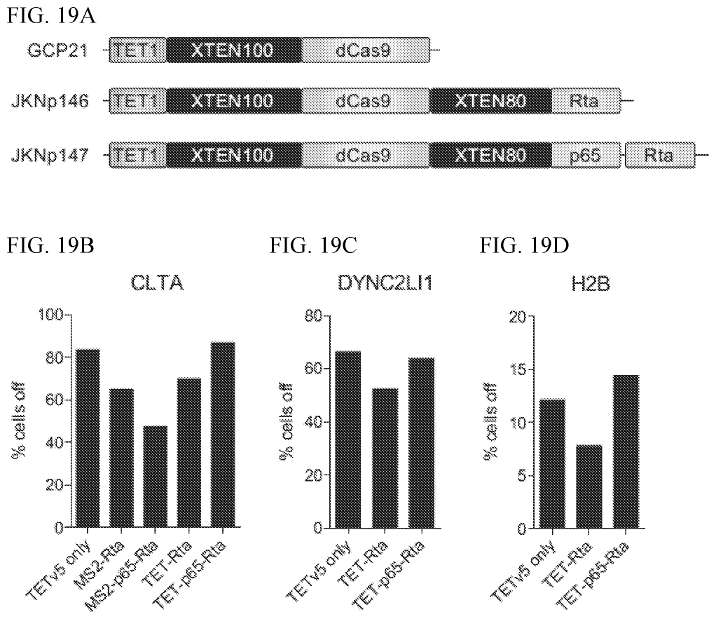

We then expressed negative control (NT) or CLTA-targeting sgRNAs (sg-A) along with various CRISPRon combinations or TETv4 only in CLTA silenced cells and monitored CLTA expression over time. Unexpectedly, we observed that select CRISPRon combinations, such as TETv4 with p65-Rta and TETv4 with VPR, robustly reactivated CLTA expression within 2 days. Meanwhile, TETv4 showed little gene reactivation at this time point (FIGS. 13 and 17). We next co-recruited the transactivators and TETv4 to the CRISPRoff-silenced CLTA promoter. Two days post transfection, CLTA expression reactivates only in the presence of TETv4 and transactivators (FIGS. 13 and 14). Each transactivator combination increases the fraction of cells with CLTA reactivated at varying levels, ranging from 2- to 46-fold compared to TETv4 only, with VPR and p65-Rta eliciting the highest levels of CLTA expression. By eight days post transfection, recruitment of monopartite Rta or VP64-p65 results in the highest increase in the fraction of reactivated cells compared to the other transactivators (FIGS. 14 and 15A). TETv4 and sgRNA-coactivator are present at low levels in cells at this time point (<10% of cells), signifying that increased expression of the reactivated gene using TETv4 with p65-Rta or VP64-p65 is inheritable and memorized by cells. By 28 days post-transfection, the median fluorescence of reactivated CLTA-GFP was significantly higher with CRISPRon combinations of TETv4 with Rta and TETv4 with p65-Rta compared to TETv4 only (FIG. 15B). We do not detect TETv4 or MCP fusion protein expression at this time point. As a further control, co-expressing the MCP transactivator fusions with dCas9 (no TET), or a single fusion dCas9-VPR, showed only transient activation of CLTA and by 10 days post-transfection, CLTA levels revert to the silenced state (FIG. 18). Together, these results show that our optimized TET1-dCas9 fusion proteins can robustly reactivate CRISPRoff-silenced genes as a form of transcriptional memory and the dynamics of reactivation can be further modulated using our CRISPRon combinations. Taken together, these data highlight our ability to modulate the dynamics of reactivation of CRISPRoff-silenced genes and encode cellular memory of gene expression, akin to hit-and-run CRISPRa.

### Example 3

Silencing and Re-Activating Genes that Lack a CpG Annotation

To validate our observation that CRISPRoff can turn off genes without annotated CGIs, we endogenously tagged five genes with no annotated CGI in HEK293T with mNeonGreen (mNG) and assessed durable silencing by CRISPRoff. At 9 days post-transfection, we detect high percentage of cells that have turned off DYNC2LI1, LAMP2, MYL6, and VPS25. Silencing of DYNC2LI1 and LAMP2 remains stable through 14 days post-transfection and MYL6 and VPS25 displayed cell growth defects upon knockdown. Transfection of the CRISPRoff Dnmt3A mutant does not sustain gene silencing and thus, the observed durable phenotype is DNA methylation-dependent. In contrast, CRISPRoff transfection into CALD1-mNG cells did not result in silencing with CRISPRoff or CRISPRoff mutant, suggesting that the gene is not amenable to DNA methylation-dependent hit-and-run epigenome editing.

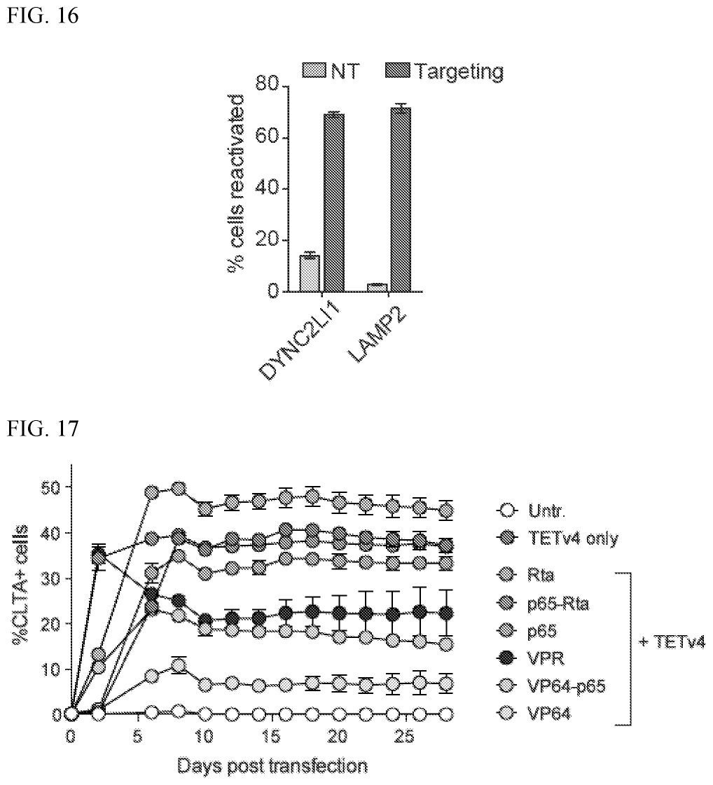

We isolated cells that have turned off LAMP2, DYNC2LI1, and MYL6 by CRISPRoff and profiled the DNA methylation status of the promoters by bisulfite sequencing. Analysis of cytosines within a CG context were highly methylated in silenced cells. Moreover, we treated DYNC2LI1 and LAMP2-off cells with TETv4 and about 70% of cells reactivate the silenced gene by 14 days post transfection of TETv4 (FIG. 16).

Materials and Methods for Examples

Plasmid Design and Construction

The TETv1 design was constructed by PCR amplification of the dCas9-TET1CD sequence from Fuw-dCas9-Tet1CD (Addgene #84475) and assembled into a CAG-expression plasmid. XTEN linker sequences were previously published (Schellenberger et al). All CRISPRoff and TET1 fusion proteins include BFP as either a direct fusion or with a P2A-cleavage sequence to measure transfection efficiency by flow cytometry. The dSaCas9 (D10A, N508A) sequence was PCR amplified from pX603 (Addgene #61594) and the dLbCas12a sequence was PCR amplified from Tak et al. VP64, p65, and Rta were PCR amplified from SP-dCas9-VPR (Addgene #63798). The GAPDH-Snrpn-GFP lentiviral reporter originated from Addgene #70148 (Liu et al., 2016; Stelzer et al., 2015).

The sgRNA plasmids were constructed by restriction cloning of protospacers downstream of a U6 promoter using BstXI and BlpI cut sites, as previously described. The sgRNA expression plasmids also express a T2A-mCherry marker to measure transfection efficiency. The sgRNA sequences used for CRISPRoff and CRISPRon experiments are listed in Table 1. The sgRNA sequences were chosen based on our previous algorithm to predict active CRISPRi sgRNAs (Horlbeck et al., 2016).

The MS2 plasmids were constructed by first transferring the mU6 promoter-sgRNA-EF1a-puromycin-T2A-mCherry cassette into a non-lentiviral vector by restriction cloning. The MCP-XTEN80-NLS-(transactivator domain)-2×P2A cassette was ordered as four gBlocks (IDT) and cloned into the aforementioned non-lentiviral plasmid by Gibson assembly. The sgRNA-MS2 loops sequence was designed based on the SAM system (Konermann et al., 2015b) with the BstXI and BlpI restriction sites incorporated from our previous mU6 sgRNA expression design (Addgene #84832). The DNA sequence encoding the MS2-sgRNA scaffold is SEQ ID NO:117. For construction of the transactivator plasmids, each domain or combination of domains was PCR amplified and cloned by Gibson assembly into a plasmid that encodes the sgRNA and MS2 coat protein (MCP). Guide sequences were cloned by double digest and ligation of annealed oligos, as previously described.

All mRNA constructs were synthesized using the mMESSAGE mMachine™ T7 Ultra Transcription Kit (Thermo Fisher Scientific). The T7 promoter sequence (SEQ ID NO:118) was first cloned upstream of the CRISPRoff sequence. The T7-CRISPRoff sequence was PCR amplified and used as template for in vitro synthesis reactions. Following the manufacturer protocol for synthesis, the reactions were cleaned by chloroform extraction and isopropanol precipitation.

Cell Culture, DNA Transfections, and Flow Cytometry

All cell lines were cultured at 37° C. with 5% CO2 tissue culture incubators. HEK293T (female), HeLa (female), and U2OS (female) cells were cultured in Dulbecco's modified eagle medium (DMEM) in 10% FBS (HyClone), 100 units/mL streptomycin, 100 μg/ml penicillin, and 2 mM glutamine. K562 (female) cells were maintained in RPMI-1640 with 25 mM HEPES and 2.0 g/L NaHCo3 in 10% FBS, 2 mM glutamine, 100 units/mL streptomycin, and 100 mg/mL penicillin. WTC Gen1c iPSCs (male) were cultured in mTESR media (STEMCELL Technologies) under feeder-free conditions on growth factor-reduced Matrigel (BD Biosciences). Cells were passaged using Accutase (STEMCELL Technologies) and seeded on Matrigel coated plates with mTESR media supplemented with p16-Rho-associated coiled-coil kinase (ROCK) inhibitor Y-27632 (10 μM; Selleckchem).

Lentiviral particles were produced by transfecting standard packaging vectors into HEK293T using TransIT-LT1 Transfection Reagent (Minis, MIR2306). Media was changed 24 hours post-transfection with complete DMEM supplemented with 15 mM HEPES. Viral supernatants were harvested 48-60 hours after transfection and filtered through a 0.45 μm PVDF syringe filter. Lentiviral infections included polybrene (8 μg/ml).

CRISPRon

All CRISPRon experiments were conducted in 24-well plates. Briefly, 1×10⁵ CLTA-GFP-silenced HEK293T cells were seeded in each well. When cells reached 60-80% confluency the next day, cells were transfected with 500 ng of dCas9 plasmid (dCas9 or TETv1-4) and 300 ng of sgRNA-transactivator plasmid (sgRNA only, VP64, p65, Rta, VP64-p65, p65-Rta, or VPR). Cells were monitored for BFP (dCas9 or TETv1-4) and mCherry (guide-transactivator) expression 24 hours after transfection. Two days post-transfection, 7.5×10⁴ BFP and mCherry double positive cells were sorted using a BD FACSAria Fusion sorter. Cells were allowed to recover for 4 days after the sort and were subsequently analyzed every 2-3 days using flow cytometry on a Attune NxT cytometer (Thermo Fisher Scientific). All flow cytometry data were analyzed using Flowjo.

RNA Sequencing

HEK293T cells that have maintained stable silencing of target genes were harvested 33 days (ITGB1, CD81, and CD151) or 28 days (CLTA, HIST2H2BE, RAB11A, and VIM) post CRISPRoff transfection. Cells were dislodged from plates with PBS, centrifuged at 500×g for 5 min and washed again with PBS. Total RNA was extracted using Direct-zol RNA MiniPrep (Zymo R2051). Library preparations were carried out using TruSeq Stranded mRNA Library Preparation Kit (Illumina RS-111-2101), starting with 1000 ng total RNA. Final libraries were assessed using a 2100 Bioanalyzer (Agilent), quantified using Qubit dsDNA HS Assay Kit (Thermo Fisher Scientific), and sequenced as single end 50 base pair reads on a HiSeq 4000 (Illumina). For processing the sequencing reads, linker sequences (SEQ ID NO:119) were removed using FASTX-clipper (FASTX-Toolkit). The reads were then aligned to the human genome (GRCh37) using the STAR (Spliced Transcripts Alignment to a Reference, version 2.5) aligner against the Gencode Gene V24lift37 transcriptome annotation. Read quantification was carried out with featureCounts (Liao et al., 2014). All downstream analyses were performed with Python (version 2.7) using a combination of Numpy (v1.12.1), Pandas (v0.17.1), and Scipy (v0.17.0) libraries. Knockdown efficiency was calculated by normalizing gene Transcripts per Million (TPM) for the experimental samples with the mean TPM of the control (non-targeting) samples. Differential expression analysis was performed using DESeq2 (Love et al., 2014).

Quantitative PCR

For quantitative PCR (qPCR) measurements, total RNA was first extracted using RNeasy Micro Kit (Qiagen) from cells. Reverse transcription of 1 μg total RNA was performed using Superscript™ III Reverse Transcriptase kit (Thermo Fisher Scientific) supplemented with RNaseOut™ Recombinant Ribonuclease Inhibitor (Thermo Fisher Scientific). Reverse transcription was primed using oligo(dT)20. Quantitative PCR reactions were prepared with KAPA SYBR FAST qPCR Master Mix (2×) and run on a LightCycler 480 Instrument (Roche). Primer sequences for qPCR experiments are listed in Table 2.

Bisulfite Sequencing PCR

For methylation analysis of the CLTA CGI, 2×10⁶ CRISPRoff-silenced cells and TET-reactivated cells were isolated by FACS. Genomic DNA was extracted from cells according to manufacturer's instructions using the PureLink Genomic DNA Mini Kit (Invitrogen). For each condition, 1 ug genomic DNA underwent bisulfite conversion and cleanup according to manufacturer's instructions using the EpiTect Bisulfite kit (Qiagen). Purified bisulfite-converted DNA was amplified using EpiMark Hot Start Taq (NEB) with a nested PCR method (Liu et al., 2016). Amplicons were gel purified using a Gel DNA Recovery Kit (Zymo) and PCR amplified again using EpiMark Hot Start Taq. Amplicons were cloned into the pCR2.1 TOPO vector according to manufacturer's instructions using the TOPO TA Cloning Kit (Invitrogen). Cloning products were transformed into Stellar E. coli cells (Takara) and plated on blue-white carbenicillin plates. 20 colonies were picked per condition and sequenced by Sanger sequencing. Primer sequences for bisulfite-PCR amplification are listed in Table 2. The primer sequences for amplifying the GAPDH-Snrpn fragment was obtained from Liu et al.

Cas9 Genome Editing and 5-Aza-dC Treatments

Lentiviral particles expressing Cas9 from S. pyogenes were transduced into HEK293T cells that have CRISPRoff-silenced Snrpn-GFP or GFP-tagged CLTA and H2B. Cas9-expressing cells, marked by BFP fluorescence in the lentivirus vector, were FACS-sorted. To inactive DNMT1, lentiviral particles expressing a sgRNA that targets DNMT1 were infected into the cell lines. Reactivation of the silenced genes was assessed by GFP activation, measured by flow cytometry. The last time point was taken at 9 days post sgRNA infection, as cell viability was severely reduced past this time point.

For 5-aza-dC treatment, 1×10⁵ CRISPRoff-silenced CLTA-GFP HEK293T cells were seeded in each well of a 24-well plate. 24 hours later, the media was aspirated and replaced with media supplemented aqueous 5-Aza-2′-deoxycytidine (5-aza-dC) for a final volume of 500 ml per well. The following day, 5-aza-dC-containing media was aspirated, cells were detached and analyzed for viability and GFP activation on a Attune NxT flow cytometer (Thermo Fisher Scientific). Cells were subsequently passaged with fresh media every 2-3 days and analyzed on a Attune cytometer.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### REFERENCES

Adamson, et al. (2016). A Multiplexed Single-Cell CRISPR Screening Platform Enables Systematic Dissection of the Unfolded Protein Response. Cell 167, 1867-1882.e21. Alanis-Lobato, et al (2020). Frequent loss-of-heterozygosity in CRISPR-Cas9-edited early human embryos. BioRxiv 2020.06.05.135913. Amabile, et al (2016). Inheritable Silencing of Endogenous Genes by Hit-and-Run Targeted Epigenetic Editing. Cell 167, 219-232.e14. Anzalone, et al (2020). Genome editing with CRISPR-Cas nucleases, base editors, transposases and prime editors. Nat. Biotechnol. 38, 824-844. Blomen, et al (2015). Gene essentiality and synthetic lethality in haploid human cells. Science 350, 1092-1096. Bothmer, et al. (2020). Detection and Modulation of DNA Translocations During Multi-Gene Genome Editing in T Cells. CRISPR J. Boyes, J., and Bird, A. (1992). Repression of genes by DNA methylation depends on CpG density and promoter strength: evidence for involvement of a methyl-CpG binding protein. EMBO J. 11, 327-333. Cheng, et al (2013). Multiplexed activation of endogenous genes by CRISPR-on, an RNA-guided transcriptional activator system. Cell Res. 23, 1163-1171. Choudhury, et al (2016). CRISPR-dCas9 mediated TET1 targeting for selective DNA demethylation at BRCA1 promoter. Oncotarget 7, 46545-46556. Deaton, A. M., and Bird, A. (2011). CpG islands and the regulation of transcription. Genes Dev. 25, 1010-1022. Dede, et al (2020). Multiplex enCas12a screens show functional buffering by paralogs is systematically absent from genome-wide CRISPR/Cas9 knockout screens. BioRxiv 2020.05.18.102764. Doench, J. G. (2018). Am I ready for CRISPR? A user's guide to genetic screens. Nat. Rev. Genet. 19, 67-80. El-Brolosy, M. A., and Stainier, D. Y. R. (2017). Genetic compensation: A phenomenon in search of mechanisms. PLoS Genet. 13. ENCODE Project Consortium, Moore, J. E., et al. (2020). Expanded encyclopaedias of DNA elements in the human and mouse genomes. Nature 583, 699-710. Ferrari, S., et al (2011). Retinitis Pigmentosa: Genes and Disease Mechanisms. Curr. Genomics 12, 238-249. Fulco, C. P., et al (2016). Systematic mapping of functional enhancer-promoter connections with CRISPR interference. Science 354, 769-773. Gilbert, et al. (2013). CRISPR-Mediated Modular RNA-Guided Regulation of Transcription in Eukaryotes. Cell 154, 442-451. Gilbert, L. A., et al. (2014). Genome-Scale CRISPR-Mediated Control of Gene Repression and Activation. Cell 159, 647-661. Gong, G., et al (2004). Genetic dissection of myocilin glaucoma. Hum. Mol. Genet. 13 Spec No 1, R91-102. Halmai, et al. (2020). Artificial escape from XCI by DNA methylation editing of the CDKLS gene. Nucleic Acids Res. 48, 2372-2387. Hanna, R. E., and Doench, J. G. (2020). Design and analysis of CRISPR-Cas experiments. Nat. Biotechnol. 38, 813-823. Hart, T., et al (2014). Measuring error rates in genomic perturbation screens: gold standards for human functional genomics. Mol. Syst. Biol. 10, 733. He, Y., et al. (2020). Spatiotemporal DNA methylome dynamics of the developing mouse fetus. Nature 583, 752-759. Hilton, et al (2015). Epigenome editing by a CRISPR-Cas9-based acetyltransferase activates genes from promoters and enhancers. Nat. Biotechnol. 33, 510-517. Holtzman, L., and Gersbach, C. A. (2018). Editing the Epigenome: Reshaping the Genomic Landscape. Annu. Rev. Genomics Hum. Genet. 19, 43-71. Horlbeck, M. A., et al. (2016). Compact and highly active next-generation libraries for CRISPR-mediated gene repression and activation. ELife 5, e19760. Ihry, R. J., et al. (2018). p53 inhibits CRISPR-Cas9 engineering in human pluripotent stem cells. Nat. Med. 24, 939-946. Jia, D., et al (2007). Structure of Dnmt3a bound to Dnmt3L suggests a model for de novo DNA methylation. Nature 449, 248-251. Josipović, G., et al. (2019). Antagonistic and synergistic epigenetic modulation using orthologous CRISPR/dCas9-based modular system. Nucleic Acids Res. 47, 9637-9657. Jost, M., et al. (2020). Titrating gene expression using libraries of systematically attenuated CRISPR guide RNAs. Nat. Biotechnol. 38, 355-364. Kearns, et al (2014). Cas9 effector-mediated regulation of transcription and differentiation in human pluripotent stem cells. Dev. Camb. Engl. 141, 219-223. Knott, G. J., and Doudna, J. A. (2018). CRISPR-Cas guides the future of genetic engineering. Science 361, 866-869. Konermann, et al (2013). Optical control of mammalian endogenous transcription and epigenetic states. Nature 500, 472-476. Konermann, et al. (2015a). Genome-scale transcriptional activation by an engineered CRISPR-Cas9 complex. Nature 517, 583-588. Konermann, et al. (2015b). Genome-scale transcriptional activation by an engineered CRISPR-Cas9 complex. Nature 517, 583-588. Kosicki, M., Tomberg, K., and Bradley, A. (2018). Repair of double-strand breaks induced by CRISPR-Cas9 leads to large deletions and complex rearrangements. Nat. Biotechnol. 36, 765-771. La Spada, A. R., and Taylor, J. P. (2010). Repeat expansion disease: Progress and puzzles in disease pathogenesis. Nat. Rev. Genet. 11, 247-258. Leonetti, et al (2016a). A scalable strategy for high-throughput GFP tagging of endogenous human proteins. Proc. Natl. Acad. Sci. U.S.A 113, E3501-3508. Leonetti, et al (2016b). A scalable strategy for high-throughput GFP tagging of endogenous human proteins. Proc. Natl. Acad. Sci. 113, E3501-E3508. Li, X.-L., et al. (2018). Highly efficient genome editing via CRISPR-Cas9 in human pluripotent stem cells is achieved by transient BCL-XL overexpression. Nucleic Acids Res. 46, 10195-10215. Liang, D., et al. (2020). Frequent Gene Conversion in Human Embryos Induced by Double Strand Breaks. BioRxiv 2020.06.19.162214. Liao, et al (2014). featureCounts: an efficient general purpose program for assigning sequence reads to genomic features. Bioinformatics 30, 923-930. Liu, et al (2016). Editing DNA Methylation in the Mammalian Genome. Cell 167, 233-247.e17. Liu, et al. (2018). Rescue of Fragile X Syndrome Neurons by DNA Methylation Editing of the FMR1 Gene. Cell 172, 979-992.e6. Love, M. I., Huber, W., and Anders, S. (2014). Moderated estimation of fold change and dispersion for RNA-seq data with DESeq2. Genome Biol. 15, 550. Maeder, et al (2013a). CRISPR RNA-guided activation of endogenous human genes. Nat. Methods 10, 977-979. Maeder, et al. (2013b). Targeted DNA demethylation and activation of endogenous genes using programmable TALE-TET1 fusion proteins. Nat. Biotechnol. 31, 1137-1142. Mali, P., et al (2013). CAS9 transcriptional activators for target specificity screening and paired nickases for cooperative genome engineering. Nat. Biotechnol. 31, 833-838. Meyers, et al. (2017). Computational correction of copy number effect improves specificity of CRISPR-Cas9 essentiality screens in cancer cells. Nat. Genet. 49, 1779-1784. Michlits, et al. (2020). Multilayered VBC score predicts sgRNAs that efficiently generate loss-of-function alleles. Nat. Methods 17, 708-716. Mlambo, et al (2018). Designer epigenome modifiers enable robust and sustained gene silencing in clinically relevant human cells. Nucleic Acids Res. 46, 4456-4468. Morita, et al. (2016). Targeted DNA demethylation in vivo using dCas9-peptide repeat and scFv-TET1 catalytic domain fusions. Nat. Biotechnol. 34, 1060-1065. Morita, et al (2020). Synergistic Upregulation of Target Genes by TET1 and VP64 in the dCas9-SunTag Platform. Int. J. Mol. Sci. 21. O'Geen, et al (2017). dCas9-based epigenome editing suggests acquisition of histone methylation is not sufficient for target gene repression. Nucleic Acids Res. 45, 9901-9916. O'Geen, H., et al (2019). Ezh2-dCas9 and KRAB-dCas9 enable engineering of epigenetic memory in a context-dependent manner. Epigenetics Chromatin 12, 26. Perez-Pinera, P., et al. (2013). RNA-guided gene activation by CRISPR-Cas9-based transcription factors. Nat. Methods 10, 973-976. Replogle, et al. (2020). Combinatorial single-cell CRISPR screens by direct guide RNA capture and targeted sequencing. Nat. Biotechnol. 38, 954-961. Roth, T. L., et al. (2018). Reprogramming human T cell function and specificity with non-viral genome targeting. Nature 559, 405-409. Schellenberger, et al. (2009). A recombinant polypeptide extends the in vivo half-life of peptides and proteins in a tunable manner. Nat. Biotechnol. 27, 1186-1190. Schumann, et al. (2015). Generation of knock-in primary human T cells using Cas9 ribonucleoproteins. Proc. Natl. Acad. Sci. 112, 10437-10442. Shalem, et al (2015). High-throughput functional genomics using CRISPR-Cas9. Nat. Rev. Genet. 16, 299-311. Shifrut, et al (2018). Genome-wide CRISPR Screens in Primary Human T Cells Reveal Key Regulators of Immune Function. Cell 175, 1958-1971.e15. Stelzer, et al (2015). Tracing Dynamic Changes of DNA Methylation at Single-Cell Resolution. Cell 163, 218-229. Tak, et al (2017). Inducible and multiplex gene regulation using CRISPR-Cpf1-based transcription factors. Nat. Methods 14, 1163-1166. Tarjan, et al (2019). Epigenome editing strategies for the functional annotation of CTCF insulators. Nat. Commun. 10, 4258. Tian, et al. (2019). CRISPR Interference-Based Platform for Multimodal Genetic Screens in Human iPSC-Derived Neurons. Neuron 104, 239-255.e12. Veitia, R. A., Caburet, S., and Birchler, J. A. (2018). Mechanisms of Mendelian dominance. Clin. Genet. 93, 419-428. Wang, et al (2015). Identification and characterization of essential genes in the human genome. Science 350, 1096-1101. Xu, X., and Qi, L. S. (2019). A CRISPR-dCas Toolbox for Genetic Engineering and Synthetic Biology. J. Mol. Biol. 431, 34-47. Zhang, et al. (2018). Structural basis for DNMT3A-mediated de novo DNA methylation. Nature 554, 387-391. Zuccaro, et al. (2020). Reading frame restoration at the EYS locus, and allele-specific chromosome removal after Cas9 cleavage in human embryos. BioRxiv 2020.06.17.149237.

Informal Sequence Listing

In the sequences listed herein, the skilled artisan will appreciate that a methionine (M) can be present on the N-terminus of protein in order to initiate translation. Thus, the sequences described herein can optionally further comprise a methionone on the N-terminus.

SEQ ID NO: 1 = TET1 (UniProt: Q8NFU7)
MSRSRHARPSRLVRKEDVNKKKKNSQLRKTTKGANKNVASVKTLSPGKLKQLIQERDVKKKTEP
KPPVPVRSLLTRAGAARMNLDRTEVLFONPESLTCNGFTMALRSTSLSRRLSQPPLVVAKSKKV
PLSKGLEKQHDCDYKILPALGVKHSENDSVPMQDTQVLPDIETLIGVQNPSLLKGKSQETTQEW
SQRVEDSKINIPTHSGPAAEILPGPLEGTRCGEGLFSEETLNDTSGSPKMFAQDTVCAPFPQRA
TPKVTSQGNPSIQLEELGSRVESLKLSDSYLDPIKSEHDCYPTSSLNKVIPDLNLRNCLALGGS
TSPTSVIKFLLAGSKQATLGAKPDHQEAFEATANQQEVSDTTSFLGQAFGAIPHQWELPGADPV
HGEALGETPDLPEIPGAIPVQGEVFGTILDQQETLGMSGSVVPDLPVFLPVPPNPIATFNAPSK
WPEPQSTVSYGLAVQGAIQILPLGSGHTPQSSSNSEKNSLPPVMAISNVENEKQVHISFLPANT
QGFPLAPERGLFHASLGIAQLSQAGPSKSDRGSSQVSVTSTVHVVNTTVVTMPVPMVSTSSSSY
TTLLPTLEKKKRKRCGVCEPCQQKTNCGECTYCKNRKNSHQICKKRKCEELKKKPSVVVPLEVI
KENKRPQREKKPKVLKADFDNKPVNGPKSESMDYSRCGHGEEQKLELNPHTVENVTKNEDSMTG
IEVEKWTQNKKSQLTDHVKGDFSANVPEAEKSKNSEVDKKRTKSPKLFVQTVRNGIKHVHCLPA
ETNVSFKKFNIEEFGKTLENNSYKFLKDTANHKNAMSSVATDMSCDHLKGRSNVLVFQQPGFNC
SSIPHSSHSIINHHASIHNEGDQPKTPENIPSKEPKDGSPVQPSLLSLMKDRRLTLEQVVAIEA
LTQLSEAPSENSSPSKSEKDEESEQRTASLLNSCKAILYTVRKDLQDPNLQGEPPKLNHCPSLE
KQSSCNTVVFNGQTTTLSNSHINSATNQASTKSHEYSKVTNSLSLFIPKSNSSKIDTNKSIAQG
IITLDNCSNDLHQLPPRNNEVEYCNQLLDSSKKLDSDDLSCQDATHTQIEEDVATQLTQLASII
KINYIKPEDKKVESTPTSLVTCNVQQKYNQEKGTIQQKPPSSVHNNHGSSLTKQKNPTQKKTKS
TPSRDRRKKKPTVVSYQENDRQKWEKLSYMYGTICDIWIASKFQNFGQFCPHDFPTVFGKISSS
TKIWKPLAQTRSIMQPKTVFPPLTQIKLQRYPESAEEKVKVEPLDSLSLFHLKTESNGKAFTDK
AYNSQVQLTVNANQKAHPLTQPSSPPNQCANVMAGDDQIRFQQVVKEQLMHQRLPTLPGISHET
PLPESALTLRNVNVVCSGGITVVSTKSEEEVCSSSFGTSEFSTVDSAQKNFNDYAMNFFTNPTK
NLVSITKDSELPTCSCLDRVIQKDKGPYYTHLGAGPSVAAVREIMENRYGQKGNAIRIEIVVYT
GKEGKSSHGCPIAKWVLRRSSDEEKVLCLVRQRTGHHCPTAVMVVLIMVWDGIPLPMADRLYTE
LTENLKSYNGHPTDRRCTLNENRTCTCQGIDPETCGASFSFGCSWSMYFNGCKFGRSPSPRRFR
IDPSSPLHEKNLEDNLQSLATRLAPIYKQYAPVAYQNQVEYENVARECRLGSKEGRPFSGVTAC
LDFCAHPHRDIHNMNNGSTVVCTLTREDNRSLGVIPQDEQLHVLPLYKLSDTDEFGSKEGMEAK
IKSGAIEVLAPRRKKRTCFTQPVPRSGKKRAAMMTEVLAHKIRAVEKKPIPRIKRKNNSTTTNN
SKPSSLPTLGSNTETVQPEVKSETEPHFILKSSDNTKTYSLMPSAPHPVKEASPGFSWSPKTAS
ATPAPLKNDATASCGFSERSSTPHCTMPSGRLSGANAAAADGPGISQLGEVAPLPTLSAPVMEP
LINSEPSTGVTEPLTPHQPNHQPSFLTSPQDLASSPMEEDEQHSEADEPPSDEPLSDDPLSPAE
EKLPHIDEYWSDSEHIFLDANIGGVAIAPAHGSVLIECARRELHATTPVEHPNRNHPTRLSLVF
YQHKNLNKPQHGFELNKIKFEAKEAKNKKMKASEQKDQAANEGPEQSSEVNE
LNQIPSHKALTLTHDNVVTVSPYALTHVAGPYNHWV
SEQ ID NO: 2 = TET2 (UniProt Q6N021)
YGIPCMKGSQNSRVSPDFTQESRGYSKCLQNGGIKRTVSEPSLSGLLQIKKLKQDQKANGERRN
FGVSQERNPGESSQPNVSDLSDKKESVSSVAQENAVKDFTSFSTHNCSGPENPELQILNEQEGK
SANYHDKNIVLLKNKAVLMPNGATVSASSVEHTHGELLEKTLSQYYPDCVSIAVQKTTSHINAI
NSQATNELSCEITHPSHTSGQINSAQTSNSELPPKPAAVVSEACDADDADNASKLAAMLNTCSF
QKPEQLQQQKSVFEICPSPAENNIQGTTKLASGEEFCSGSSSNLQAPGGSSERYLKQNEMNGAY
FKQSSVFTKDSFSATTTPPPPSQLLLSPPPPLPQVPQLPSEGKSTLNGGVLEEHHHYPNQSNTT
LLREVKIEGKPEAPPSQSPNPSTHVCSPSPMLSERPQNNCVNRNDIQTAGTMTVPLCSEKTRPM
SEHLKHNPPIFGSSGELQDNCQQLMRNKEQEILKGRDKEQTRDLVPPTQHYLKPGWIELKAPRF
HQAESHLKRNEASLPSILQYQPNLSNQMTSKQYTGNSNMPGGLPRQAYTQKTTQLEHKSQMYQV
EMNQGQSQGTVDQHLQFQKPSHQVHFSKTDHLPKAHVQSLCGTRFHFQQRADSQTEKLMSPVLK
QHLNQQASETEPFSNSHLLQHKPHKQAAQTQPSQSSHLPQNQQQQQKLQIKNKEEILQTFPHPQ
SNNDQQREGSFFGQTKVEECFHGENQYSKSSEFETHNVQMGLEEVQNINRRNSPYSQTMKSSAC
KIQVSCSNNTHLVSENKEQTTHPELFAGNKTQNLHHMQYFPNNVIPKQDLLHRCFQEQEQKSQQ
ASVLQGYKNRNQDMSGQQAAQLAQQRYLIHNHANVFPVPDQGGSHTQTPPQKDTQKHAALRWHL
LQKQEQQQTQQPQTESCHSQMHRPIKVEPGCKPHACMHTAPPENKTWKKVTKQENPPASCDNVQ
QKSIIETMEQHLKQFHAKSLFDHKALTLKSQKQVKVEMSGPVTVLTRQTTAAELDSHTPALEQQ
TTSSEKTPTKRTAASVLNNFIESPSKLLDTPIKNLLDTPVKTQYDFPSCRCVEQIIEKDEGPFY
THLGAGPNVAAIREIMEERFGQKGKAIRIERVIYTGKEGKSSQGCPIAKWVVRRSSSEEKLLCL
VRERAGHTCEAAVIVILILVWEGIPLSLADKLYSELTETLRKYGTLTNRRCALNEERTCACQGL
DPETCGASFSFGCSWSMYYNGCKFARSKIPRKFKLLGDDPKEEEKLESHLQNLSTLMAPTYKKL
APDAYNNQIEYEHRAPECRLGLKEGRPFSGVTACLDFCAHAHRDLHNMQNGSTLVCTLTREDNR
EFGGKPEDEQLHVLPLYKVSDVDEFGSVEAQEEKKRSGAIQVLSSFRRKVRMLAEPVKTCRQRK
LEAKKAAAEKLSSLENSSNKNEKEKSAPSRTKQTENASQAKQLAELLRLSGPVMQQSQQPQPLQ
KQPPQPQQQQRPQQQQPHHPQTESVNSYSASGSTNPYMRRPNPVSPYPNSSHTSDIYGSTSPMN
FYSTSSQAAGSYLNSSNPMNPYPGLLNQNTQYPSYQCNGNLSVDNCSPYLGSYSPQSQPMDLYR
YPSQDPLSKLSLPPIHTLYQPRFGNSQSFTSKYLGYGNQNMQGDGFSSCTIRPNVHHVGKLPPY
PTHEMDGHFMGATSRLPPNLSNPNMDYKNGEHHSPSHIIHNYSAAPGMFNSSLHALHLQNKEND
MLSHTANGLSKMLPALNHDRTACVQGGLHKLSDANGQEKQPLALVQGVASGAEDNDEVWSDSEQ
SFLDPDIGGVAVAPTHGSILIECAKRELHATTPLKNPNRNHPTRISLVFYQHKSMNEPKHGLAL
WEAKMAEKAREKEEECEKYGPDYVPQKSHGKKVKREPAEPHETSEPTYLRFIKSLAERTMSVTT
DSTVTTSPYAFTRVTGPYNRYI
SEQ ID NO: 3 = TET3 (Uniprot O43151)
MSQFQVPLAVQPDLPGLYDFPQRQVMVGSFPGSGLSMAGSESQLRGGGDGRKKRKRCGTCEPCR
RLENCGACTSCTNRRTHQICKLRKCEVLKKKVGLLKEVEIKAGEGAGPWGQGAAVKTGSELSPV
DGPVPGQMDSGPVYHGDSRQLSASGVPVNGAREPAGPSLLGTGGPWRVDQKPDWEAAPGPAHTA
RLEDAHDLVAFSAVAEAVSSYGALSTRLYETFNREMSREAGNNSRGPRPGPEGCSAGSEDLDTL
QTALALARHGMKPPNCNCDGPECPDYLEWLEGKIKSVVMEGGEERPRLPGPLPPGEAGLPAPST
RPLLSSEVPQISPQEGLPLSQSALSIAKEKNISLQTAIAIEALTQLSSALPQPSHSTPQASCPL
PEALSPPAPFRSPQSYLRAPSWPVVPPEEHSSFAPDSSAFPPATPRTEFPEAWGTDTPPATPRS
SWPMPRPSPDPMAELEQLLGSASDYIQSVFKRPEALPTKPKVKVEAPSSSPAPAPSPVLQREAP
TPSSEPDTHQKAQTALQQHLHHKRSLFLEQVHDTSFPAPSEPSAPGWWPPPSSPVPRLPDRPPK
EKKKKLPTPAGGPVGTEKAAPGIKPSVRKPIQIKKSRPREAQPLFPPVRQIVLEGLRSPASQEV
QAHPPAPLPASQGSAVPLPPEPSLALFAPSPSRDSLLPPTQEMRSPSPMTALQPGSTGPLPPAD
DKLEELIRQFEAEFGDSFGLPGPPSVPIQDPENQQTCLPAPESPFATRSPKQIKIESSGAVTVL
STTCFHSEEGGQEATPTKAENPLTPTLSGFLESPLKYLDTPTKSLLDTPAKRAQAEFPTCDCVE
QIVEKDEGPYYTHLGSGPTVASIRELMEERYGEKGKAIRIEKVIYTGKEGKSSRGCPIAKWVIR
RHTLEEKLLCLVRHRAGHHCQNAVIVILILAWEGIPRSLGDTLYQELTDTLRKYGNPTSRRCGL
NDDRTCACQGKDPNTCGASFSFGCSWSMYFNGCKYARSKTPRKFRLAGDNPKEEEVLRKSFQDL
ATEVAPLYKRLAPQAYQNQVTNEEIAIDCRLGLKEGRPFAGVTACMDFCAHAHKDQHNLYNGCT
VVCTLTKEDNRCVGKIPEDEQLHVLPLYKMANTDEFGSEENQNAKVGSGAIQVLTAFPREVRRL
PEPAKSCRQRQLEARKAAAEKKKIQKEKLSTPEKIKQEALELAGITSDPGLSLKGGLSQQGLKP
SLKVEPQNHFSSFKYSGNAVVESYSVLGNCRPSDPYSMNSVYSYHSYYAQPSLTSVNGFHSKYA
LPSFSYYGFPSSNPVFPSQFLGPGAWGHSGSSGSFEKKPDLHALHNSLSPAYGGAEFAELPSQA
VPTDAHHPTPHHQQPAYPGPKEYLLPKAPLLHSVSRDPSPFAQSSNCYNRSIKQEPVDPLTQAE
PVPRDAGKMGKTPLSEVSQNGGPSHLWGQYSGGPSMSPKRTNGVGGSWGVFSSGESPAIVPDKL
SSFGASCLAPSHFTDGQWGLFPGEGQQAASHSGGRLRGKPWSPCKFGNSTSALAGPSLTEKPWA
LGAGDFNSALKGSPGFQDKLWNPMKGEEGRIPAAGASQLDRAWQSFGLPLGSSEKLFGALKSEE
KLWDPFSLEEGPAEEPPSKGAVKEEKGGGGAEEEEEELWSDSEHNFLDENIGGVAVAPAHGSIL
IECARRELHATTPLKKPNRCHPTRISLVFYQHKNLNQPNHGLALWEAKMKQLAERARARQEEAA
RLGLGQQEAKLYGKKRKWGGTVVAEPQQKEKKGVVPTRQALAVPTDSAVTVSSYAYTKVTGPYS
RWI
SEQ ID NO: 4 (SV40 NLS)
PKKKRKV
SEQ ID NO: 5 (XTEN16 (16 amino acid sequence))
SGSETPGTSESATPES
SEQ ID NO: 6 (XTEN80 (80 amino acid sequence))
GGPSSGAPPPSGGSPAGSPTSTEEGTSESATPESGPGTSTEPSEGSAPGSPAGSPTSTEEGTST
EPSEGSAPGTSTEPSE
SEQ ID NO: 7 (HA tag)
YPYDVPDYA
SEQ ID NO: 8 (BFP)
SELIKENMHMKLYMEGTVDNHHFKCTSEGEGKPYEGTQTMRIKVVEGGPLPFAFDILATSFLYG
SKTFINHTQGIPDFFKQSFPEGFTWERVTTYEDGGVLTATQDTSLQDGCLIYNVKIRGVNFTSN
GPVMQKKTLGWEAFTETLYPADGGLEGRNDMALKLVGGSHLIANIKTTYRSKKPAKNLKMPGVY
YVDYRLERIKEANNETYVEQHEVAVARYCDLPSKLGHKLN*
SEQ ID NO: 9 (dCas9)
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGD
SEQ ID NO: 10 (ddAsCfp1)
MTQFEGFTNLYQVSKTLRFELIPQGKTLKHIQEQGFIEEDKARNDHYKELKPIIDRIYKTYADQ
CLQLVQLDWENLSAAIDSYRKEKTEETRNALIEEQATYRNAIHDYFIGRTDNLTDAINKRHAEI
YKGLFKAELFNGKVLKQLGTVTTTEHENALLRSFDKFTTYFSGFYENRKNVFSAEDISTAIPHR
IVQDNFPKFKENCHIFTRLITAVPSLREHFENVKKAIGIFVSTSIEEVFSFPFYNQLLTQTQID
LYNQLLGGISREAGTEKIKGLNEVLNLAIQKNDETAHIIASLPHRFIPLFKQILSDRNTLSFIL
EEFKSDEEVIQSFCKYKTLLRNENVLETAEALFNELNSIDLTHIFISHKKLETISSALCDHWDT
LRNALYERRISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIKLEMEPSLS
EYNKARNYATKKPYSVEKFKLNFQMPTLASGWDVNKEKNNGAILEVKNGLYYLGIMPKQKGRYK
ALSFEPTEKTSEGFDKMYYDYFPDAAKMIPKCSTQLKAVTAHFQTHTTPILLSNNFIEPLEITK
EIYDLNNPEKEPKKFQTAYAKKTGDQKGYREALCKWIDFTRDFLSKYTKTTSIDLSSLRPSSQY
KDLGEYYAELNPLLYHISFQRIAEKEIMDAVETGKLYLFQIYNKDFAKGHHGKPNLHTLYWTGL
FSPENLAKTSIKLNGQAELFYRPKSRMKRMAHRLGEKMLNKKLKDQKTPIPDTLYQELYDYVNH
RLSHDLSDEARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERVAARQAWSV
VGTIKDLKQGYLSQVIHEIVDLMIHYQAVVVLANLNFGFKSKRTGIAEKAVYQQFEKMLIDKLN
CLVLKDYPAEKVGGVLNPYQLTDQFTSFAKMGTQSGFLFYVPAPYTSKIDPLTGFVDPFVWKTI
KNHESRKHFLEGFDFLHYDVKTGDFILHFKMNRNLSFQRGLPGFMPAWDIVFEKNETQFDAKGT
PFIAGKRIVPVIENHRFTGRYRDLYPANELIALLEEKGIVFRDGSNILPKLLENDDSHAIDTMV
ALIRSVLQMRNSNAATGEDYINSPVRDLNGVCFDSRFQNPEWPMDADANGAYHIALKGQLLLNH
LKESKDLKLQNGISNQDWLAYIQELRN
SEQ ID NO: 11 (ddLbCfp1)
MSKLEKFTNCYSLSKTLRFKAIPVGKTQENIDNKRLLVEDEKRAEDYKGVKKLLDRYYLSFIND
VLHSIKLKNLNNYISLFRKKTRTEKENKELENLEINLRKEIAKAFKGNEGYKSLFKKDIIETIL
PEFLDDKDEIALVNSFNGFTTAFTGFFDNRENMFSEEAKSTSIAFRCINENLTRYISNMDIFEK
VDAIFDKHEVQEIKEKILNSDYDVEDFFEGEFFNFVLTQEGIDVYNAIIGGFVTESGEKIKGLN
EYINLYNQKTKQKLPKFKPLYKQVLSDRESLSFYGEGYTSDEEVLEVFRNTLNKNSEIFSSIKK
LEKLFKNFDEYSSAGIFVKNGPAISTISKDIFGEWNVIRDKWNAEYDDIHLKKKAVVTEKYEDD
RRKSFKKIGSFSLEQLQEYADADLSVVEKLKEIIIQKVDEIYKVYGSSEKLFDADFVLEKSLKK
NDAVVAIMKDLLDSVKSFENYIKAFFGEGKETNRDESFYGDFVLAYDILLKVDHIYDAIRNYVT
QKPYSKDKFKLYFQNPQFMGGWDKDKETDYRATILRYGSKYYLAIMDKKYAKCLQKIDKDDVNG
NYEKINYKLLPGPNKMLPKVFFSKKWMAYYNPSEDIQKIYKNGTFKKGDMFNLNDCHKLIDFFK
DSISRYPKWSNAYDFNFSETEKYKDIAGFYREVEEQGYKVSFESASKKEVDKLVEEGKLYMFQI
YNKDFSDKSHGTPNLHTMYFKLLFDENNHGQIRLSGGAELFMRRASLKKEELVVHPANSPIANK
NPDNPKKTTTLSYDVYKDKRFSEDQYELHIPIAINKCPKNIFKINTEVRVLLKHDDNPYVIGIA
RGERNLLYIVVVDGKGNIVEQYSLNEIINNFNGIRIKTDYHSLLDKKEKERFEARQNWTSIENI
KELKAGYISQVVHKICELVEKYDAVIALADLNSGFKNSRVKVEKQVYQKFEKMLIDKLNYMVDK
KSNPCATGGALKGYQITNKFESFKSMSTQNGFIFYIPAWLTSKIDPSTGFVNLLKTKYTSIADS
KKFISSFDRIMYVPEEDLFEFALDYKNFSRTDADYIKKWKLYSYGNRIRIFRNPKKNNVFDWEE
VCLTSAYKELFNKYGINYQQGDIRALLCEQSDKAFYSSFMALMSLMLQMRNSITGRTDVDFLIS
PVKNSDGIFYDSRNYEAQENAILPKNADANGAYNIARKVLWAIGQFKKAEDEKLDKVKIAISNK
EWLEYAQTSVKH
SEQ ID NO: 12 (ddFnCfp1)
MYPYDVPDYASGSGMSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKSAKDTIKKQISEYIK
DSEKFKNLFNQNLIDAKKGQESDLILWLKQSKDNGIELFKANSDITDIDEALEIIKSFKGWTTY
FKGFHENRKNVYSSNDIPTSIIYRIVDDNLPKFLENKAKYESLKDKAPEAINYEQIKKDLAEEL
TFDIDYKTSEVNQRVFSLDEVFEIANFNNYLNQSGITKFNTIIGGKFVNGENTKRKGINEYINL
YSQQINDKTLKKYKMSVLFKQILSDTESKSFVIDKLEDDSDVVTTMQSFYEQIAAFKTVEEKSI
KETLSLLFDDLKAQKLDLSKIYFKNDKSLTDLSQQVFDDYSVIGTAVLEYITQQIAPKNLDNPS
KKEQELIAKKTEKAKYLSLETIKLALEEFNKHRDIDKQCRFEEILANFAAIPMIFDEIAQNKDN
LAQISIKYQNQGKKDLLQASAEDDVKAIKDLLDQTNNLLHKLKIFHISQSEDKANILDKDEHFY
LVFEECYFELANIVPLYNKIRNYITQKPYSDEKFKLNFENSTLANGWDKNKEPDNTAILFIKDD
KYYLGVMNKKNNKIFDDKAIKENKGEGYKKIVYKLLPGANKMLPKVFFSAKSIKFYNPSEDILR
IRNHSTHTKNGSPQKGYEKFEFNIEDCRKFIDFYKQSISKHPEWKDFGFRFSDTQRYNSIDEFY
REVENQGYKLTFENISESYIDSVVNQGKLYLFQIYNKDFSAYSKGRPNLHTLYWKALFDERNLQ
DVVYKLNGEAELFYRKQSIPKKITHPAKEAIANKNKDNPKKESVFEYDLIKDKRFTEDKFFFHC
PITINFKSSGANKFNDEINLLLKEKANDVHILSIARGERHLAYYTLVDGKGNIIKQDTFNIIGN
DRMKTNYHDKLAAIEKDRDSARKDWKKINNIKEMKEGYLSQVVHEIAKLVIEYNAIVVFEDLNE
GFKRGRFKVEKQVYQKLEKMLIEKLNYLVFKDNEFDKTGGVLRAYQLTAPFETFKKMGKQTGII
YYVPAGFTSKICPVTGFVNQLYPKYESVSKSQEFFSKFDKICYNLDKGYFEFSFDYKNFGDKAA
KGKWTIASFGSRLINFRNSDKNHNWDTREVYPTKELEKLLKDYSIEYGHGECIKAAICGESDKK
FFAKLTSVLNTILQMRNSKTGTELDYLISPVADVNGNFFDSRQAPKNMPQDADANGAYHIGLKG
LMLLGRIKNNQEGKKLNLVIKNEEYFEFVONRNN
SEQ ID NO: 13 (p65; UniProt: Q04206)
MDELFPLIFPAEPAQASGPYVEIIEQPKQRGMRFRYKCEGRSAGSIPGERSTDTTKTHPT
IKINGYTGPGTVRISLVTKDPPHRPHPHELVGKDCRDGFYEAELCPDRCIHSFQNLGIQC
VKKRDLEQAISQRIQTNNNPFQVPIEEQRGDYDLNAVRLCFQVTVRDPSGRPLRLPPVLS
HPIFDNRAPNTAELKICRVNRNSGSCLGGDEIFLLCDKVQKEDIEVYFTGPGWEARGSFS
QADVHRQVAIVFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYPFTSSLSTINY
DEFPTMVFPSGQISQASALAPAPPQVLPQAPAPAPAPAMVSALAQAPAPVPVLAPGPPQAVAPP
APKPTQAGEGTLSEALLQLQFDDEDLGALLGNSTDPAVFTDLASVDNSEFQQLLNQGIPVAPHT
TEPMLMEYPEAITRLVTGAQRPPDPAPAPLGAPGLPNGLLSGDEDFSSIADM DFSALLSQISS
SEQ ID NO: 14 (p65; from Addgene)
PTQAGEGTLSEALLQLQFDDEDLGALLGNSTDPAVFTDLASVDNSEFQQLLNQGIPVAPHTTEP
MLMEYPEAITRLVTGAQRPPDPAPAPLGAPGLPNGLLSGDEDFSSIADMDFSALL
SEQ ID NO: 15 (Rta; from Addgene)
RDSREGMFLPKPEAGSAISDVFEGREVCQPKRIRPFHPPGSPWANRPLPASLAPTPTGPVHEPV
GSLTPAPVPQPLDPAPAVTPEASHLLEDPDEETSQAVKALREMADTVIPQKEEAAICGQMDLSH
PPPRGHLDELTTTLESMTEDLNLDSPLTPELNEILDTFLNDECLLHAMHISTGLSIFDTSLF
SEQ ID NO: 16 (Rta; UniProt P03209)
MRPKKDGLEDFLRLTPEIKKQLGSLVSDYCNVLNKEFTAGSVEITLRSYKICKAFINEAKAHGR
EWGGLMATLNICNFWAILRNNRVRRRAENAGNDACSIACPIVMRYVLDHLIVVTDRFFIQAPSN
RVMIPATIGTAMYKLLKHSRVRAYTYSKVLGVDRAAIMASGKQVVEHLNRMEKEGLLSSKFKAF
CKWVFTYPVLEEMFQTMVSSKTGHLTDDVKDVRALIKTLPRASYSSHAGQRSYVSGVLPACLLS
TKSKAVETPILVSGADRMDEELMGNDGGASHTEARYSESGQFHAFTDELESLPSPTMPLKPGAQ
SADCGDSSSSSSDSGNSDTEQSEREEARAEAPRLRAPKSRRTSRPNRGQTPCPSNAAEPEQPWI
AAVHQESDERPIFPHPSKPTFLPPVKRKKGLRDSREGMFLPKPEAGSAISDVFEGREVCQPKRI
RPFHPPGSPWANRPLPASLAPTPTGPVHEPVGSLTPAPVPQPLDPAPAVTPEASHLLEDPDEET
SQAVKALREMADTVIPQKEEAAICGQMDLSHPPPRGHLDELTTTLESMTEDLNLDSPLTPELNE
ILDTFLNDECLLHAMHISTGLSIFDTSLF
SEQ ID NO: 17 (VP64; from Addgene)
DALDDFDLDMLGSDALDDFDLDMLGSDALDDFDLDMLGSDALDDFDLDML
SEQ ID NO: 18 (Full length Tegument protein VP16; VP64; UniProt P06492)
MDLLVDELFADMNADGASPPPPRPAGGPKNTPAAPPLYATGRLSQAQLMPSPPMPVPPAALFNR
LLDDLGFSAGPALCTMLDTWNEDLFSALPTNADLYRECKFLSTLPSDVVEWGDAYVPERTQIDI
RAHGDVAFPTLPATRDGLGLYYEALSRFFHAELRAREESYRTVLANFCSALYRYLRASVRQLHR
QAHMRGRDRDLGEMLRATIADRYYRETARLARVLFLHLYLFLTREILWAAYAEQMMRPDLFDCL
CCDLESWRQLAGLFQPFMFVNGALTVRGVPIEARRLRELNHIREHLNLPLVRSAATEEPGAPLT
TPPTLHGNQARASGYFMVLIRAKLDSYSSFTTSPSEAVMREHAYSRARTKNNYGSTIEGLLDLP
DDDAPEEAGLAAPRLSFLPAGHTRRLSTAPPTDVSLGDELHLDGEDVAMAHADALDDFDLDMLG
DGDSPGPGFTPHDSAPYGALDMADFEFEQMFTDALGIDEYGG
SEQ ID NO: 19 (MS2 stem loop 1)
AGCCAACATGAGGATCACCCATGTCTGCAGGGC
SEQ ID NO: 20 (MS2 stem loop 2)
GGCCAACATGAGGATCACCCATGTCTGCAGGGCC
SEQ ID NO: 21 (MS2 coat protein (MCP))
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IY
SEQ ID NO: 86 (TET1 catalytic domain (TET1CD))
LPTCSCLDRVIQKDKGPYYTHLGAGPSVAAVREIMENRYGQKGNAIRIEIVVYTGKEGKSSHGC
PIAKWVLRRSSDEEKVLCLVRQRTGHHCPTAVMVVLIMVWDGIPLPMADRLYTELTENLKSYNG
HPTDRRCTLNENRTCTCQGIDPETCGASFSFGCSWSMYFNGCKFGRSPSPRRFRIDPSSPLHEK
NLEDNLQSLATRLAPIYKQYAPVAYQNQVEYENVARECRLGSKEGRPFSGVTACLDFCAHPHRD
IHNMNNGSTVVCTLTREDNRSLGVIPQDEQLHVLPLYKLSDTDEFGSKEGMEAKIKSGAIEVLA
PRRKKRTCFTQPVPRSGKKRAAMMTEVLAHKIRAVEKKPIPRIKRKNNSTTTNNSKPSSLPTLG
SNTETVQPEVKSETEPHFILKSSDNTKTYSLMPSAPHPVKEASPGFSWSPKTASATPAPLKNDA
TASCGFSERSSTPHCTMPSGRLSGANAAAADGPGISQLGEVAPLPTLSAPVMEPLINSEPSTGV
TEPLTPHQPNHQPSFLTSPQDLASSPMEEDEQHSEADEPPSDEPLSDDPLSPAEEKLPHIDEYW
SDSEHIFLDANIGGVAIAPAHGSVLIECARRELHATTPVEHPNRNHPTRLSLVFYQHKNLNKPQ
HGFELNKIKFEAKEAKNKKMKASEQKDQAANEGPEQSSEVNELNQIPSHKALTLTHDNVVTVSP
YALTHVAGPYNHWV
SEQ ID NO: 97 (TET1)
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWV
SEQ ID NO: 98 XTEN100
GGSPAGSPTSTEEGTSESATPESGPGTSTEPSEGSAPGSPAGSPTSTEEGTSTEPSEGSAPGTS
TEPSEGSAPGTSESATPESGPGSEPATSGSETPGSE
SEQ ID NO: 99 Fusion Protein JKNp146
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWVGGSPAGSPTSTEEGTSESATPESGPGTSTEPSEGSAPGSPAGSPTSTE
EGTSTEPSEGSAPGTSTEPSEGSAPGTSESATPESGPGSEPATSGSETPGSEMDKKYSIGLAIG
TNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQ
SFLKDDSIDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLY
ETRIDLSQLGGDGGPSSGAPPPSGGSPAGSPTSTEEGTSESATPESGPGTSTEPSEGSAPGSPA
GSPTSTEEGTSTEPSEGSAPGTSTEPSEGSGPKKKRKVAGSRDSREGMELPKPEAGSAISDVFE
GREVCQPKRIRPFHPPGSPWANRPLPASLAPTPTGPVHEPVGSLTPAPVPQPLDPAPAVTPEAS
HLLEDPDEETSQAVKALREMADTVIPQKEEAAICGQMDLSHPPPRGHLDELTTTLESMTEDLNL
DSPLTPELNEILDTFLNDECLLHAMHISTGLSIFDTSLFASGSGPKKKRKV
SEQ ID NO: 99 comprises the following SEQ ID NOS and spacers:
97-98-9-6-GSG-4-AGS-15-ASGSG-4; where GSG, AGS, and ASGSG are peptide
linkers.
SEQ ID NO: 100 (p65)
SQYLPDTDDRHRIEEKRKRTYETFKSIMKKSPFSGPTDPRPPPRRIAVPSRSSASVPKPAPQPY
PFTSSLSTINYDEFPTMVFPSGQISQASALAPAPPQVLPQAPAPAPAPAMVSALAQAPAPVPVL
APGPPQAVAPPAPKPTQAGEGTLSEALLQLQFDDEDLGALLGNSTDPAVFTDLASVDNSEFQQL
LNQGIPVAPHTTEPMLMEYPEAITRLVTGAQRPPDPAPAPLGAPGLPNGLLSGDEDFSSIADMD
FSALL
SEQ ID NO: 101 Fusion Protein JKNp147
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWVGGSPAGSPTSTEEGTSESATPESGPGTSTEPSEGSAPGSPAGSPTSTE
EGTSTEPSEGSAPGTSTEPSEGSAPGTSESATPESGPGSEPATSGSETPGSEMDKKYSIGLAIG
TNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKVMKQLK
RRRYTGWGRLSRKLINGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQ
SFLKDDSIDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSKKLKSVKELL
GITIMERSSFEKNPIDELEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLY
ETRIDLSQLGGDGGPSSGAPPPSGGSPAGSPTSTEEGTSESATPESGPGTSTEPSEGSAPGSPA
GSPTSTEEGTSTEPSEGSAPGTSTEPSEGSGPKKKRKVAGSSQYLPDTDDRHRIEEKRKRTYET
FKSIMKKSPFSGPTDPRPPPRRIAVPSRSSASVPKPAPQPYPFTSSLSTINYDEFPTMVFPSGQ
ISQASALAPAPPQVLPQAPAPAPAPAMVSALAQAPAPVPVLAPGPPQAVAPPAPKPTQAGEGTL
SEALLQLQFDDEDLGALLGNSTDPAVFTDLASVDNSEFQQLLNQGIPVAPHTTEPMLMEYPEAI
TRLVTGAQRPPDPAPAPLGAPGLPNGLLSGDEDFSSIADMDFSALLGSGSGSRDSREGMFLPKP
EAGSAISDVFEGREVCQPKRIRPFHPPGSPWANRPLPASLAPTPTGPVHEPVGSLTPAPVPQPL
DPAPAVTPEASHLLEDPDEETSQAVKALREMADTVIPQKEEAAICGQMDLSHPPPRGHLDELTT
TLESMTEDLNLDSPLTPELNEILDTFLNDECLLHAMHISTGLSIFDTSLFASGSGPKKKRKV
SEQ ID NO: 101 comprises the following SEQ ID NOS and spacers:
97-98-9-6-GSG-4-AGS-100-GSGSGS-15-ASGSG-4; where GSG, AGS, GSGSGS, and
ASGSG are peptide linkers.
SEQ ID NO: 102 Fusion Protein GCP21
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWVGGSPAGSPTSTEEGTSESATPESGPGTSTEPSEGSAPGSPAGSPTSTE
EGTSTEPSEGSAPGTSTEPSEGSAPGTSESATPESGPGSEPATSGSETPGSEMDKKYSIGLAIG
TNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSNFDLAEDAKLQLSK
DTYDDDLDNLLAQIGDQYADLFLAAKNLSDAILLSDILRVNTEITKAPLSASMIKRYDEHHQDL
TLLKALVRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEYFTVYNEL
TKVKYVTEGMRKPAFLSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLKTYAHLFDDKVMKQLK
RRRYTGWGRLSRKLINGIRDKQSGKTILDELKSDGFANRNFMQLIHDDSLTFKEDIQKAQVSGQ
GDSLHEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDAIVPQ
SFLKDDSIDNKVLTRSDKNRGKSDNVPSEEVVKKMKNYWRQLLNAKLITQRKFDNLTKAERGGL
SELDKAGFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLY
ETRIDLSQLGGDGGGGSPKKKRKVDPKKKRKVDPKKKRKV
SEQ ID NO: 102 comprises the following SEQ ID NOS and spacers:
97-98-9-GGGGS-4-D-4-D-4; where GGGGS, D, and D are peptide linkers.
SEQ ID NO: 103-JKNp84: dCas9-TET1
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNELYLASHYEKLKGSPEDNEQKQLEVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGDGGGGSPKKKRKVDPKKKRKVDPKKKRKVGSLPTCSCLDRV
IQKDKGPYYTHLGAGPSVAAVREIMENRYGQKGNAIRIEIVVYTGKEGKSSHGCPIAKWVLRRS
SDEEKVLCLVRQRTGHHCPTAVMVVLIMVWDGIPLPMADRLYTELTENLKSYNGHPTDRRCTLN
ENRTCTCQGIDPETCGASFSFGCSWSMYFNGCKFGRSPSPRRFRIDPSSPLHEKNLEDNLQSLA
TRLAPIYKQYAPVAYQNQVEYENVARECRLGSKEGRPFSGVTACLDFCAHPHRDIHNMNNGSTV
VCTLTREDNRSLGVIPQDEQLHVLPLYKLSDTDEFGSKEGMEAKIKSGAIEVLAPRRKKRTCFT
QPVPRSGKKRAAMMTEVLAHKIRAVEKKPIPRIKRKNNSTTTNNSKPSSLPTLGSNTETVQPEV
KSETEPHFILKSSDNTKTYSLMPSAPHPVKEASPGFSWSPKTASATPAPLKNDATASCGFSERS
STPHCTMPSGRLSGANAAAADGPGISQLGEVAPLPTLSAPVMEPLINSEPSTGVTEPLTPHQPN
HQPSFLTSPQDLASSPMEEDEQHSEADEPPSDEPLSDDPLSPAEEKLPHIDEYWSDSEHIFLDA
NIGGVAIAPAHGSVLIECARRELHATTPVEHPNRNHPTRLSLVFYQHKNLNKPQHGFELNKIKF
EAKEAKNKKMKASEQKDQAANEGPEQSSEVNELNQIPSHKALTLTHDNVVTVSPYALTHVAGPY
NHWV
SEQ ID NO: 103 comprises the following SEQ ID NOS and spacers:
9-GGGGS-4-D-4-D-4-GS-86; where GGGGS, D, D, and GS are peptide linkers.
SEQ ID NO: 104 = GCPp3: MCP-XTEN80-VP64
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSDALDDFDLDMLGSDALDDFDLDMLGSDALDDFD
LDMLGSDALDDFDLDMLASGSGPKKKRKV
SEQ ID NO: 104 comprises the following SEQ ID NOS and spacers:
21-6-GSG-4-AGS-17-ASGSGPKKKRKV; where GSG, AGS, and ASGSGPKKKRKV are
peptide linkers.
SEQ ID NO: 105 = GCPp4: MCP-XTEN80-VP64-p65
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSDALDDFDLDMLGSDALDDFDLDMLGSDALDDFD
LDMLGSDALDDFDLDMLINSRSSGSPKKKRKVGSQYLPDTDDRHRIEEKRKRTYETFKSIMKKS
PFSGPTDPRPPPRRIAVPSRSSASVPKPAPQPYPFTSSLSTINYDEFPTMVFPSGQISQASALA
PAPPQVLPQAPAPAPAPAMVSALAQAPAPVPVLAPGPPQAVAPPAPKPTQAGEGTLSEALLQLQ
FDDEDLGALLGNSTDPAVFTDLASVDNSEFQQLLNQGIPVAPHTTEPMLMEYPEAITRLVTGAQ
RPPDPAPAPLGAPGLPNGLLSGDEDFSSIADMDFSALLASGSGPKKKRKV
SEQ ID NO: 105 comprises the following SEQ ID NOS and spacers:
21-6-GSG-4-AGS-17-INSRSSGS-4-G-100-ASGSG-4; where GSG, AGS, INSRSSGS,
G, and ASGSG are peptide linkers.
SEQ ID NO: 106 = GCPp5: MCP-XTEN80-VP64-p65p-Rta
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSDALDDFDLDMLGSDALDDFDLDMLGSDALDDFD
LDMLGSDALDDFDLDMLINSRSSGSPKKKRKVGSQYLPDTDDRHRIEEKRKRTYETFKSIMKKS
PFSGPTDPRPPPRRIAVPSRSSASVPKPAPQPYPFTSSLSTINYDEFPTMVFPSGQISQASALA
PAPPQVLPQAPAPAPAPAMVSALAQAPAPVPVLAPGPPQAVAPPAPKPTQAGEGTLSEALLQLQ
FDDEDLGALLGNSTDPAVFTDLASVDNSEFQQLLNQGIPVAPHTTEPMLMEYPEAITRLVTGAQ
RPPDPAPAPLGAPGLPNGLLSGDEDFSSIADMDFSALLGSGSGSRDSREGMFLPKPEAGSAISD
VFEGREVCQPKRIRPFHPPGSPWANRPLPASLAPTPTGPVHEPVGSLTPAPVPQPLDPAPAVTP
EASHLLEDPDEETSQAVKALREMADTVIPQKEEAAICGQMDLSHPPPRGHLDELTTTLESMTED
LNLDSPLTPELNEILDTFLNDECLLHAMHISTGLSIFDTSLFASGSGPKKKRKV
SEQ ID NO: 106 comprises the following SEQ ID NOS and spacers:
21-6-GSG-4-AGS-17-INSRSSGS-4-G-100-GSGSGS-15-ASGSG-4; where GSG, AGS,
INSRSSGS, G, GSGSGS, and ASGSG are peptide linkers.
SEQ ID NO: 107 = GCPp6: MCP-XTEN80-p65
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSSQYLPDTDDRHRIEEKRKRTYETFKSIMKKSPF
SGPTDPRPPPRRIAVPSRSSASVPKPAPQPYPFTSSLSTINYDEFPTMVFPSGQISQASALAPA
PPQVLPQAPAPAPAPAMVSALAQAPAPVPVLAPGPPQAVAPPAPKPTQAGEGTLSEALLQLQFD
DEDLGALLGNSTDPAVFTDLASVDNSEFQQLLNQGIPVAPHTTEPMLMEYPEAITRLVTGAQRP
PDPAPAPLGAPGLPNGLLSGDEDFSSIADMDFSALLASGSGPKKKRKV
SEQ ID NO: 107 comprises the following SEQ ID NOS and spacers:
21-6-GSG-4-AGS-100-ASGSG-4; where GSG, AGS, and ASGSG are peptide
linkers.
SEQ ID NO: 108 = GCPp7: MCP-XTEN80-Rta
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSRDSREGMFLPKPEAGSAISDVFEGREVCQPKRI
RPFHPPGSPWANRPLPASLAPTPTGPVHEPVGSLTPAPVPQPLDPAPAVTPEASHLLEDPDEET
SQAVKALREMADTVIPQKEEAAICGQMDLSHPPPRGHLDELTTTLESMTEDLNLDSPLTPELNE
ILDTFLNDECLLHAMHISTGLSIFDTSLFASGSGPKKKRKV
SEQ ID NO: 108 comprises the following SEQ ID NOS and spacers:
21-6-GSG-4-AGS-15-ASGSG-4; where GSG, AGS, and ASGSG are peptide
linkers.
SEQ ID NO: 109 = GCPp8: MCP-XTEN80-p65-Rta
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSSQYLPDTDDRHRIEEKRKRTYETFKSIMKKSPF
SGPTDPRPPPRRIAVPSRSSASVPKPAPQPYPFTSSLSTINYDEFPTMVFPSGQISQASALAPA
PPQVLPQAPAPAPAPAMVSALAQAPAPVPVLAPGPPQAVAPPAPKPTQAGEGTLSEALLQLQFD
DEDLGALLGNSTDPAVFTDLASVDNSEFQQLLNQGIPVAPHTTEPMLMEYPEAITRLVTGAQRP
PDPAPAPLGAPGLPNGLLSGDEDFSSIADMDFSALLGSGSGSRDSREGMFLPKPEAGSAISDVF
EGREVCQPKRIRPFHPPGSPWANRPLPASLAPTPTGPVHEPVGSLTPAPVPQPLDPAPAVTPEA
SHLLEDPDEETSQAVKALREMADTVIPQKEEAAICGQMDLSHPPPRGHLDELTTTLESMTEDLN
LDSPLTPELNEILDTFLNDECLLHAMHISTGLSIFDTSLFASGSGPKKKRKV
SEQ ID NO: 109 comprises the following SEQ ID NOS and spacers:
21-6-GSG-4-AGS-100-GSGSGS-15-ASGSG-4; where GSG, AGS, GSGSGS, and
ASGSG are peptide linkers.
SEQ ID NO: 110 = GCPp9: MCP-XTEN80-NLS
MASNFTQFVLVDNGGTGDVTVAPSNFANGVAEWISSNSRSQAYKVTCSVRQSSAQKRKYTIKVE
VPKVATQTVGGVELPVAAWRSYLNMELTIPIFATNSDCELIVKAMQGLLKDGNPIPSAIAANSG
IYGGPSSGAPPPSGGSPAGSPTSTEEGTSESATPESGPGTSTEPSEGSAPGSPAGSPTSTEEGT
STEPSEGSAPGTSTEPSEGSGPKKKRKVAGSASGSGPKKKRKV
SEQ ID NO: 110 comprises the following SEQ ID NOS and spacers:
21-6-GSG-4-AGSASGSG-4; where GSG and AGSASGSG are peptide linkers.
SEQ ID NO: 111 = GCPp11: dCas9-XTEN16-TET1
MDKKYSIGLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGNIVDEVAY
HEKYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKAPLSAS
MIKRYDEHHQDLTLLKALVRQQLPEKYKEIFFDQSKNGYAGYIDGGASQEEFYKFIKPILEKMD
GTEELLVKLNREDLLRKQRTFDNGSIPHQIHLGELHAILRRQEDFYPFLKDNREKIEKILTFRI
PYYVGPLARGNSRFAWMTRKSEETITPWNFEEVVDKGASAQSFIERMTNFDKNLPNEKVLPKHS
LLYEYFTVYNELTKVKYVTEGMRKPAFLSGEQKKAIVDLLFKTNRKVTVKQLKEDYFKKIECFD
SVEISGVEDRFNASLGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFANRNFMQLIHDDSLTF
KEDIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDINR
LSDYDVDAIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRKMIAK
SEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSKRV
ILADANLDKVLSAYNKHRDKPIREQAENIIHLFTLTNLGAPAAFKYFDTTIDRKRYTSTKEVLD
ATLIHQSITGLYETRIDLSQLGGDGGGGSPKKKRKVDPKKKRKVDPKKKRKVGSGSETPGTSES
ATPESSLPTCSCLDRVIQKDKGPYYTHLGAGPSVAAVREIMENRYGQKGNAIRIEIVVYTGKEG
KSSHGCPIAKWVLRRSSDEEKVLCLVRQRTGHHCPTAVMVVLIMVWDGIPLPMADRLYTELTEN
LKSYNGHPTDRRCTLNENRTCTCQGIDPETCGASFSFGCSWSMYFNGCKFGRSPSPRRFRIDPS
SPLHEKNLEDNLQSLATRLAPIYKQYAPVAYQNQVEYENVARECRLGSKEGRPFSGVTACLDFC
AHPHRDIHNMNNGSTVVCTLTREDNRSLGVIPQDEQLHVLPLYKLSDTDEFGSKEGMEAKIKSG
AIEVLAPRRKKRTCFTQPVPRSGKKRAAMMTEVLAHKIRAVEKKPIPRIKRKNNSTTTNNSKPS
SLPTLGSNTETVQPEVKSETEPHFILKSSDNTKTYSLMPSAPHPVKEASPGFSWSPKTASATPA
PLKNDATASCGFSERSSTPHCTMPSGRLSGANAAAADGPGISQLGEVAPLPTLSAPVMEPLINS
EPSTGVTEPLTPHQPNHQPSFLTSPQDLASSPMEEDEQHSEADEPPSDEPLSDDPLSPAEEKLP
HIDEYWSDSEHIFLDANIGGVAIAPAHGSVLIECARRELHATTPVEHPNRNHPTRLSLVFYQHK
NLNKPQHGFELNKIKFEAKEAKNKKMKASEQKDQAANEGPEQSSEVNELNQIPSHKALTLTHDN
VVTVSPYALTHVAGPYNHWV
SEQ ID NO: 111 comprises the following SEQ ID NOS and spacers:
9-GGGGS-4-D-4-D-4-G-5-86; where GGGGS, D, D, and G are peptide linkers.
SEQ ID NO: 112 = GCPp16: TET1-XTEN16-dCas9
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWVSGSETPGTSESATPESMDKKYSIGLAIGTNSVGWAVITDEYKVPSKKF
KVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDDSIDNKVLTRSDKNR
GKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGDGGGGSPKK
KRKVDPKKKRKVDPKKKRKV
SEQ ID NO: 112 comprises the following SEQ ID NOS and spacers:
97-5-9-GGGGS-4-D-4-D-4; where GGGGS, D, and D are peptide linkers.
SEQ ID NO: 113 = GCP20: TET1-XTEN80-dCas9
MALPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLKSY
NGHPTDRRCTLNENRTCTCQGIDPETCGASFSFGCSWSMYFNGCKFGRSPSPRRFRIDPSSPLH
EKNLEDNLQSLATRLAPIYKQYAPVAYQNQVEYENVARECRLGSKEGRPFSGVTACLDFCAHPH
RDIHNMNNGSTVVCTLTREDNRSLGVIPQDEQLHVLPLYKLSDTDEFGSKEGMEAKIKSGAIEV
LAPRRKKRTCFTQPVPRSGKKRAAMMTEVLAHKIRAVEKKPIPRIKRKNNSTTTNNSKPSSLPT
LGSNTETVQPEVKSETEPHFILKSSDNTKTYSLMPSAPHPVKEASPGFSWSPKTASATPAPLKN
DATASCGFSERSSTPHCTMPSGRLSGANAAAADGPGISQLGEVAPLPTLSAPVMEPLINSEPST
GVTEPLTPHQPNHQPSFLTSPQDLASSPMEEDEQHSEADEPPSDEPLSDDPLSPAEEKLPHIDE
YWSDSEHIFLDANIGGVAIAPAHGSVLIECARRELHATTPVEHPNRNHPTRLSLVFYQHKNLNK
PQHGFELNKIKFEAKEAKNKKMKASEQKDQAANEGPEQSSEVNELNQIPSHKALTLTHDNVVTV
SPYALTHVAGPYNHWVGGPSSGAPPPSGGSPAGSPTSTEEGTSESATPESGPGTSTEPSEGSAP
GSPAGSPTSTEEGTSTEPSEGSAPGTSTEPSEMDKKYSIGLAIGTNSVGWAVITDEYKVPSKKF
KVLGNTDRHSIKKNLIGALLFDSGETAEATRLKRTARRRYTRRKNRICYLQEIFSNEMAKVDDS
FFHRLEESFLVEEDKKHERHPIFGNIVDEVAYHEKYPTIYHLRKKLVDSTDKADLRLIYLALAH
MIKFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLKALVRQQLPEKYKEIFF
DQSKNGYAGYIDGGASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDDSIDNKVLTRSDKNR
GKSDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLANGE
IRKRPLIETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGDGGGGSPKK
KRKVDPKKKRKVDPKKKRKV
SEQ ID NO: 113 comprises the following SEQ ID NOS and spacers:
97-6-9-GGGGS-4-D-4-D-4; where GGGGS, D, and D are peptide linkers.
SEQ ID NO: 114
GACGCTCAAATTTCCGCAGTGTTTAAGAGCTAAGCTGGAAACAGCATAGCAAGTTT
AAATAAGGCTAGTCCGTTATCAACTTGAAAAAGTGGCACCGAGTCGGTGCTTTTTTT
SEQ ID NO: 115
GTTTAAGAGCTAAGCTGGAAACAGCATAGCAAGTTTAAATAAGGCTAGTCCGTTAT
CAACTTGAAAAAGTGGCACCGAGTCGGTGCTTTTTTT
SEQ ID NO: 116
GACGCTCAAATTTCCGCAGT
SEQ ID NO: 117 (DNA sequence encoding the MS2-sgRNA scaffold)
5′-
GTTTAAGAGCTAaGCCAACATGAGGATCACCCATGTCTGCAGGGCaTAGCAAGTTTA
AATAAGGCTAGTCCGTTATCAACTTGGCCAACATGAGGATCACCCATGTCTGCAGG
GCCAAGTGGCACCGAGTCGGTGCTTTTTTT-3′
SEQ ID NO: 118 (T7 promoter sequence)
5′-TAATACGACTCACTATAGG-3′
SEQ ID NO: 119
AGATCGGAAGAGCACACGTCTGAACTC

1. A fusion protein comprising from N-terminus to C-terminus, a demethylation domain, an XTEN linker, and a nuclease-deficient RNA-guided DNA endonuclease enzyme.

2. The fusion protein of claim 1, wherein the demethylation domain is a TET1 domain, a TET2 domain, a TET3 domain, or a combination of two or more thereof.

3. The fusion protein of claim 2, wherein the demethylation domain is a TET1 domain.

4. The fusion protein of claim 2, wherein the TET1 domain comprises an amino acid sequence having at least 90% sequence identity to SEQ ID NO:1, SEQ ID NO:86, or SEQ ID NO:97.

5. The fusion protein of claim 1, wherein the nuclease-deficient RNA-guided DNA endonuclease enzyme is dCas9, dCas12a, dCpf1, Cas-phi, a helix-turn-helix motif, a helix-loop-helix domain, an HMB-box domain, a Wor3 domain, an OB-fold domain, an immunoglobulin domain, or a B3 domain.

6. The fusion protein of claim 5, wherein the nuclease-deficient RNA-guided DNA endonuclease enzyme is dCas9.

7. The fusion protein of claim 1, wherein the XTEN linker comprises from about 10 amino acid residues to about 864 amino acid residues.

8. The fusion protein of claim 7, wherein the XTEN linker comprises an amino acid sequence having at least 90% sequence identity to SEQ ID NO:5, SEQ ID NO:6 or SEQ ID NO:98.

9. The fusion protein of claim 1, wherein the fusion protein further comprises an epitope tag, a 2A peptide, a fluorescent protein tag, a nuclear localization signal peptide, or a combination of two or more thereof.

10. A fusion protein comprising from N-terminus to C-terminus, an RNA-binding sequence, an XTEN linker, and at least one transcriptional activator.

11. The fusion protein of claim 10, wherein the transcriptional activator is VP64, p65, Rta, or a combination of two or more thereof.

12. The fusion protein of claim 11, wherein p65 comprises an amino acid sequence having at least 90% sequence identity to SEQ ID NO:13, SEQ ID NO:14, or SEQ ID NO:100.

13. The fusion protein of claim 11, wherein Rta comprises an amino acid sequence having at least 90% sequence identity to SEQ ID NO:15 or SEQ ID NO:16.

14. The fusion protein of claim 11, wherein VP64 comprises an amino acid sequence having at least 90% sequence identity to SEQ ID NO:17 or SEQ ID NO:18.

15. The fusion protein of claim 10, wherein the RNA-binding sequence is an MS2 RNA-binding sequence.

16. The fusion protein of claim 15, wherein the MS2 RNA-binding sequence comprises the amino acid sequence of SEQ ID NO:21.

17. The fusion protein of claim 10, wherein the XTEN linker comprises from about 10 amino acid residues to about 864 amino acid residues.

18. The fusion protein of claim 10 having an amino acid sequence with at least 90% sequence identity to SEQ ID NO:104, SEQ ID NO:105, SEQ ID NO:106, SEQ ID NO:107, SEQ ID NO:108, SEQ ID NO:109, or SEQ ID NO:110.

19. The fusion protein of claim 10, wherein the fusion protein further comprises an epitope tag, a 2A peptide, a fluorescent protein tag, a nuclear localization signal peptide, or a combination of two or more thereof.

20. A fusion protein comprising from N-terminus to C-terminus, a demethylation domain, a first XTEN linker, a nuclease-deficient RNA-guided DNA endonuclease enzyme, a second XTEN linker, and a transcriptional activator.

21. The fusion protein of claim 20, wherein the transcriptional activator is VP64, p65, Rta, or a combination of two or more thereof.

22. A fusion protein comprising from N-terminus to C-terminus, a demethylation domain, an XTEN linker, and a nuclease-deficient RNA-guided DNA endonuclease enzyme.

23. The fusion protein of claim 20, further comprising a nuclear localization sequence

24. The fusion protein of claim 20, wherein the demethylation domain is a TET1 domain, a TET2 domain, a TET3 domain, or a combination of two or more thereof.

25. The fusion protein of claim 24, wherein the demethylation domain is a TET1 domain.

26. The fusion protein of claim 20, wherein the nuclease-deficient RNA-guided DNA endonuclease enzyme is dCas9, dCas12a, dCpf1, Cas-phi, a leucine zipper domain, a winged helix domain, a helix-turn-helix motif, a helix-loop-helix domain, an HMB-box domain, a Wor3 domain, an OB-fold domain, an immunoglobulin domain, or a B3 domain.

27. The fusion protein of claim 26, wherein the nuclease-deficient RNA-guided DNA endonuclease enzyme is dCas9.

28. The fusion protein of claim 20, wherein the first XTEN linker and the second XTEN linker each independently comprise from about 10 amino acid residues to about 864 amino acid residues.

29. The fusion protein of claim 20, wherein the fusion protein further comprising an epitope tag, a 2A peptide, a fluorescent protein tag, or a combination of two or more thereof.

30. A fusion protein comprising an amino acid sequence having at least 90% sequence identity to SEQ ID NO:99, SEQ ID NO:101, SEQ ID NO:102, SEQ ID NO:111, SEQ ID NO:112, or SEQ ID NO:113.

31. The fusion protein of claim 30, comprising an amino acid sequence having at least 95% sequence identity to SEQ ID NO:99, SEQ ID NO:101, SEQ ID NO:102, SEQ ID NO:111, SEQ ID NO:112, or SEQ ID NO:113.

32. The fusion protein of claim 31 comprising SEQ ID NO:99, SEQ ID NO:101, SEQ ID NO:102, SEQ ID NO:111, SEQ ID NO:112, or SEQ ID NO:113.

33. A method of activating or reactivating a target nucleic acid sequence in a cell, the method comprising:
delivering a first polynucleotide encoding a fusion protein of claim 1 to a cell containing the target nucleic acid; and
(ii) delivering to the cell a second polynucleotide comprising: (a) a sgRNA or (b) a cr:tracrRNA;
thereby activating or reactivating the target nucleic acid sequence in the cell.

34. The method of claim 32, wherein the target nucleic acid sequence comprises a CpG island.

35. The method of claim 32, wherein the target nucleic acid sequence comprises a non-CpG island.

36. The method of claim 32, wherein the second polynucleotide comprises the sgRNA.

37. The method of claim 32, wherein the sgRNA comprises at least one MS2 stem loop.

38. The method of claim 37, wherein the sgRNA comprises two MS2 stem loops.

39. The method of claim 32, wherein the second polynucleotide encodes a transcriptional activator.

40. The method of claim 39, wherein the transcriptional activator is VP64, p65, Rta, or a combination of two or more thereof.

41. The method of claim 32, wherein the second polynucleotide further encodes an MS2 RNA-binding sequence.

42. The method of claim 41, wherein the MS2 RNA-binding sequence comprises the amino acid sequence of SEQ ID NO:21.

43. The method of claim 32, wherein the second polynucleotide further encodes for an XTEN linker, an epitope tag, a 2A peptide, a fluorescent protein tag, a nuclear localization signal peptide, or a combination of two or more thereof.

44. The method of claim 32, further comprising delivering to the cell a third polynucleotide encoding a second fusion protein which comprises a transcriptional activator.

45. The method of claim 44, wherein the transcriptional activator is VP64, p65, Rta, or a combination of two or more thereof.

46. The method of claim 44, wherein the second fusion protein further comprises an MS2 RNA-binding sequence.

47. The method of claim 46, wherein the MS2 RNA-binding sequence comprises the amino acid sequence of SEQ ID NO:21.

48. The method of claim 44, wherein the second fusion protein further comprises an XTEN linker, an epitope tag, a 2A peptide, a fluorescent protein tag, a nuclear localization signal peptide, or a combination of two or more thereof.

49. A fusion protein comprising from N-terminus to C-terminus, a demethylation domain, an XTEN linker, and a nuclease-deficient DNA endonuclease enzyme.

50. The fusion protein of claim 49, wherein the demethylation domain is a TET1 domain, a TET2 domain, a TET3 domain, or a combination of two or more thereof.

51. The fusion protein of claim 49, wherein the demethylation domain is a TET1 domain.

52. The fusion protein of claim 51, wherein the TET1 domain comprises an amino acid sequence having at least 90% sequence identity to SEQ ID NO:1, SEQ ID NO:86, or SEQ ID NO:97.

53. The fusion protein of claim 49, wherein the nuclease-deficient DNA endonuclease enzyme is a zinc finger domain.

54. The fusion protein of claim 49, wherein the nuclease-deficient DNA endonuclease enzyme is a TALE.

55. The fusion protein of claim 49, wherein the XTEN linker comprises from about 10 amino acid residues to about 864 amino acid residues.

56. The fusion protein of claim 55, wherein the XTEN linker comprises an amino acid sequence having at least 90% sequence identity to SEQ ID NO:5, SEQ ID NO:6 or SEQ ID NO:98.

57. The fusion protein of claim 49, wherein the fusion protein further comprises an epitope tag, a 2A peptide, a fluorescent protein tag, a nuclear localization signal peptide, or a combination of two or more thereof.

58. A fusion protein comprising from N-terminus to C-terminus, a demethylation domain, a first XTEN linker, a nuclease-deficient DNA endonuclease enzyme, a second XTEN linker, and a transcriptional activator.

59. The fusion protein of claim 58, wherein the transcriptional activator is VP64, p65, Rta, or a combination of two or more thereof.

60. A fusion protein comprising from N-terminus to C-terminus, a demethylation domain, an XTEN linker, and a nuclease-deficient DNA endonuclease enzyme.

61. The fusion protein of claim 58, further comprising a nuclear localization sequence.

62. The fusion protein of claim 58, wherein the demethylation domain is a TET1 domain, a TET2 domain, a TET3 domain, or a combination of two or more thereof.

63. The fusion protein of claim 62, wherein the demethylation domain is a TET1 domain.

64. The fusion protein of claim 58, wherein the nuclease-deficient DNA endonuclease enzyme is a zinc finger domain.

65. The fusion protein of claim 58, wherein the nuclease-deficient DNA endonuclease enzyme is a TALE.

66. The fusion protein of claim 58, wherein the first XTEN linker and the second XTEN linker each independently comprise from about 10 amino acid residues to about 864 amino acid residues.

67. The fusion protein of claim 58, wherein the fusion protein further comprising an epitope tag, a 2A peptide, a fluorescent protein tag, or a combination of two or more thereof.

68. A method of activating or reactivating a target nucleic acid sequence in a cell, the method comprising delivering a polynucleotide encoding a fusion protein of claim 58 to a cell containing the target nucleic acid; thereby activating or reactivating the target nucleic acid sequence in the cell.

69. The method of claim 68, wherein the transcriptional activator is VP64, p65, Rta, or a combination of two or more thereof.


SEQUENCE LISTING (120 sequences)

SEQ ID NO: 1 - protein, 2136 aa, Homo sapiens
MSRSRHARPSRLVRKEDVNKKKKNSQLRKTTKGANKNVASVKTLSPGKLKQLIQERDVKKKTEPKPPVPVRSLLTRAGAARMNLDRTEVLFQNPESLTCNGFTMALRSTSLSRRLSQPPLVVAKSKKVPLSKGLEKQHDCDYKILPALGVKHSENDSVPMQDTQVLPDIETLIGVQNPSLLKGKSQETTQFWSQRVEDSKINIPTHSGPAAEILPGPLEGTRCGEGLFSEETLNDTSGSPKMFAQDTVCAPFPQRATPKVTSQGNPSIQLEELGSRVESLKLSDSYLDPIKSEHDCYPTSSLNKVIPDLNLRNCLALGGSTSPTSVIKFLLAGSKQATLGAKPDHQEAFEATANQQEVSDTTSFLGQAFGAIPHQWELPGADPVHGEALGETPDLPEIPGAIPVQGEVFGTILDQQETLGMSGSVVPDLPVFLPVPPNPIATFNAPSKWPEPQSTVSYGLAVQGAIQILPLGSGHTPQSSSNSEKNSLPPVMAISNVENEKQVHISFLPANTQGFPLAPERGLFHASLGIAQLSQAGPSKSDRGSSQVSVTSTVHVVNTTVVTMPVPMVSTSSSSYTTLLPTLEKKKRKRCGVCEPCQQKTNCGECTYCKNRKNSHQICKKRKCEELKKKPSVVVPLEVIKENKRPQREKKPKVLKADFDNKPVNGPKSESMDYSRCGHGEEQKLELNPHTVENVTKNEDSMTGIEVEKWTQNKKSQLTDHVKGDFSANVPEAEKSKNSEVDKKRTKSPKLFVQTVRNGIKHVHCLPAETNVSFKKFNIEEFGKTLENNSYKFLKDTANHKNAMSSVATDMSCDHLKGRSNVLVFQQPGFNCSSIPHSSHSIINHHASIHNEGDQPKTPENIPSKEPKDGSPVQPSLLSLMKDRRLTLEQVVAIEALTQLSEAPSENSSPSKSEKDEESEQRTASLLNSCKAILYTVRKDLQDPNLQGEPPKLNHCPSLEKQSSCNTVVFNGQTTTLSNSHINSATNQASTKSHEYSKVTNSLSLFIPKSNSSKIDTNKSIAQGIITLDNCSNDLHQLPPRNNEVEYCNQLLDSSKKLDSDDLSCQDATHTQIEEDVATQLTQLASIIKINYIKPEDKKVESTPTSLVTCNVQQKYNQEKGTIQQKPPSSVHNNHGSSLTKQKNPTQKKTKSTPSRDRRKKKPTVVSYQENDRQKWEKLSYMYGTICDIWIASKFQNFGQFCPHDFPTVFGKISSSTKIWKPLAQTRSIMQPKTVFPPLTQIKLQRYPESAEEKVKVEPLDSLSLFHLKTESNGKAFTDKAYNSQVQLTVNANQKAHPLTQPSSPPNQCANVMAGDDQIRFQQVVKEQLMHQRLPTLPGISHETPLPESALTLRNVNVVCSGGITVVSTKSEEEVCSSSFGTSEFSTVDSAQKNFNDYAMNFFTNPTKNLVSITKDSELPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 2 - protein, 1942 aa, Homo sapiens
YGIPCMKGSQNSRVSPDFTQESRGYSKCLQNGGIKRTVSEPSLSGLLQIKKLKQDQKANGERRNFGVSQERNPGESSQPNVSDLSDKKESVSSVAQENAVKDFTSFSTHNCSGPENPELQILNEQEGKSANYHDKNIVLLKNKAVLMPNGATVSASSVEHTHGELLEKTLSQYYPDCVSIAVQKTTSHINAINSQATNELSCEITHPSHTSGQINSAQTSNSELPPKPAAVVSEACDADDADNASKLAAMLNTCSFQKPEQLQQQKSVFEICPSPAENNIQGTTKLASGEEFCSGSSSNLQAPGGSSERYLKQNEMNGAYFKQSSVFTKDSFSATTTPPPPSQLLLSPPPPLPQVPQLPSEGKSTLNGGVLEEHHHYPNQSNTTLLREVKIEGKPEAPPSQSPNPSTHVCSPSPMLSERPQNNCVNRNDIQTAGTMTVPLCSEKTRPMSEHLKHNPPIFGSSGELQDNCQQLMRNKEQEILKGRDKEQTRDLVPPTQHYLKPGWIELKAPRFHQAESHLKRNEASLPSILQYQPNLSNQMTSKQYTGNSNMPGGLPRQAYTQKTTQLEHKSQMYQVEMNQGQSQGTVDQHLQFQKPSHQVHFSKTDHLPKAHVQSLCGTRFHFQQRADSQTEKLMSPVLKQHLNQQASETEPFSNSHLLQHKPHKQAAQTQPSQSSHLPQNQQQQQKLQIKNKEEILQTFPHPQSNNDQQREGSFFGQTKVEECFHGENQYSKSSEFETHNVQMGLEEVQNINRRNSPYSQTMKSSACKIQVSCSNNTHLVSENKEQTTHPELFAGNKTQNLHHMQYFPNNVIPKQDLLHRCFQEQEQKSQQASVLQGYKNRNQDMSGQQAAQLAQQRYLIHNHANVFPVPDQGGSHTQTPPQKDTQKHAALRWHLLQKQEQQQTQQPQTESCHSQMHRPIKVEPGCKPHACMHTAPPENKTWKKVTKQENPPASCDNVQQKSIIETMEQHLKQFHAKSLFDHKALTLKSQKQVKVEMSGPVTVLTRQTTAAELDSHTPALEQQTTSSEKTPTKRTAASVLNNFIESPSKLLDTPIKNLLDTPVKTQYDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 3 - protein, 1795 aa, Homo sapiens
MSQFQVPLAVQPDLPGLYDFPQRQVMVGSFPGSGLSMAGSESQLRGGGDGRKKRKRCGTCEPCRRLENCGACTSCTNRRTHQICKLRKCEVLKKKVGLLKEVEIKAGEGAGPWGQGAAVKTGSELSPVDGPVPGQMDSGPVYHGDSRQLSASGVPVNGAREPAGPSLLGTGGPWRVDQKPDWEAAPGPAHTARLEDAHDLVAFSAVAEAVSSYGALSTRLYETFNREMSREAGNNSRGPRPGPEGCSAGSEDLDTLQTALALARHGMKPPNCNCDGPECPDYLEWLEGKIKSVVMEGGEERPRLPGPLPPGEAGLPAPSTRPLLSSEVPQISPQEGLPLSQSALSIAKEKNISLQTAIAIEALTQLSSALPQPSHSTPQASCPLPEALSPPAPFRSPQSYLRAPSWPVVPPEEHSSFAPDSSAFPPATPRTEFPEAWGTDTPPATPRSSWPMPRPSPDPMAELEQLLGSASDYIQSVFKRPEALPTKPKVKVEAPSSSPAPAPSPVLQREAPTPSSEPDTHQKAQTALQQHLHHKRSLFLEQVHDTSFPAPSEPSAPGWWPPPSSPVPRLPDRPPKEKKKKLPTPAGGPVGTEKAAPGIKPSVRKPIQIKKSRPREAQPLFPPVRQIVLEGLRSPASQEVQAHPPAPLPASQGSAVPLPPEPSLALFAPSPSRDSLLPPTQEMRSPSPMTALQPGSTGPLPPADDKLEELIRQFEAEFGDSFGLPGPPSVPIQDPENQQTCLPAPESPFATRSPKQIKIESSGAVTVLSTTCFHSEEGGQEATPTKAENPLTPTLSGFLESPLKYLDTPTKSLLDTPAKRAQAEFPTCDCVEQIVEKDEGPYYTHLGSGPTVASIRELMEERYGEKGKAIRIEKVIYTGKEGKSSRGCPIAKWVIRRHTLEEKLLCLVRHRAGHHCQNAVIVILILAWEGIPRSLGDTLYQELTDTLRKYGNPTSRRCGLNDDRTCACQGKDPNTCGASFSFGCSWSMYFNGCKYARSKTPRKFRLAGDNPKEEEVLRKSFQDLATEVAPLYKRLAPQAYQNQVTNEEIAIDCRLGLKEGRPFAGVTACMDFCAHAHKDQHNLYNGCTVVCTLTKEDNRCVGKIPEDEQLHVLPLYKMANTDEFGSEENQNAKVGSGAIQVLTAFPREVRRLPEPAKSCRQRQLEARKAAAEKKKIQKEKLSTPEKIKQEALELAGITSDPGLSLKGGLSQQGLKPSLKVEPQNHFSSFKYSGNAVVESYSVLGNCRPSDPYSMNSVYSYHSYYAQPSLTSVNGFHSKYALPSFSYYGFPSSNPVFPSQFLGPGAWGHSGSSGSFEKKPDLHALHNSLSPAYGGAEFAELPSQAVPTDAHHPTPHHQQPAYPGPKEYLLPKAPLLHSVSRDPSPFAQSSNCYNRSIKQEPVDPLTQAEPVPRDAGKMGKTPLSEVSQNGGPSHLWGQYSGGPSMSPKRTNGVGGSWGVFSSGESPAIVPDKLSSFGASCLAPSHFTDGQWGLFPGEGQQAASHSGGRLRGKPWSPCKFGNSTSALAGPSLTEKPWALGAGDFNSALKGSPGFQDKLWNPMKGEEGRIPAAGASQLDRAWQSFGLPLGSSEKLFGALKSEEKLWDPFSLEEGPAEEPPSKGAVKEEKGGGGAEEEEEELWSDSEHNFLDENIGGVAVAPAHGSILIECARRELHATTPLKKPNRCHPTRISLVFYQHKNLNQPNHGLALWEAKMKQLAERARARQEEAARLGLGQQEAKLYGKKRKWGGTVVAEPQQKEKKGVVPTRQALAVPTDSAVTVSSYAYTKVTGPYSRWI

SEQ ID NO: 4 - protein, 7 aa, Artificial Sequence
  Synthetic polypeptide
PKKKRKV

SEQ ID NO: 5 - protein, 16 aa, Artificial Sequence
  Synthetic polypeptide
SGSETPGTSESATPES

SEQ ID NO: 6 - protein, 80 aa, Artificial Sequence
  Synthetic polypeptide
GGPSSGAPPPSGGSPAGSPTSTEEGTSESATPESGPGTSTEPSEGSAPGSPAGSPTSTEEGTSTEPSEGSAPGTSTEPSE

SEQ ID NO: 7 - protein, 9 aa, Artificial Sequence
  Synthetic polypeptide
YPYDVPDYA

SEQ ID NO: 8 - protein, 232 aa, Artificial Sequence
  Synthetic polypeptide
SELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 9 - protein, 1368 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 10 - protein, 1307 aa, Artificial Sequence
  Synthetic polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLANLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 11 - protein, 1228 aa, Artificial Sequence
  Synthetic polypeptide
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIARGERNLLYIVVVDGKGNIVEQYSLNEIINNFNGIRIKTDYHSLLDKKEKERFEARQNWTSIENIKELKAGYISQVVHKICELVEKYDAVIALADLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 12 - protein, 1314 aa, Artificial Sequence
  Synthetic polypeptide
MYPYDVPDYASGSGMSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 13 - protein, 551 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 14 - protein, 119 aa, Pan paniscus
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 15 - protein, 190 aa, Human gammaherpesvirus 4
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 16 - protein, 605 aa, Human gammaherpesvirus 4
MRPKKDGLEDFLRLTPEIKKQLGSLVSDYCNVLNKEFTAGSVEITLRSYKICKAFINEAKAHGREWGGLMATLNICNFWAILRNNRVRRRAENAGNDACSIACPIVMRYVLDHLIVVTDRFFIQAPSNRVMIPATIGTAMYKLLKHSRVRAYTYSKVLGVDRAAIMASGKQVVEHLNRMEKEGLLSSKFKAFCKWVFTYPVLEEMFQTMVSSKTGHLTDDVKDVRALIKTLPRASYSSHAGQRSYVSGVLPACLLSTKSKAVETPILVSGADRMDEELMGNDGGASHTEARYSESGQFHAFTDELESLPSPTMPLKPGAQSADCGDSSSSSSDSGNSDTEQSEREEARAEAPRLRAPKSRRTSRPNRGQTPCPSNAAEPEQPWIAAVHQESDERPIFPHPSKPTFLPPVKRKKGLRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 17 - protein, 50 aa, Artificial Sequence
  Synthetic polypeptide
DALDDFDLDMLGSDALDDFDLDMLGSDALDDFDLDMLGSDALDDFDLDML

SEQ ID NO: 18 - protein, 490 aa, Human herpesvirus 1
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 19 - DNA, 33 bp, Artificial Sequence
  Synthetic polynucleotide
agccaacatgaggatcacccatgtctgcagggc

SEQ ID NO: 20 - DNA, 34 bp, Artificial Sequence
  Synthetic polynucleotide
ggccaacatgaggatcacccatgtctgcagggcc

SEQ ID NO: 21 - protein, 130 aa, Bacteriophage MS2
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 22 - DNA, 19 bp, Artificial Sequence
  Synthetic polynucleotide
actgcggaaatttgagcgt

SEQ ID NO: 23 - DNA, 19 bp, Artificial Sequence
  Synthetic polynucleotide
acgctcaaatttccgcagt

SEQ ID NO: 24 - DNA, 19 bp, Artificial Sequence
  Synthetic polynucleotide
aggcaatggctgcacatgc

SEQ ID NO: 25 - DNA, 19 bp, Artificial Sequence
  Synthetic polynucleotide
gcatgtgcagccattgcct

SEQ ID NO: 26 - DNA, 19 bp, Artificial Sequence
  Synthetic polynucleotide
gacgcttggttctgaggag

SEQ ID NO: 27 - DNA, 19 bp, Artificial Sequence
  Synthetic polynucleotide
ctcctcagaaccaagcgtc

SEQ ID NO: 28 - DNA, 19 bp, Artificial Sequence
  Synthetic polynucleotide
tccggaaacgcattcctct

SEQ ID NO: 29 - DNA, 19 bp, Artificial Sequence
  Synthetic polynucleotide
agaggaatgcgtttccgga

SEQ ID NO: 30 - DNA, 19 bp, Artificial Sequence
  Synthetic polynucleotide
ccgcgtcagcccggcccgg

SEQ ID NO: 31 - DNA, 19 bp, Artificial Sequence
  Synthetic polynucleotide
ccgggccgggctgacgcgg

SEQ ID NO: 32 - DNA, 19 bp, Artificial Sequence
  Synthetic polynucleotide
cgactcccgctgggcctct

SEQ ID NO: 33 - DNA, 19 bp, Artificial Sequence
  Synthetic polynucleotide
agaggcccagcgggagtcg

SEQ ID NO: 34 - DNA, 19 bp, Artificial Sequence
  Synthetic polynucleotide
ccgttgcgcgctcgctctc

SEQ ID NO: 35 - DNA, 19 bp, Artificial Sequence
  Synthetic polynucleotide
gagagcgagcgcgcaacgg

SEQ ID NO: 36 - DNA, 19 bp, Artificial Sequence
  Synthetic polynucleotide
ccgcgcatcctgccaggcc

SEQ ID NO: 37 - DNA, 19 bp, Artificial Sequence
  Synthetic polynucleotide
actgcggaaatttgagcgt

SEQ ID NO: 38 - DNA, 19 bp, Artificial Sequence
  Synthetic polynucleotide
acgcucaaauuuccgcagu

SEQ ID NO: 39 - DNA, 19 bp, Artificial Sequence
  Synthetic polynucleotide
aggcaatggctgcacatgc

SEQ ID NO: 40 - DNA, 19 bp, Artificial Sequence
  Synthetic polynucleotide
gcaugugcagccauugccu

SEQ ID NO: 41 - DNA, 19 bp, Artificial Sequence
  Synthetic polynucleotide
gacgcttggttctgaggag

SEQ ID NO: 42 - DNA, 19 bp, Artificial Sequence
  Synthetic polynucleotide
cuccucagaaccaagcguc

SEQ ID NO: 43 - DNA, 19 bp, Artificial Sequence
  Synthetic polynucleotide
tccggaaacgcattcctct

SEQ ID NO: 44 - DNA, 19 bp, Artificial Sequence
  Synthetic polynucleotide
agaggaaugcguuuccgga

SEQ ID NO: 45 - DNA, 19 bp, Artificial Sequence
  Synthetic polynucleotide
ccgcgtcagcccggcccgg

SEQ ID NO: 46 - DNA, 19 bp, Artificial Sequence
  Synthetic polynucleotide
ccgggccgggcugacgcgg

SEQ ID NO: 47 - DNA, 19 bp, Artificial Sequence
  Synthetic polynucleotide
cgactcccgctgggcctct

SEQ ID NO: 48 - DNA, 19 bp, Artificial Sequence
  Synthetic polynucleotide
agaggcccagcgggagucg

SEQ ID NO: 49 - DNA, 19 bp, Artificial Sequence
  Synthetic polynucleotide
ccgttgcgcgctcgctctc

SEQ ID NO: 50 - DNA, 19 bp, Artificial Sequence
  Synthetic polynucleotide
gagagcgagcgcgcaacgg

SEQ ID NO: 51 - DNA, 19 bp, Artificial Sequence
  Synthetic polynucleotide
ccgcgcatcctgccaggcc

SEQ ID NO: 52 - DNA, 19 bp, Artificial Sequence
  Synthetic polynucleotide
ggccuggcaggaugcgcgg

SEQ ID NO: 53 - DNA, 19 bp, Artificial Sequence
  Synthetic polynucleotide
ccaacttggcgcgtttcgg

SEQ ID NO: 54 - DNA, 19 bp, Artificial Sequence
  Synthetic polynucleotide
ccgaaacgcgccaaguugg

SEQ ID NO: 55 - DNA, 19 bp, Artificial Sequence
  Synthetic polynucleotide
accacgcgtccgagtccgg

SEQ ID NO: 56 - DNA, 19 bp, Artificial Sequence
  Synthetic polynucleotide
ccggacucggacgcguggu

SEQ ID NO: 57 - DNA, 19 bp, Artificial Sequence
  Synthetic polynucleotide
tgctcattgtccctggaca

SEQ ID NO: 58 - DNA, 19 bp, Artificial Sequence
  Synthetic polynucleotide
uguccagggacaaugagca

SEQ ID NO: 59 - DNA, 19 bp, Artificial Sequence
  Synthetic polynucleotide
ggacaccctgctcattgtc

SEQ ID NO: 60 - DNA, 19 bp, Artificial Sequence
  Synthetic polynucleotide
gacaaugagcagggugucc

SEQ ID NO: 61 - DNA, 19 bp, Artificial Sequence
  Synthetic polynucleotide
accggcagcctgcgcgtcc

SEQ ID NO: 62 - DNA, 19 bp, Artificial Sequence
  Synthetic polynucleotide
ggacgcgcaggcugccggu

SEQ ID NO: 63 - DNA, 19 bp, Artificial Sequence
  Synthetic polynucleotide
cgatgggcacccactgctc

SEQ ID NO: 64 - DNA, 19 bp, Artificial Sequence
  Synthetic polynucleotide
gagcagugggugcccaucg

SEQ ID NO: 65 - DNA, 19 bp, Artificial Sequence
  Synthetic polynucleotide
ccttcacgtggacgcgcag

SEQ ID NO: 66 - DNA, 19 bp, Artificial Sequence
  Synthetic polynucleotide
cugcgcguccacgugaagg

SEQ ID NO: 67 - DNA, 19 bp, Artificial Sequence
  Synthetic polynucleotide
cgtgaaggtggaagccttc

SEQ ID NO: 68 - DNA, 19 bp, Artificial Sequence
  Synthetic polynucleotide
gaaggcuuccaccuucacg

SEQ ID NO: 69 - DNA, 19 bp, Artificial Sequence
  Synthetic polynucleotide
ctccttggtcaggcgccgg

SEQ ID NO: 70 - DNA, 19 bp, Artificial Sequence
  Synthetic polynucleotide
ccggcgccugaccaaggag

SEQ ID NO: 71 - DNA, 19 bp, Artificial Sequence
  Synthetic polynucleotide
tggtcaggcgccggttccg

SEQ ID NO: 72 - DNA, 19 bp, Artificial Sequence
  Synthetic polynucleotide
cggaaccggcgccugacca

SEQ ID NO: 73 - DNA, 19 bp, Artificial Sequence
  Synthetic polynucleotide
tagaggtcgccttctcctc

SEQ ID NO: 74 - DNA, 19 bp, Artificial Sequence
  Synthetic polynucleotide
gaggagaaggcgaccucua

SEQ ID NO: 75 - DNA, 19 bp, Artificial Sequence
  Synthetic polynucleotide
cgacgctcgggtcgcggtg

SEQ ID NO: 76 - DNA, 19 bp, Artificial Sequence
  Synthetic polynucleotide
caccgcgacccgagcgucg

SEQ ID NO: 77 - DNA, 19 bp, Artificial Sequence
  Synthetic polynucleotide
atgctgtcgccgcgcgggg

SEQ ID NO: 78 - DNA, 19 bp, Artificial Sequence
  Synthetic polynucleotide
ccccgcgcggcgacagcau

SEQ ID NO: 79 - DNA, 19 bp, Artificial Sequence
  Synthetic polynucleotide
ctcaccctcaccggagcca

SEQ ID NO: 80 - DNA, 19 bp, Artificial Sequence
  Synthetic polynucleotide
uggcuccggugagggugag

SEQ ID NO: 81 - DNA, 19 bp, Artificial Sequence
  Synthetic polynucleotide
ccgcaaactttactcctta

SEQ ID NO: 82 - DNA, 19 bp, Artificial Sequence
  Synthetic polynucleotide
uaaggaguaaaguuugcgg

SEQ ID NO: 83 - DNA, 19 bp, Artificial Sequence
  Synthetic polynucleotide
ctcctaagattggcttcac

SEQ ID NO: 84 - DNA, 19 bp, Artificial Sequence
  Synthetic polynucleotide
gugaagccaaucuuaggag

SEQ ID NO: 85 - DNA, 19 bp, Artificial Sequence
  Synthetic polynucleotide
ccggagccactcctaagat

SEQ ID NO: 86 - protein, 718 aa, Homo sapiens
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 87 - DNA, 19 bp, Artificial Sequence
  Synthetic polynucleotide
ttctctaccctacgtctca

SEQ ID NO: 88 - DNA, 19 bp, Artificial Sequence
  Synthetic polynucleotide
ugagacguaggguagagaa

SEQ ID NO: 89 - DNA, 19 bp, Artificial Sequence
  Synthetic polynucleotide
tacgtctcattctccgcaa

SEQ ID NO: 90 - DNA, 19 bp, Artificial Sequence
  Synthetic polynucleotide
uugcggagaaugagacgua

SEQ ID NO: 91 - DNA, 19 bp, Artificial Sequence
  Synthetic polynucleotide
gctaggcctccagcccttc

SEQ ID NO: 92 - DNA, 19 bp, Artificial Sequence
  Synthetic polynucleotide
gaagggcuggaggccuagc

SEQ ID NO: 93 - DNA, 19 bp, Artificial Sequence
  Synthetic polynucleotide
acaggtggcgccgcaactt

SEQ ID NO: 94 - DNA, 19 bp, Artificial Sequence
  Synthetic polynucleotide
aaguugcggcgccaccugu

SEQ ID NO: 95 - DNA, 19 bp, Artificial Sequence
  Synthetic polynucleotide
agccggaggcgcgagagtc

SEQ ID NO: 96 - DNA, 19 bp, Artificial Sequence
  Synthetic polynucleotide
gacucucgcgccuccggcu

SEQ ID NO: 97 - protein, 720 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 98 - protein, 100 aa, Artificial Sequence
  Synthetic polypeptide
GGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSE

SEQ ID NO: 99 - protein, 2483 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFASGSGPKKKRKV

SEQ ID NO: 100 - protein, 261 aa, Artificial Sequence
  Synthetic polypeptide
SQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 101 - protein, 2750 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFASGSGPKKKRKV

SEQ ID NO: 102 - protein, 2216 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSPKKKRKVDPKKKRKVDPKKKRKV

SEQ ID NO: 103 - protein, 2116 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSPKKKRKVDPKKKRKVDPKKKRKVGSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 104 - protein, 285 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSDALDDFDLDMLGSDALDDFDLDMLGSDALDDFDLDMLGSDALDDFDLDMLASGSGPKKKRKV

SEQ ID NO: 105 - protein, 562 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSDALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLASGSGPKKKRKV

SEQ ID NO: 106 - protein, 758 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSDALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFASGSGPKKKRKV

SEQ ID NO: 107 - protein, 496 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLASGSGPKKKRKV

SEQ ID NO: 108 - protein, 425 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFASGSGPKKKRKV

SEQ ID NO: 109 - protein, 692 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLFASGSGPKKKRKV

SEQ ID NO: 110 - protein, 235 aa, Artificial Sequence
  Synthetic polypeptide
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYGGPSSGAPPPSGGSPAGSPTSTEEGTSESATPESGPGTSTEPSEGSAPGSPAGSPTSTEEGTSTEPSEGSAPGTSTEPSEGSGPKKKRKVAGSASGSGPKKKRKV

SEQ ID NO: 111 - protein, 2132 aa, Artificial Sequence
  Synthetic polypeptide
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSPKKKRKVDPKKKRKVDPKKKRKVGSGSETPGTSESATPESSLPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 112 - protein, 2132 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVSGSETPGTSESATPESMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSPKKKRKVDPKKKRKVDPKKKRKV

SEQ ID NO: 113 - protein, 2196 aa, Artificial Sequence
  Synthetic polypeptide
MALPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWVGGPSSGAPPPSGGSPAGSPTSTEEGTSESATPESGPGTSTEPSEGSAPGSPAGSPTSTEEGTSTEPSEGSAPGTSTEPSEMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGGSPKKKRKVDPKKKRKVDPKKKRKV

SEQ ID NO: 114 - DNA, 113 bp, Artificial Sequence
  Synthetic polynucleotide
gacgctcaaatttccgcagtgtttaagagctaagctggaaacagcatagcaagtttaaataaggctagtccgttatcaacttgaaaaagtggcaccgagtcggtgcttttttt

SEQ ID NO: 115 - DNA, 93 bp, Artificial Sequence
  Synthetic polynucleotide
gtttaagagctaagctggaaacagcatagcaagtttaaataaggctagtccgttatcaacttgaaaaagtggcaccgagtcggtgcttttttt

SEQ ID NO: 116 - DNA, 20 bp, Artificial Sequence
  Synthetic polynucleotide
gacgctcaaatttccgcagt

SEQ ID NO: 117 - DNA, 143 bp, Artificial Sequence
  Synthetic polynucleotide
gtttaagagctaagccaacatgaggatcacccatgtctgcagggcatagcaagtttaaataaggctagtccgttatcaacttggccaacatgaggatcacccatgtctgcagggccaagtggcaccgagtcggtgcttttttt

SEQ ID NO: 118 - DNA, 19 bp, Artificial Sequence
  Synthetic polynucleotide
taatacgactcactatagg

SEQ ID NO: 119 - DNA, 27 bp, Artificial Sequence
  Synthetic polynucleotide
agatcggaagagcacacgtctgaactc

SEQ ID NO: 120 - DNA, 19 bp, Artificial Sequence
  Synthetic polynucleotide
aucuuaggaguggcuccgg